(19) United States
(12) Patent Application Publication
(10) Pub. No.: US 2005/0148601 A1
(43) Pub. Date: Jul. 7, 2005

(54) Neurokinin-3 receptor modulators: diaryl imidazole derivatives

(76) Inventors: George D. Maynard, Clinton, CT (US); Stanislaw Rachwal, Irvine, CA (US); Wallace C. Pringle, Guilford, CT (US); Jun Yuan, Guilford, CT (US); Linghong Xie, Guilford, CT (US); Yuelian Xu, East Haven, CT (US); Bingsong Han, North Haven, CT (US)

Correspondence Address: CANTOR COLBURN, LLP, 55 GRIFFIN ROAD SOUTH, BLOOMFIELD, CT, 06002, US

(21) Appl. No.: 11/007,652

(22) Filed: Dec. 7, 2004

Related U.S. Application Data
(60) Provisional application No. 60/531,595, filed on Dec. 19, 2003.

(51) Int. Cl. A61K 31/538; A61K 31/506; A61K 31/498; A61K 31/4439; A61K 31/427; A61K 31/4178; C07D 43/02; C07D 413/02; C07D 417/02

(52) U.S. Cl. 514/256; 514/341; 514/365; 514/397; 514/400; 544/331; 546/272.7; 548/200; 548/311.1; 548/338.1; 544/353; 514/230.5; 514/249; 544/105

(57) ABSTRACT

The invention relates to compounds of general Formula I: 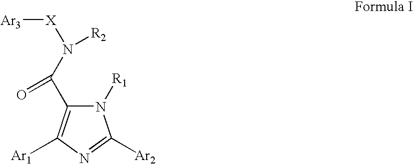 wherein the variables are as defined herein. Also provided are pharmaceutical compositions comprising such compounds, and methods for treating patients suffering from a disorder responsive to neurokinin-3 receptor modulation. NK-3 receptor modulators provided herein are also useful as probes for the localization of NK-3 receptors.

### PRIORITY INFORMATION

This application claims priority to U.S. Provisional Patent Application No. 60/531,595 filed Dec. 19, 2003, which is hereby incorporated by reference in its entirety.

### FIELD OF THE INVENTION

This invention relates generally to certain diaryl imidazole derivatives, pharmaceutical compositions comprising such compounds, and the use of such compounds in the treatment of certain diseases and disorders that are responsive to NK-3 receptor modulation. Compounds provided herein are further useful as probes for the localization of NK-3 receptors.

### BACKGROUND

The tachykinins are a family of structurally related peptides originally isolated based upon their smooth muscle contractile and sialogogic activity. These mammalian peptides include substance P (SP), neurokinin ai (neurokinin A; NKA) and neurokinin p (neurokinin B; NKB). Tachykinins are synthesized in the central nervous system (CNS) and in peripheral tissues, where they exert a variety of biological activities.

Three receptors for the tachykinin peptides have been characterized and are referred to as neurokinin-1 (NK-1), neurokinin-2 (NK-2) and neurokinin-3 (NK-3) receptors. The NK-1 receptor has a natural agonist potency profile of SP>NKA>NKB. The NK-2 receptor agonist potency profile is NKA>NKB>SP, and the NK-3 receptor agonist potency profile is NKB>NKA>SP. Each of the three receptors mediates a variety of tachykinin-stimulated biological effects, including 1) modulation of smooth muscle contractile activity, 2) transmission of (generally) excitatory neuronal signals in the CNS and periphery (e.g., pain signals), 3) modulation of immune and inflammatory responses, 4) induction of hypotensive effects via dilation of the peripheral vasculature, and 5) stimulation of endocrine and exocrine gland secretions. These receptors transduce intracellular signals via the activation of pertussis toxin-insensitive (G_{αq/11}) G proteins, resulting in the generation of the intracellular second messengers inositol 1,4,5-trisphosyphate and diacylglycerol. NK-1 receptors are expressed in a wide variety of peripheral tissues and in the CNS. NK-2 receptors are expressed primarily in the periphery, while NK-3 receptors are primarily (but not exclusively) expressed in the CNS, including the human brain.

NK-3 receptor antagonists show considerable potential for treating a variety of CNS and peripheral disorders. In the CNS, activation of NK-3 receptors has been shown to modulate dopamine and serotonin release, indicating therapeutic utility in the treatment of disorders such as anxiety, depression, schizophrenia and obesity. Further, studies in primate brain detect the presence of NK-3 mRNA in a variety of regions relevant to these disorders. With regard to obesity, it has also been shown that NK-3 receptors are located on melanin concentrating hormone-containing neurons in the rat lateral hypothalamus and zona incerta. In the periphery, administration of NKB into the airways is known to induce mucus secretion and bronchoconstriction, indicating therapeutic utility for NK-3 receptor antagonists in the treatment of patients suffering from airway diseases such as asthma and chronic obstructive pulmonary disease (COPD). Localization of NK-3 receptors in the gastrointestinal (GI) tract and the bladder indicates therapeutic utility for NK-3 receptor antagonists in the treatment of GI and bladder disorders including inflammatory bowel disease and urinary incontinence.

Both peptide and nonpeptide antagonists have been developed for each of the tachykinin receptors. Peptide antagonists for the tachykinin receptors have been characterized by low potency, partial agonism, poor metabolic stability and toxicity, but non-peptide antagonists have been found to display more drug-like properties. Unfortunately, non-peptide NK-3 receptor antagonists have suffered from other disadvantages including species selectivity, which limits the evaluation of these compounds in appropriate disease models. There is thus a need for new non-peptide NK-3 receptor antagonists for use as therapeutic agents, and as tools to investigate the anatomical and ultrastructural distribution of NK-3 receptors, as well as the physiologic and pathophysiologic consequences of NK-3 receptor activation. The present invention fulfills this need, and provides further related advantages.

### SUMMARY OF THE INVENTION

The present invention provides diaryl imidazole derivatives that are characterized by the formula: 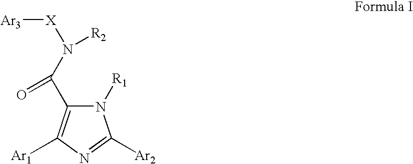
or are a pharmaceutically acceptable salt of such a compound. Within Formula I,
X is N(R₃ₐ) or C(R₃ₐ)(R_{3b});
Ar₁ is a 6- to 10-membered aryl, a 5- to 10-membered heteroaryl, or a group of the formula 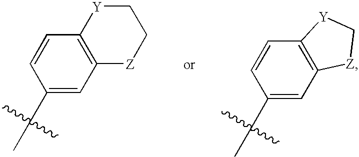
   wherein Y and Z are independently CH₂, NH, O or S;
each of which is substituted with from 0 to 4 substituents independently chosen from halogen, cyano, nitro, oxo and groups of the formula L-M;
Ar₂ is a 6- to 1 o-membered aryl or 5- to 10-membered heteroaryl, each of which is substituted with from 0 to 4 substituents independently chosen from halogen, cyano, nitro, oxo and groups of the formula L-M;
Ar₃ is a 6- to 10-membered aryl or 6- to I O-membered heteroaryl, each of which is substituted with from 0 to 4 substituents independently chosen from:
   (i) halogen, cyano, nitro and oxo;
   (ii) groups of the formula L-M; and
   (iii) groups that are taken together with R₃ₐ or R_{3b} to form a fused, partially saturated 5- to 8-membered carbocycle or heterocycle;
R₁ is C₁-C₆alkyl substituted with from 0 to 6 substituents independently chosen from halogen, cyano, nitro, oxo and groups of the formula L-M; or R₁ is taken together with R₂ to form a fused, optionally substituted 5- to 7-membered heterocycle;
R₂ is:
   (i) hydrogen, C₁-C₆alkyl or C₁-C₆alkenyl, each of which alkyl and alkenyl is substituted with from 0 to 6 substituents independently chosen from R_{b};
   (ii) taken together with R₃ₐ or R_{3b} to form a 4- to 8-membered heterocycloalkyl substituted with from 0 to 4 substituents independently chosen from halogen, cyano, nitro, oxo and groups of the formula L-M; or
   (iii) taken together with R₁ to form a fused, optionally substituted 5- to 7-membered heterocycle;
R₃ₐ and R_{3b} are:
   (i) independently
      (a) hydrogen, C₁-C₈alkyl, C₁-C8alkenyl, C₃-C₈cycloalkyl or phenyl, wherein each alkyl, alkenyl, cycloalkyl and phenyl is substituted with from 0 to 6 substituents independently chosen from halogen, cyano, nitro, oxo and groups of the formula L-M;
      (b) taken together with R₂ to form an optionally substituted 4- to 8-membered heterocycloalkyl; or
      (c) taken together with a substituent of Ar₃ to form a fused, partially saturated, 5- to 8-membered carbocycle or heterocycle, each of which is substituted with from 0 to 4 substituents independently chosen from halogen, cyano, nitro, oxo and groups of the formula L-M; or
   (ii) taken together to form a spiro 3- to 8-membered carbocycle or heterocycle;
      preferably at least one of R₃ₐ and R_{3b} is not hydrogen;
L is independently selected at each occurrence from a bond, 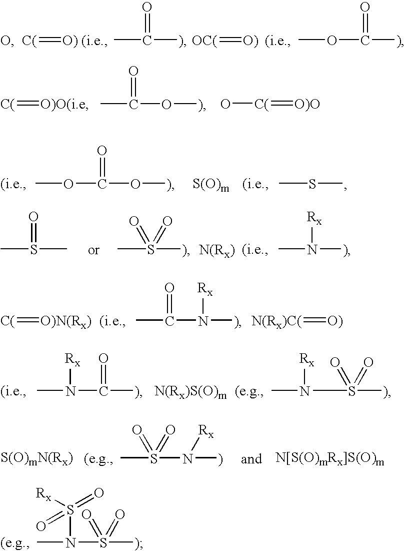
   wherein m is independently selected at each occurrence from 0, 1 and 2; and Rₓ is independently selected at each occurrence from hydrogen and C₁-C₈alkyl;
M is independently selected at each occurrence from: (i) hydrogen; and (ii) C₁-C₆alkyl, C₂-C₆alkenyl, C₂-C₆alkynyl, phenylCo-C₆alkyl and (3- to 10-:membered heterocycle)C₀-C₆alkyl, each of which is substituted with from 0 to 6 substituents independently chosen from R_{b}; and
R_{b} is independently chosen at each occurrence from:
   (i) hydroxy, halogen, amino, aminocarbonyl, cyano, nitro, oxo and —COOH; and
   (ii) C₁-C₆alkyl, C₁-C₆alkenyl, C₁-C₆alkynyl, haloC₁-C₆alkyl, hydroxyC₁-C₆alkyl, cyanoC₁-C₈alkyl, C₁-C₆alkoxy, haloC₁-C₈alkoxy, C₁-C₆alkanoyl, C₂-C₆alkoxycarbonyl, C₂-C₆alkanoyloxy, C₁-C₆alkylthio, C₂-C₆alkyl ether, 4- to 8-membered heterocycloalkyl, and mono- and di-(C₁-C₆alkyl)aminoC₀-C₆alkyl.

Within certain aspects, methods are provided for using one or more compounds provided herein to treat a patient suffering from a condition that is responsive to NK-3 receptor modulation. Such conditions include central nervous system and peripheral diseases and disorders including, but not limited to, anxiety, depression, psychosis, obesity, chronic pulmonary obstructive disorder, gastrointestinal conditions such as irritable bowel syndrome or colitis, pain and cognitive disorders (e.g., cognition impairment, mild cognitive impairment (MCI), age-related cognitive decline (ARCD), traumatic brain injury, Down's Syndrome, neurodegenerative diseases such as Alzheimer's disease and Parkinson's disease, stroke, AIDS associated dementia, and dementia associated with depression, anxiety and psychosis (including schizophrenia and hallucinatory disorders). Treatment of humans, domesticated companion animals (pets) or livestock animals suffering from such conditions with a therapeutically effective amount of at least one compound provided herein is contemplated by the present invention.

In further aspects, methods are provided herein for using one or more compounds provided herein to treat a patient suffering from a condition that is responsive to CB1 modulation. Such conditions include appetite disorders, obesity, addictive disorders, asthma, liver cirrhosis, sepsis, irritable bowel disease, Crohn's disease, depression, memory disorders, cognitive disorders and movement disorders. Treatment of humans, domesticated companion animals (pets) or livestock animals suffering from such conditions with a therapeutically effective amount of a compound provided herein is contemplated by the present invention. In a related aspect, methods are provided for suppressing appetite in a patient, comprising administering to the patient an appetite reducing amount of at least one compound provided herein.

Within further aspects, the present invention provides pharmaceutical compositions comprising one or more compounds provided herein. Packaged pharmaceutical compositions comprising a pharmaceutical composition and instructions for use of the composition to treat a condition that is responsive to CB1 or NK-3 receptor modulation are also provided.

In a separate aspect, the present invention provides methods for potentiating the action of other CNS active compounds. Such methods comprise administering to a patient a therapeutically effective amount of a compound provided herein in combination with a second CNS active compound.

The present invention further provides methods for using the compounds provided herein as positive controls in assays for NK-3 receptor activity and for using appropriately labeled compounds as probes for the localization of NK-3 receptors (e.g., in tissue sections).

### DETAILED DESCRIPTION OF THE INVENTION

As noted above, the present invention provides diaryl imidazole derivatives of Formula I, including the pharmaceutically acceptable salts of such compounds. In certain aspects, such compounds are NK-3 receptor modulators and may be used in vivo or in vitro to modulate NK-3 receptor activity in a variety of contexts.

### TERMINOLOGY

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Certain compounds are described herein using a general formula that includes variables (e.g., Ar₁, R₁). Unless otherwise specified, each variable within such a formula is defined independently of any other variable, and any variable that occurs more than one time in a formula is defined independently at each occurrence.

A “pharmaceutically acceptable salt” is an acid or base salt form of a compound, which salt form is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, EtOAc, EtOH, isopropanol or acetonitrile, is preferred.

It will be apparent that each compound of Formula I may, but need not, be formulated as a hydrate, solvate or non-covalent complex. In addition, the various crystal forms and polymorphs are within the scope of the present invention. Also provided herein are prodrugs of the compounds of Formula I. A “prodrug” is a compound that may not fully satisfy the structural requirements of the compounds provided herein, but is modified in vivo, following administration to a patient, to produce a compound of Formula I, or other formula provided herein. For example, a prodrug may be an acylated derivative of a compound as provided herein. Prodrugs include compounds wherein hydroxy, amine or sulffiydryl groups are bonded to any group that, when administered to a mammalian subject, cleaves to form a free hydroxy, amino or sulfhydryl group, respectively. Examples of prodrugs include, but are not limited to, acetate, formate and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

A “substituent,” as used herein, refers to a molecular moiety that is covalently bonded to an atom within a molecule of interest. For example, a “ring substituent” may be a moiety such as a halogen, alkyl group, haloalkyl group or other substituent discussed herein that is covalently bonded to an atom (preferably a carbon or nitrogen atom) that is a ring member. The term “substitution” refers to replacing a hydrogen atom in a molecular structure with a substituent as described above, such that the valence on the designated atom is not exceeded, and such that a chemically stable compound (i.e., a compound that can be isolated, characterized, and tested for biological activity) results from the substitution. When a substituent is oxo (i.e., ═O), then 2 hydrogens on the atom are replaced. When aromatic moieties are substituted with an oxo group, the aromatic ring is replaced by the corresponding partially unsaturated ring. For example, a pyridyl group substituted with oxo is a pyridone.

The phrase “optionally substituted” indicates that a group may either be unsubstituted or substituted at one or more of any of the available positions, typically 1, 2, 3, 4 or 5 positions, by one or more suitable substituents such as those disclosed herein. Optional substitution is also indicated by the phrase “substituted with from 0 to X substituents,” in which X is the maximum number of substituents.

A dash (“—”) that is not between two letters or symbols is used to indicate a point of attachment for a substituent. For example, —C(═O)NH₂ is attached through the carbon atom.

As used herein, the term “alkyl” refers to a straight chain, branched chain or cyclic saturated aliphatic hydrocarbon. An alkyl group may be bonded to an atom within a molecule of interest via any chemically suitable portion. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, cyclopropyl, cyclopropylmethyl, cyclopentyl, cyclopentylmethyl, cyclohexyl, cycloheptyl and norbomyl. “C₀-C₄alkyl” refers to a bond (C₀) or an alkyl group having 1, 2, 3 or 4 carbon atoms; “C₀-C₆alkyl” refers to a bond or a C₁-C₆alkyl group; “C₀-C₈alkyl” refers to a bond or a C₁-C₈alkyl group. In certain embodiments, alkyl groups are straight or branched chain. In some instances herein, a substituent of an alkyl group is specifically indicated. For example, “cyanoC₁-C₄alkyl” refers to a C₁-C₄alkyl group that has a CN substituent. One representative branched cyanoalkyl group is —C(CH₃)₂CN.

Similarly, “alkenyl” refers to straight or branched chain alkene groups or cycloalkene groups, in which at least one unsaturated carbon-carbon double bond is present. Alkenyl groups include C₂-C₈alkenyl, C₂-C₆alkenyl and C₂-C₄alkenyl groups, which have from 2 to 8, 2 to 6 or 2 to 4 carbon atoms, respectively, such as ethenyl, allyl or isopropenyl. In certain embodiments, alkenyl groups are straight or branched chain. “Alkynyl” refers to straight or branched chain alkyne groups, which have one or more unsaturated carbon-carbon bonds, at least one of which is a triple bond. Alkynyl groups include C₂-C₈alkynyl, C₂-C₆alkynyl and C₂-C₄alkynyl groups, which have from 2 to 8, 2 to 6 or 2 to 4 carbon atoms, respectively. In certain embodiments, preferred alkenyl and alkynyl groups are straight or branched chain.

The term “alkylene” refers to a divalent alkyl group.

By “alkoxy,” as used herein, is meant an alkyl group as described above attached via an oxygen bridge. Alkoxy groups include C₁-C₈alkoxy, C₁-C₆alkoxy and C₁-C₄alkoxy groups, which have from 1 to 8, 1 to 6 or 1 to 4 carbon atoms, respectively. Alkoxy groups include, for example, methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy. Similarly, “alkylthio” refers to an alkyl group as described above attached via a sulfur bridge.

The term “alkanoyl” refers to an acyl group in a linear, branched or cyclic arrangement (e.g., —(C═O)-alkyl). Alkanoyl groups include C₂-C₈alkanoyl, C₂-C₆alkanoyl, and C₂-C₄alkanoyl groups, which have from 2 to 8, 2 to 6 or 2 to 4 carbon atoms, respectively. “Cialkanoyl” refers to —C═O)—H, which (along with C₂-C₈alkanoyl) is encompassed by the term “C₁-C₈alkanoyl.”

An “alkanone” is a ketone group in which carbon atoms are in a linear, branched or cyclic alkyl arrangement. “C₃-C₈alkanone,” “C₃-C₆alkanone” and “C₃-C₄alkanone” refer to an alkanone having from 3 to 8, 6, or 4 carbon atoms, respectively. By way of example, a C₃ alkanoine group has the structure —CH₂—(C═O)—CH₃.

Similarly, “alkylether” refers to a linear or branched ether substituent linked via a carbon-carbon bond. Alkyl ether groups include C₂-C₈alkylether, C₂-C₆alkylether, and C₂-C₄alkylether groups, which have 2 to 8, 6 or 4 carbon atoms, respectively. By way of example, a C₂ alkyl ether group has the structure —CH₂—O—CH₃. A representative branched alkyl ether substituent is —C(CH₃)₂CH₂—O—CH₃.

The term “alkoxycarbonyl” refers to an alkoxy group linked via a carbonyl (i.e., a group having the general structure —C(═O—O-alkyl). Alkoxycarbonyl groups include C₂-C₈, C₂-C₆, and C₂-C₄alkoxycarbonyl groups, which have from 2 to 8, 6, or 4 carbon atoms, respectively. “Cialkoxycarbonyl” refers to —C(═O)—OH, which is encompassed by the term “C₁-C₈alkoxycarbonyl.”

“Alkanoyloxy,” as used herein, refers to an alkanoyl group linked via an oxygen bridge (i.e., a group having the general structure —O—C(═O)alkyl). Alkanoyloxy groups include C₂-C₈, C₂-C₆, and C₂-C₄alkanoyloxy groups, which have from 2 to 8, 6, or 4 carbon atoms, respectively.

“Alkylamino” refers to a secondary or tertiary amine having the general structure —NH-alkyl or -N(alkyl)(alkyl), wherein each alkyl may be the same or different. Such groups include, for example, mono- and di-(C₁-C₈alkyl)amino groups, in which each alkyl may be the same or different and may contain from 1 to 8 carbon atoms, as well as mono- and di-(C₁-C₆alkyl)amino groups and mono- and di-(C₁-C₄alkyl)amino groups.

“Alkylaminoalkyl” refers to an alkylamino group linked via an alkyl group (i.e., a group having the general structure -alkyl-NH-alkyl or -alkyl-N(alkyl)(alkyl)) in which each alkyl is selected independently. Such groups include, for example, mono- and di-(C₁-C₆alkyl)aminoC₁-C₆alkyl in which each alkyl may be the same or different. “Mono- or di-(C₁-C₆alkyl)aminoCO-C₆alkyl” refers to a mono- or di-(C₁-C₆alkyl)amino group linked via a direct bond or a C₁-C₆alkyl group. The following are representative alkylaminoalkyl groups:

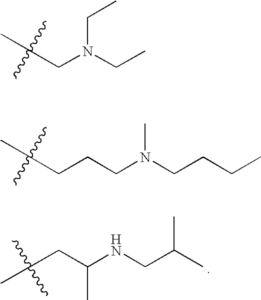

The term “aminocarbonyl” refers to an amide group (i.e., —(C═O)NH₂).

The term “hydroxyalkyl” refers to an alkyl group substituted with at least one hydoxyl substituent. The term “cyanoalkyl” refers to an alkyl group substituted with at least one cyano substituent.

The term “halogen” refers to fluorine, chlorine, bromine and iodine.

A “haloalkyl” is a branched, straight-chain or cyclic alkyl group, substituted with 1 or more halogen atoms (e.g., “haloC₁-C₈alkyl” groups have from 1 to 8 carbon atoms; “haloC₁-C₆alkyl” groups have from 1 to 6 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; mono-, di-, tri-, tetra- or penta-chloroethyl; and 1,2,2,2-tetrafluoro-1-trifluoromethyl-ethyl. Typical haloalkyl groups are trifluoromethyl and difluoromethyl. The term “haloalkoxy” refers to a haloalkyl group as defined above attached via an oxygen bridge. “HaloC₁-C₈alkoxy” groups have 1 to 8 carbon atoms.

A “heteroatom,” as used herein, is oxygen, sulfur or nitrogen.

A “carbocycle” or “carbocyclic group” comprises at least one ring formed entirely by carbon-carbon bonds (referred to herein as a carbocyclic ring), and does not contain a heterocyclic ring. Unless otherwise specified, each carbocyclic ring within a carbocycle may be saturated, partially saturated or aromatic. A carbocycle generally has from 1 to 3 fused, pendant or spiro rings; carbocycles within certain embodiments have one ring or two fused rings. Typically, each ring contains from 3 to 8 ring members (i.e., C₃-C₈); C₅-C₇ rings are recited in certain embodiments. Carbocycles comprising fused, pendant or spiro rings typically contain from 9 to 14 ring members. Certain representative carbocycles are cycloalkyl (i.e., groups that comprise only saturated and/or partially saturated rings, such as cyclopropyl, cyclobutyl, cyclopentyl, cyclohexyl, cycloheptyl, cyclooctyl, adamantyl, decahydro-naphthalenyl, octahydro-indenyl, and partially saturated variants of any of the foregoing, such as cyclohexenyl). Other carbocycles are aryl (i.e., contain at least one aromatic carbocyclic ring). Such carbocycles include, :for example, phenyl, naphthyl, fluorenyl, indanyl and 1,2,3,4-tetrahydro-naphthyl. PhenylC0-C2alkyl refers to a phenyl group linked via a single covalent bond or a C1-C2alkylene group.

A “heterocycle” or “heterocyclic group” has from 1 to 3 fused, pendant or spiro rings, at least one of which is a heterocyclic ring (i.e., one or more ring atoms is a heteroatom, with the remaining ring atoms being carbon). Typically, a heterocyclic ring comprises 1, 2, 3, or 4 heteroatoms; within certain embodiments each heterocyclic ring has 1 or 2 heteroatoms per ring. Each heterocyclic ring generally contains from 3 to 8 ring members (rings having from 4 or 5 to 7 ring members are recited in certain embodiments) and heterocycles comprising fused, pendant or spiro rings typically contain from 9 to 14 ring members. Certain heterocycles comprise a sulfur atom as a ring member; in certain embodiments, the sulfur atom is oxidized to SO or SO2. Heterocycles may be optionally substituted with a variety of substituents, as indicated. Unless otherwise specified, a heterocycle may be a heterocycloalkyl group (i.e., each ring is saturated or partially saturated) or a heteroaryl group (i.e., at least one ring within the group is aromatic). A heterocyclic group may generally be linked via any ring or substituent atom, provided that a stable compound results. N-linked heterocyclic groups are linked via a component nitrogen atom.

Heterocyclic groups include, for example, azepanyl, azocinyl, benzimidazolyl, benzimidazolinyl, benzisothiazolyl, benzisoxazolyl, benzofuranyl, benzothiofuranyl, benzoxazolyl, benzothiazolyl, benztetrazolyl, chromanyl, chromenyl, cinnolinyl, decahydroquinolinyl, dihydrofuro[2,3-b]tetrahydrofuranyl, dihydroisoquinolinyl, dihydrotetrahydrofuranyl, 1,4-dioxa-8-aza-spiro[4.5]decyl, dithiazinyl, furanyl, furazanyl, imidazolinyl, imidazolidinyl, imidazolyl, indazolyl, indolenyl, incdolinyl, indolizinyl, indolyl, isobenzofuranyl, isochromanyl, isoindazolyl, isoindolinyl, isoindolyl, isothiazolyl, isoxazolyl, isoquinolinyl, morpholinyl, naphthyridinyl, octahydroisoquinolinyl, oxadiazolyl, oxazolidinyl, oxazolyl, phthalazinyl, piperazinyl, piperidinyl, piperidinyl, piperidonyl, pteridinyl, purinyl, pyranyl, pyrazinyl, pyrazolidinyl, pyrazolinyl, pyrazolyl, pyri dazinyl, pyridoimidazolyl, pyridooxazolyl, pyridothiazolyl, pyridyl, pyrimidyl, pyrrolidinyl, pyrrolidonyl, pyrrolinyl, pyrrolyl, quinazolinyl, quinolinyl, quinoxalinyl, quinuclidinyl, tetrahydroisoquinolinyl, tetrahydroquinolinyl, tetrazolyl, thiadiazinyl, thiadiazolyl, thiazolyl, thienothiazolyl, thienooxazolyl, thienoimidazolyl, thienyl, thiophenyl, thiomorpholinyl and variants thereof in which the sulfur atom is oxidized, triazinyl, and any of the foregoing that are substituted with from 1 to 4 substituents as described above.

Certain heterocyclic groups are 3- to 8-membered, 5- to 10-membered, 6- to 10-membered, 4- to 8-membered, 5- to 8-membered, or 5- to 7-membered groups that contain 1 heterocyclic ring or 2 fused or spiro rings, optionally substituted. 5- to 8-membered heterocycloalkyl groups include, for example, piperidinyl, piperazinyl, pyrrolidinyl, azepanyl, morpholino, thiomorpholino and 1,1-dioxo-thiomorpholin-4-yl. Such groups may be substituted as indicated. Representative aromatic heterocycles are azocinyl, pyridyl, pyrimidyl, imidazolyl, tetrazolyl and 3,4-dihydro-1H-isoquinolin-2-yl.

The term “NK-3 receptor” is used herein to refer to human neurokinin-3 receptor (see Huang et al. (1992) Biochem. Biophys. Res. Commin. 184: 966-72; Regoli et al. (1994) Pharmacol. Rev. 46:551-99), as well as homologues thereof found in other species.

A “NK-3 receptor modulator,” also referred to herein as a “modulator,” is a compound that modulates NK-3 receptor activation and/or NK-3-mediated signal transduction. NK-3 receptor modulators specifically provided herein are compounds of Formula I and pharmaceutically acceptable salts of such compounds. A NK-3 receptor modulator may be a NK-3 receptor agonist or antagonist. A modulator binds with “high affinity” if the Ki at NK-3 receptor is less than 1 micromolar, preferably less than 500 nanomolar, 100 nanomolar or 10 nanomolar. A representative assay for determining Ki at NK-3 receptor is provided in Example 4, herein.

A modulator is considered an “antagonist” if it detectably inhibits NK-3 ligand (e.g., neurokinin) binding to NK-3 receptor and/or NK-3 receptor-mediated signal transduction (using, for example, the representative assay provided in Example 4); in general, such an antagonist inhibits NK-3 receptor activation with a IC50 value of less than 1 micromolar, in some embodiments less than 500 nanomolar, and in certain embodiments less than 100 nanomolar or 10 nanomolar within the assay provided in Example 5. NK-3 receptor antagonists include neutral antagonists and inverse agonists.

An “inverse agonist” of NK-3 receptor is a compound that reduces the activity of NK-3 receptor below its basal activity level in the absence of added NK-3. Inverse agonists of NK-3 receptor may also inhibit the activity of neurokinin at NK-3 receptor, and/or may also inhibit binding of neurokinin to NK-3 receptor. The ability of a compound to inhibit the binding of neurokinin to NK-3 receptor may be measured by a binding assay, such as the binding assay given in Example 4. The basal activity of NK-3 receptor, as well as the reduction in NK-3 receptor activity due to the presence of NK-3 inverse agonist, may be determined from a calcium mobilization assay, such as the assay of Example 5.

A “neutral antagonist” of NK-3 receptor is a compound that inhibits the activity of NK-3 at NK-3 receptor, but does not significantly change the basal activity of the receptor (i.e., within a calcium mobilization assay as described in Example 5 performed in the absence of NK-3, receptor activity is reduced by no more than 10%, more preferably by no more than 5%, and even in some embodiments by no more than 2%; preferably, there is no detectable reduction in activity). Neutral antagonists of NK-3 receptor may inhibit the binding of NK-3 to the receptor.

As used herein a “NK-3 receptor agonist” is a compound that elevates the activity of the receptor above the basal activity level of the receptor (i.e., enhances NK-3 receptor activation and/or NK-3 mediated signal transduction). NK-3 receptor agonist activity may be identified using the representative assay provided in Example 5. hi general, such an agonist has an EC₅₀ value of less than 1 micromolar, preferably less than 500 nanomolar, and more preferably less than 100 nanomolar within the assay provided in Example 5.

A “therapeutically effective amount” (or dose) is an amount that, upon administration to a patient, results in a discernible patient benefit. Such an amount or dose generally results in a concentration of compound in a body fluid (e.g., blood, plasma, serum, CSF, synovial fluid, lymph, cellular interstitial fluid, tears or urine) that is sufficient to inhibit the binding of NK-3 receptor ligand to NK-3 in vitro, as determined using the assay described in Example 4. It will be apparent that the therapeutically effective amount for a compound will depend upon the indication for which the compound is administered, as well as any co-administration of other therapeutic agent(s). The patient benefit may be detected using any appropriate criteria, including alleviation of one or more symptoms. It will be apparent that the patient benefit may be apparent after administration of a single dose, or may become apparent following repeated administration of the therapeutically effective dose according to a prescribed regimen, depending upon the indication for which the compound is administered.

A “patient” is any individual treated with a NK-3 receptor modulator as provided herein. Patients include humans, as well as other animals such as companion animals (e.g., dogs and cats) and livestock. Patients may be experiencing one or more symptoms of a condition responsive NK-3 receptor modulation (e.g., anxiety, depression, psychosis, obesity, chronic pulmonary obstructive disorder, gastrointestinal conditions such as irritable bowel syndrome or colitis, pain and cognitive disorders as described above), or may be free of such symptom(s) (i.e., treatment may be prophylactic).

### DIARYL IMIDAZOLE DERIVATIVES

As noted above, the present invention provides compounds of Formula I, with the variables as described above, as well as pharmaceutically acceptable salts of such compounds. 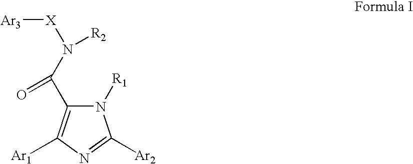
Certain preferred compounds are NK-3 receptor antagonists and have no detectable agonist activity in the assay described in Example 5. Preferred compounds further bind with high affinity to NK-3 receptor.

Within certain compounds of Formula I, X is N(R₃ₐ) and R₃ₐ is hydrogen, methyl, ethyl or propyl. Other compounds of Formula I further satisfy Formula Ia:

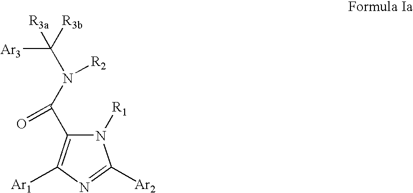

R₃ₐ of Formula Ia is, in certain embodiments, hydrogen. In certain such compounds, R_{3b} is C₁-C₆alkyl, C₁-C₆alkenyl, C₁-C₆alkynyl or phenylC₀-C₂alkyl (i.e., phenyl, benzyl or phenethyl), each of which is substituted with from 0 to 3 substituents independently chosen from halogen, hydroxy and cyano. Representative such R_{3b} groups include C₁-C₄alkyl groups such as methyl, ethyl, propyl, isopropyl and n-butyl. In other compounds of Formula Ia, R₃ₐ is hydrogen and R_{3b} is taken together with a substituent of Ar₃ to form an optionally substituted, fused 6- or 7-membered carbocycle or heterocycle. For example, in certain such compounds, the group represented by 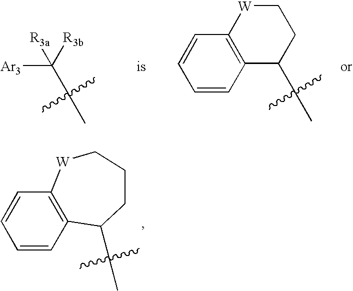
each of which is substituted with from 0 to 2 substituents independently chosen from methyl, ethyl and oxo, wherein W is CH₂, NH, O or S.

Certain compounds of Formula I further satisfy Formula II, in which R₃ is C₁-C₆alkyl that is substituted with from 0 to 3 substituents independently chosen from halogen, hydroxy and cyano, and the remaining variables are as described above.

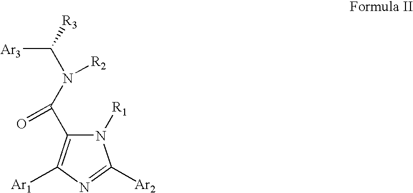

Within Formulas I, Ia and II, R₁ and R₂ are preferably independently hydrogen or C₁-C₄alkyl. In certain embodiments, R₁ is methyl or ethyl; in further embodiments, R₂ is hydrogen.

Ar₁, in certain compounds of Formulas I, Ia and II, is Ar₁ is phenyl, thiazolyl, thiophenyl, 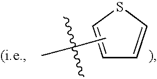
furenyl, pyridyl, pyrimidyl, naphthyl 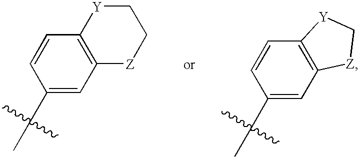
wherein Y and Z are independently CH₂, NH, O or S; each of which is substituted with from 0 to 4 substituents independently chosen from: (i) hydroxy, halogen, cyano, amino, C₁-C₆alkyl, C₁-C₆alkenyl, C₁-C₆alkynyl, haloC₁-C₆alkyl, hydroxyC₁-C₆alkyl, cyanoC₁-C₆alkyl, C₁-C₆alkoxy, C₁-C₆alkylthio, C₂-C₆alkyl ether, haloC₁-C₆alkoxy, and mono- or di-(C₁-C₆alkyl)aminoC₀-C₆alkyl; and (ii) phenyl, benzyl, or phenoxy, each of which is optionally substituted with halogen, C₁-C₄alkyl, or C₁-C₄alkoxy. It will be apparent that substitution may occur at any stable position, including at CH₂ and/or NH moieties located at Y and Z. Representative Ar₁ groups include, for example, phenyl, thiazolyl, and pyridyl, each of which is substituted with from 0 to 3 substituents independently chosen from halogen, C₁-C₄alkyl, haloC₁-C₄alkyl, C₁-C₄alkoxy, C₁-C₄alkylthio, and haloC₁-C₄alkoxy.

Ar₂, in certain compounds of Formulas I, Ia, and II, is phenyl, thiazolyl, pyridyl, or pyrimidyl, each of which is substituted with from 0 to 4 substituents independently chosen from hydroxy, halogen, cyano, amino, C₁-C₆alkyl, haloC₁-C₆alkyl, C₁-C₆alkoxy, and haloC₁-C₆alkoxy. Representative such groups include phenyl, thiazolyl, and pyridyl, each of which is unsubstituted or substituted with a halogen.

Ar₃, in certain compounds of Formulas I, Ia, and II, is phenyl, naphthyl or pyridyl, each of which is substituted with from 0 to 4 substituents independently chosen from hydroxy, halogen, cyano, amino, C₁-C₆alkyl, haloC₁-C₆alkyl, hydroxyC₁-C₆alkyl, cyanoC₁-C₆alkyl, C₁-C₆alkoxy, and haloC₁-C₆alkoxy. Representative such groups include phenyl that is unsubstituted or substituted with halogen, C₁-C₄alkyl or C₁-C₄alkoxy.

In further compounds of Formulas I, Ia, and II, Ar₁, Ar₂ and Ar₃ are independently chosen from phenyl and 6-membered heteroaryl, each of which is substituted with from 0 to 4 substituents independently chosen from hydroxy, halogen, cyano, amino, C₁-C₆alkyl, C₁-C₆alkenyl, C₁-C₆alkynyl, haloC₁-C₆alkyl, hydroxyC₁-C₆alkyl, cyanoC₁-C₈alkyl, C₁-C₆alkoxy, haloC₁-C₈alkoxy, C₁-C₆alkylthio, C₂-C₆alkylether, and mono- and di-(C₁-C₆alkyl)aminoC₀-C₆alkyl.

Certain compounds of Formula I further satisfy one or more of Formulas III-VIII:

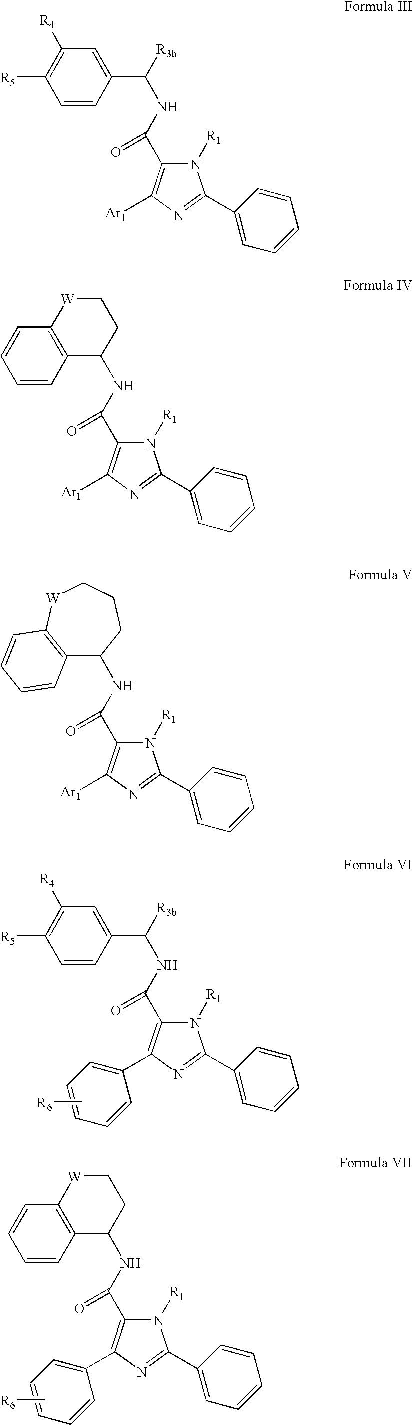

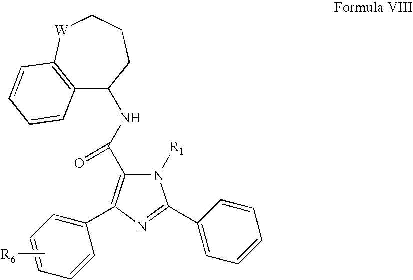

In Formulas III and VI, R₄ and R₅ are independently chosen from hydrogen, halogen, C₁-C₄alkyl, and C₁-C₄alkoxy, and R_{3b} may be C₁-C₄alkyl. R₆, in Formulas VI-VIII represents from 0 to 3 substituents that in some embodiments are independently chosen from halogen, C₁-C₄alkyl, haloC₁-C₄alkyl, C₁-C₄alkoxy, C₁-C₄alkylthio, and haloC₁-C₄alkoxy.

Representative compounds of Formula I, and subformulas thereof, include, but are not limited to, those specifically described in Examples 1-3. It will be apparent that the specific compounds recited therein are representative only, and are not intended to limit the scope of the present invention. Further, as noted above, all compounds of the present invention may be present as a pharmaceutically acceptable salt.

Certain compounds provided herein detectably alter (modulate) NK-3 receptor activity, as determined using an in vitro NK-3 receptor ligand binding assay (Example 4) and/or a functional assay such as a calcium mobilization assay (Example 5; also referred to herein as a “signal transduction assay”). Briefly, to assess binding to NK-3 receptor, a competition assay may be performed in which a NK-3 receptor preparation is incubated with labeled (e.g., ¹²⁵I or ³H) compound that binds to NK-3 receptor (e.g., a NK-3 receptor agonist such as neurokinin B or a variant thereof) and unlabeled test compound. Within the assays provided herein, the NK-3 receptor used is preferably mammalian, more preferably human or rat NK-3 receptor. The receptor may be recombinantly expressed or naturally expressed. The NK-3 receptor preparation may be, for example, a membrane preparation from HEK293 or CHO cells that recombinantly express human NK-3 receptor. Incubation with a compound that detectably modulates ligand binding to NK-3 receptor results in a decrease or increase in the amount of label bound to the NK-3 receptor preparation, relative to the amount of label bound in the absence of the compound. This decrease or increase may be used to determine the Kᵢ at NK-3 receptor as described herein. In general, compounds that decrease the amount of label bound to the NK-3 receptor preparation within such an assay are preferred.

As noted above, compounds that are NK-3 receptor antagonists are preferred in certain embodiments. IC₅₀ values for such compounds may be determined using a standard in vitro NK-3 receptor-mediated calcium mobilization assay, as provided in Example 5. Briefly, cells expressing NK-3 receptor are contacted with a compound of interest and with an indicator of intracellular calcium concentration (e.g., a membrane permeable calcium sensitivity dye such as Fluo-3 or Fura-2 (both of which are available, for example, from Molecular Probes, Eugene, Oreg.), each of which produce a fluorescent signal when bound to Ca⁺⁺). Such contact is preferably carried out by one or more incubations of the cells in buffer or culture medium comprising either or both of the compound and the indicator in solution. Contact is maintained for an amount of time sufficient to allow the dye to enter the cells (e.g., 1-2 hours). Cells are washed or filtered to remove excess dye and are then contacted with a NK-3 receptor agonist (e.g., neurokinin B or an analog thereof), typically at a concentration equal to the EC₅₀ concentration, and a fluorescence response is measured. When agonist-contacted cells are contacted with a compound that is a NK-3 receptor antagonist, the fluorescence response is generally reduced by at least 20%, preferably at least 50% and more preferably at least 80%, as compared to cells that are contacted with the agonist in the absence of test compound. The IC₅₀ for NK-3 receptor antagonists provided herein is preferably less than 1 micromolar, less than 500 riM, less than 100 riM or less than 10 nM.

In other embodiments, compounds that are NK-3 receptor agonists are preferred. When cells are contacted with 1 micromolar of a compound that is a NK-3 receptor agonist, the fluorescence response is generally increased by an amount that is at least 30% of the increase observed when cells are contacted with neurokinin B. The EC₅₀ for NK-3 receptor agonists provided herein is preferably less than 1 micromolar, less than 100 nM or less than 10 nM.

Preferred compounds provided herein are non-sedating. In other words, a dose of compound that is twice the minimum dose sufficient to provide a therapeutic effect, causes only transient (i.e., lasting for no more than ½ the time that the therapeutic effect lasts) or preferably no statistically significant sedation in an animal model assay of sedation (using the method described by Fitzgerald et al. (1988) Toxicology 49(2-3):433-9). Preferably, a dose that is five times the minimum dose sufficient to provide therapeutic effect does not produce statistically significant sedation. More preferably, compounds provided herein do not produce sedation at intravenous doses of 10 mg/kg or 25 mg/kg, or at oral doses of 30 mg/kg, 50 mg/kg, or 140 mg/kg.

In certain embodiments, preferred compounds provided herein have favorable pharmacological properties, including oral bioavailability (such that a sub-lethal or preferably a pharmaceutically acceptable oral dose, preferably less than 2 grams, more preferably less than or equal to one gram or 200 mg, can provide a detectable in vivo effect), low toxicity (a preferred compound is nontoxic when a therapeutically effective amount is administered to a subject), minimal side effects (a preferred compound produces side effects comparable to placebo when a therapeutically effective amount of the compound is administered to a subject), low serum protein binding, and a suitable in vitro and in vivo half-life (a preferred compound exhibits an in vivo half-life allowing for Q.I.D. dosing, preferably T.I.D. dosing, more preferably B.I.D. dosing and most preferably once-a-day dosing).

Routine assays that are well known in the art may be used to assess these properties and identify superior compounds for a particular use. For example, assays used to predict bioavailability include transport across human intestinal cell monolayers, such as Caco-2 cell monolayers. Penetration of the blood brain barrier of a compound in humans may be predicted from the brain levels of the compound in laboratory animals given the compound (e.g., intravenously). Serum protein binding may be predicted from albumin binding assays, such as those described by Oravcova, et al. (1996) Journal of Chromatography B 677:1-27. Compound half-life is inversely proportional to the required frequency of dosage. In vitro half-lives of compounds may be predicted from assays of microsomal half-life as described within Example 6, herein.

As noted above, preferred compounds provided herein are nontoxic. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria when administered at a minimum therapeutically effective amount or when contacted NK-3 receptor in vitro: (1) does not substantially inhibit cellular ATP production; (2) does not significantly prolong heart QT intervals; (3) does not cause substantial liver enlargement or (4) does not cause substantial release of liver enzymes.

As used herein, a compound that does not substantially inhibit cellular ATP production is a compound that, when tested as described in Example 7, does not decrease cellular ATP levels by more than 50%. Preferably, cells treated as described in Example 7 exhibit ATP levels that are at least 80% of the ATP levels detected in untreated cells. Highly preferred compounds are those that do not substantially inhibit cellular ATP production when the concentration of compound is at least 10-fold, 100-fold, or 1000-fold greater than the EC₅₀ or IC₅₀ for the compound.

A compound that does not significantly prolong heart QT intervals is a compound that does not result in a statistically significant prolongation of heart QT intervals (as determined by electrocardiography) in guinea pigs, minipigs, or dogs upon administration of a dose that yields a serum concentration equal to the EC₅₀ or IC₅₀ for the compound. In certain preferred embodiments, a dose of 0.01, 0.05. 0.1, 0.5, 1, 5, 10, 40, or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound does not cause substantial liver enlargement if daily treatment of laboratory rodents (e.g., mice or rats) for 5-10 days with a dose that yields a serum concentration equal to the EC₅₀ or IC₅₀ for the compound results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a compound does not promote substantial release of liver enzymes if administration of a dose that yields a serum concentration equal to the EC₅₀ or IC₅₀ for the compound does not elevate serum levels of ALT, LDH or AST in laboratory rodents by more than 3-fold (preferably no more than 2-fold) over matched mock-treated controls. In more highly preferred embodiments, such doses do not elevate such serum levels by more than 75% or 50% over matched controls. Alternately, a compound does not promote substantial release of liver enzymes if, in an in vitro hepatocyte assay, concentrations (in culture media or other such solutions that are contacted and incubated with hepatocytes in vitro) concentrations that are equal to the EC₅₀ or IC₅₀ for the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are two-fold, five-fold, and preferably ten-fold the EC₅₀ or IC₅₀ for the compound.

In other embodiments, certain preferred compounds do not inhibit or induce microsomal cytochrome P450 enzyme activities, such as CYP1A2 activity, CYP2A6 activity, CYP2C9 activity, CYP2C19 activity, CYP2D6 activity, CYP2E1 activity, or CYP3A4 activity at a concentration equal to the EC₅₀ or IC₅₀ for the compound.

Certain preferred compounds are not clastogenic or mutagenic (e.g., as determined using standard assays such as the Chinese hamster ovary cell vitro micronucleus assay, the mouse lymphoma assay, the human lymphocyte chromosomal aberration assay, the rodent bone marrow micronucleus assay, the Ames test or the like) at a concentration equal to the EC₅₀ or IC₅₀ for the compound. In other embodiments, certain preferred compounds do not induce sister chromatid exchange (e.g., in Chinese hamster ovary cells) at such concentrations.

For detection purposes, as discussed in more detail below, compounds provided herein may be isotopically-labeled or radiolabeled. For example, compounds provided herein may have one or more atoms replaced by an atom of the same element having an atomic mass or mass number different from the atomic mass or mass number usually found in nature. Examples of isotopes that can be present in the compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F, and ³⁶Cl.

### PREPARATION OF COMPOUNDS

Compounds provided herein may generally be prepared using standard synthetic methods. Starting materials are commercially available from suppliers such as Sigma-Aldrich Corp. (St. Louis, Mo.), or may be synthesized from commercially available precursors using established protocols. By way of example, a synthetic route similar to those shown in any of the following Schemes may be used, together with synthetic methods known in the art of synthetic organic chemistry, or variations thereon as appreciated by those skilled in the art. Variables in the following schemes refer to any group consistent with Formulas I herein. Where a structure contains more than one variable “R,” each R is selected independently of any other R group(s). Those skilled in the art will recognize that in certain instances it will be necessary to utilize compounds bearing protecting groups and that these groups can be removed in a subsequent reaction to yield compounds of Formula I as described in “Protective Groups in Organic Synthesis”, 2nd Ed., Greene, T. W. and related publications.

Abbreviations used in the following schemes and elsewhere herein include:
AIBN 2,2′-azobisisobutyronitrile
n-BuLi iz-butyllithium
CDCl₃ deuterated chloroform
DIEA diisopropylethylamine
DMC 2-chloro-1,3-dimethylimidazolinium chloride
DMF N.N-dimethylformamide
Et ethyl
Et₃N triethylamine
EtOAc ethyl acetate
¹H NMR proton nuclear magnetic resonance
Hz hertz
LC/MS liquid chromatography/mass spectrometry
Me methyl
MHz megahertz
MS mass spectrometry
(M+1) mass+1
NBS N-brothosuccinimide
NH₄OAc ammonium acetate
THF tetrahydrofuran
Pd(PPh₃)₄ tetrakis(triphenylphosphine) palladium (0)
PTLC preparative thin layer chromatography
SCX-SPE strong cation exchange - solid phase extraction
δ chemical shift

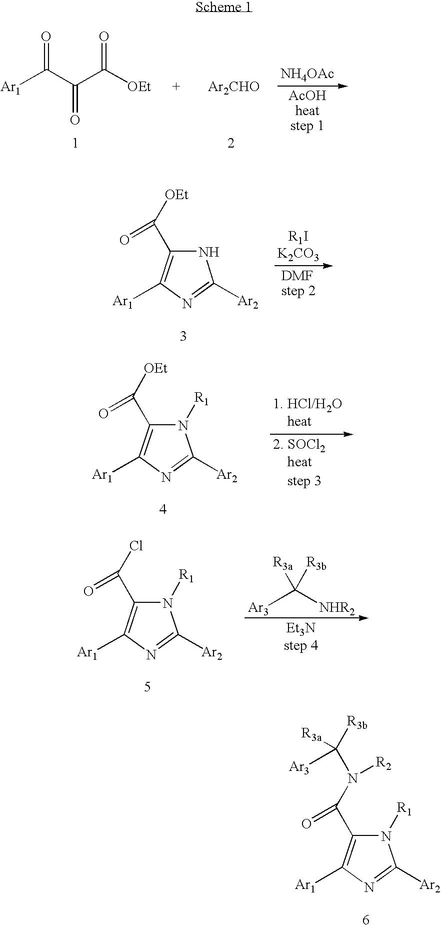

Scheme 1 illustrates a method for preparing diarylimidazoles of Formula I from the corresponding aryl tricarbonyl derivative 1 following a procedure similar to that described by Brackeen et al. (1994) Tetrahedron Letters 35(11):1635-38. Aryl tricarbonyl derivatives 1 may be obtained by a variety of literature procedures. In step 1, aryl tricarbonyl compound 1 is heated with aryl aldehyde 2 in the presence of ammonium acetate and acetic acid to obtain diaryl imidazole 3. Alkylation of 3 in step 2 yields a regioisomeric mixture of compounds including desired 1-alkyl-2,4-diarylimidazole 4. Ester hydrolysis followed by reaction with thionyl chloride in step 3 provides the corresponding acid chloride 5. In step 4, acid chloride 5 is reacted with a primary or secondary amine in the presence of a suitable base to obtain diarylimidazole 6. This route can be used to prepare a wide variety of compounds of Formula I. Those skilled in the art will recognize that alternative reagents and reaction conditions may be used for each of the steps in Scheme 1 depending on the choice or reactants and the sensitivity of functional groups present.

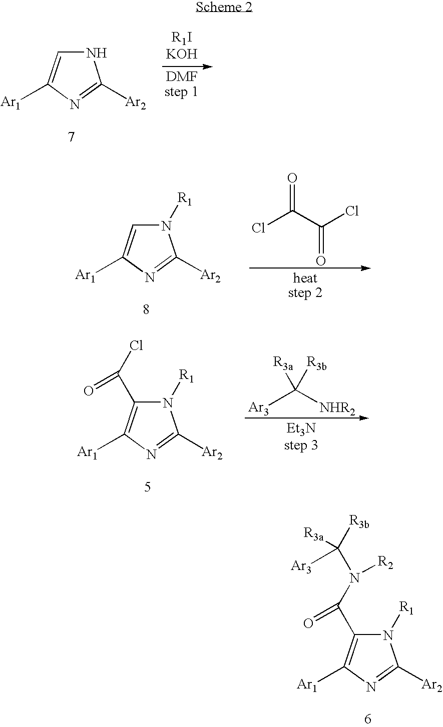

Scheme 2 employs a route adapted from the method of Chiriac (1986) Synthesis 9: 753-5 for direct carbonylation of pyrazoles. In step 1, diarylimidazole 1 is regioselectively alkylated to obtain 1-alkyl-2,4-diarylimidazole 8. In step 2, 1-alkyl-2,4-diarylimidazole 8 is heated in a stainless steel pressure reactor with a large excess of oxalyl chloride to obtain acid chloride 5. Crude acid chloride 5 can be reacted with an appropriate primary or secondary amine to obtain diarylimidazole 6. Alternatively, crude acid chloride 5 may be reacted with methanol to produce the corresponding ester. Purification of the ester produced from 5, followed hydrolysis and coupling reaction yields diarylarylimidazole 6 (e.g., as illustrated in steps 3 and 4 in Scheme 1).

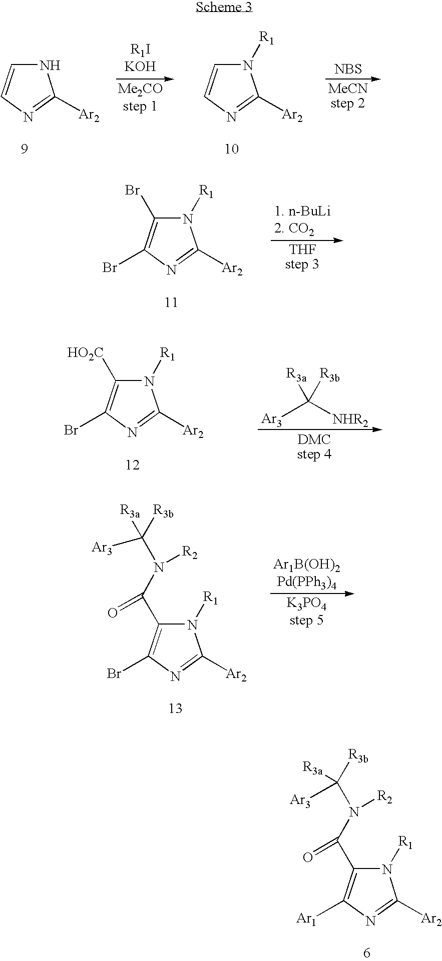

Scheme 3 illustrates a method for preparing diarylimidazoles of Formula I wherein Ar₁ is introduced via a coupling reaction. This method is useful in high-speed synthesis of a variety of diarylimidazoles in Table I. 2-Aryl imidazoles 9 are readily available from commercial sources and may be prepared by several literature methods (e.g., as described in PCT International Application Publication No. WO 02/50062). Step I involves alkylation of arylimidazole 9 with a suitable alkylating agent in the presence of base to obtain 1-alkyl-2-arylimidazole 10. Suitable alkylating agents include but are not limited to alkyl iodides, alkylbromides, and alkyl mesylates. Suitable alkylating conditions include potassium hydroxide in acetone or sodium hydride in anhydrous DMF. Depending on the choice of alkylating agent and conditions, heating may be required to facilitate the reaction. In step 2, 1-alkyl-2-arylimidazole 10 is brominated to obtain 4,5-dibromoimidazole 11. Suitable conditions for this reaction include but are not limited to treatment of 10 with 2.1 equivalents of NBS in acetonitrile. Step 3 involves regioselective metal-halogen exchange followed by reaction with carbon dioxide to obtain 4-bromo-5-carboxyimidazole 12. Suitable conditions for this transformation include treatment of 11 with a slight excess of n-BuLi in THF at −78° C. for 1 hour, followed by introduction of CO₂ gas and gradual warming to 0° C. over 3 hours. In step 4, 4-bromo-5-carboxyimidazole 12 is coupled with a primary or secondary amine to obtain the corresponding 4-bromo-5-carboxamidoimidazole 13. In step 5, 4-bromo-5-carboxamidoimidazole 13 is coupled to an appropriate metaloaryl derivative with transition metal catalysis to obtain diarylimidazole 6. For example, this may entail coupling of an arylboronic acid in the presence of palladium (0) via the Suzuki reaction (Miraura and Suzuki (1995) Chemical Reviews 95:2457).

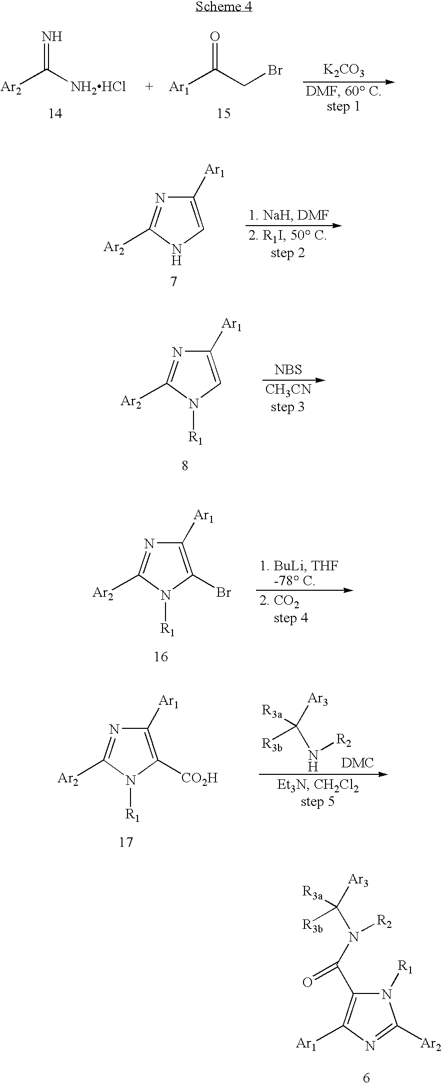

Scheme 4 illustrates an alternative route for preparing diarylimidazoles of Formula I wherein Ar₂ may be heteroaryl. In step 1, diarylimidazole 7 is prepared by reaction of an α-bromoketone 15 with amidine 14. A wide variety of α-bromoketones 15 with amidines 14 are readily available via literature methods. In step 2, 1 -alkyl-2,4-diarylimidazole 8 is prepared by regioselective alkylation of diarylimidazole 7. Step 3 entails bromination 1-alkyl-2,4-diarylimidazole 8 to obtain 5-bromoimidazole 16. Suitable brominating agents include but are not limited to NBS. In step 4, metal halogen exchange followed by reaction with gaseous carbon dioxide results in formation of the corresponding 5-carboxyimidazole 17 which is subsequently coupled with appropriate primary and secondary amines in step 5 to obtain compounds of Formula 6.

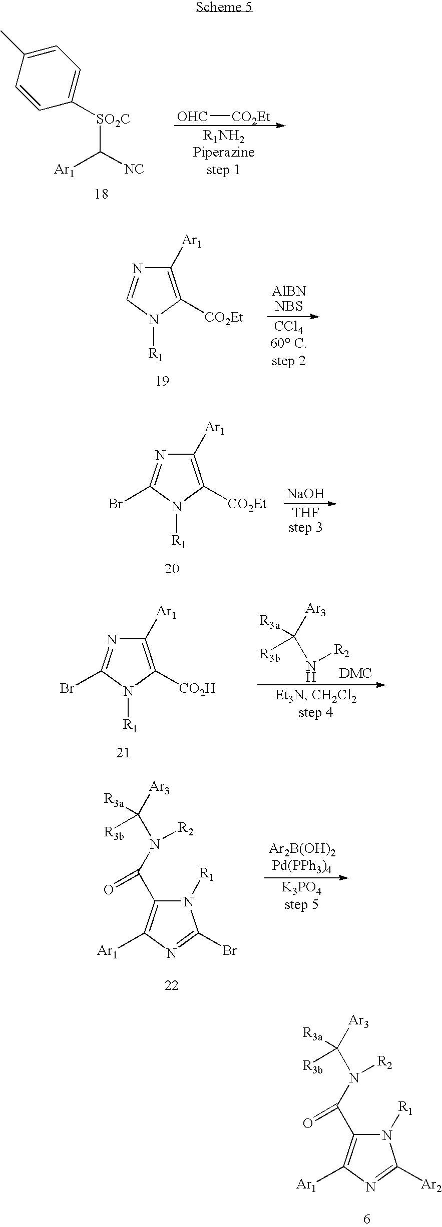

Scheme 5 illustrates a method for preparing diarylimidazoles of Formula I wherein Ar₂ is introduced via a coupling reaction. In step 1, tosylisocyanide derivative 18 is reacted with ethyl glyoxal, a suitable primary amine (R₁NH₂) and piperazine to obtain 1-alkyl-4-aryl-5-carboxyimidazole 19. Bromination of 19 is accomplished in step 2 with NBS in the presence of AIBN. The resulting 2-bromoimidazole 20 is treated with sodium hydroxide in THF to obtain the corresponding carboxylic acid 21. Step 4 entails coupling of carboxylic acid 21 with an appropriate primary or secondary amine to yield amide 22. In step 5, 2-bromo-5-carboxamidoimidazole 22 is coupled to an appropriate metaloaryl derivative with transition metal catalysis to obtain diarylimidazole 6.

In certain embodiments, a compound provided herein may contain one or more asymmetric carbon atoms, so that the compound can exist in different stereoisomeric forms. Such forms can be, for example, racemates or optically active forms. As noted above, all stereoisomers are encompassed by the present invention. Nonetheless, it may be desirable to obtain single enantiomers (i.e., optically active forms). Standard methods for preparing single enantiomers include asymmetric synthesis and resolution of the racemates. Resolution of the racemates can be accomplished, for example, by conventional methods such as crystallization in the presence of a resolving agent, or chromatography using, for example a chiral HPLC column.

Compounds may be radiolabeled by carrying out their synthesis using precursors comprising at least one atom that is a radioisotope. Each radioisotope is preferably carbon (e.g., ¹⁴C), hydrogen (e.g., ³H), sulfur (e.g., ³⁵S), or iodine (e.g., ¹²⁵I). Tritium labeled compounds may also be prepared catalytically via platinum-catalyzed exchange in tritiated acetic acid, acid-catalyzed exchange in tritiated trifluoroacetic acid, or heterogeneous-catalyzed exchange with tritium gas using the compound as substrate. In addition, certain precursors may be subjected to tritium-halogen exchange with tritium gas, tritium gas reduction of unsaturated bonds, or reduction using sodium borotritide, as appropriate. Preparation of radiolabeled compounds may be conveniently performed by a radioisotope supplier specializing in custom synthesis of radiolabeled probe compounds.

### PHARMACEUTICAL COMPOSITIONS

The present invention also provides pharmaceutical compositions comprising one or more compounds provided herein, together with at least one physiologically acceptable carrier or excipient. Pharmaceutical compositions may comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. In addition, other active ingredients may (but need not) be included in the pharmaceutical compositions provided herein.

Pharmaceutical compositions may be formulated for any appropriate manner of administration, including, for example, topical, oral, nasal, rectal or parenteral administration. The term parenteral as used herein includes subcutaneous, intradermal, intravascular (e.g., intravenous), intramuscular, spinal, intracranial, intrathecal, and intraperitoneal injection, as well as any similar injection or infusion technique. In certain embodiments, compositions suitable for oral use are preferred. Such compositions include, for example, tablets, troches, lozenges, aqueous or oily suspensions, dispersible powders or granules, emulsion, hard or soft capsules, or syrups, or elixirs. Within yet other embodiments, compositions of the present invention may be formulated as a lyophilizate. Formulation for topical administration may be preferred for certain conditions.

Compositions intended for oral use may further comprise one or more components such as sweetening agents, flavoring agents, coloring agents and/or preserving agents in order to provide appealing and palatable preparations. Tablets contain the active ingredient in admixture with physiologically acceptable excipients that are suitable for the manufacture of tablets. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffm or olive oil).

Aqueous suspensions contain the active material(s) in admixture with excipients suitable for the manufacture of aqueous suspensions. Such excipients include suspending agents (e.g., sodium carboxymethylcellulose, methylcellulose, hydropropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also comprise one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredient(s) in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. Sweetening agents such as those set forth above, and/or flavoring agents may be added to provide palatable oral preparations. Such suspensions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavoring and coloring agents, may also be present.

Pharmaceutical compositions may also be formulated as oil-in-water emulsions. The oily phase may be a vegetable oil (e.g., olive oil or arachis oil), a mineral oil (e.g., liquid paraffin) or a mixture thereof. Suitable emulsifying agents include naturally-occurring gums (e.g., gum acacia or gum tragacanth), naturally-occurring phosphatides (e.g., soy bean lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). An emulsion may also comprise one or more sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

Formulations for topical administration typically comprise a topical vehicle combined with active agent(s), with or without additional optional components. Suitable topical vehicles and additional components are well known in the art, and it will be apparent that the choice of a vehicle will depend on the particular physical form and mode of delivery. Topical vehicles include water; organic solvents such as alcohols (e.g., ethanol or isopropyl alcohol) or glycerin; glycols (e.g., butylene, isoprene or propylene glycol); aliphatic alcohols (e.g., lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); and hydrocarbon-based materials such as microsponges and polymer matrices. A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatin-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids and emulsions. The physical appearance and viscosity of such pharmaceutically acceptable forms can be governed by the presence and amount of emulsifier(s) and viscosity adjuster(s) present in the formulation. Solids are generally firm and non-pourable and commonly are formulated as bars or sticks, or in particulate form; solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels and often do not contain emulsifiers. Liquid topical products often contain solvents, emulsifiers, moisturizers, emollients, fragrances, dyes/colorants, preservatives and other active ingredients that increase or enhance the efficacy of the final product.

Typical modes of delivery for topical compositions include application using the fingers; application using a physical applicator such as a cloth, tissue, swab, stick or brush; spraying (including mist, aerosol or foam spraying); dropper application; sprinkling; soaking; and rinsing. Controlled release vehicles can also be used.

A pharmaceutical composition may be prepared as a sterile injectible aqueous or oleaginous suspension. The compound of Formula I, depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectible compositions, and adjuvants such as local anesthetics, preservatives and/or buffering agents can be dissolved in the vehicle.

Compounds may also be formulated as suppositories (e.g., for rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions may be formulated as sustained release formulations (i.e., a formulation such as a capsule that effects a slow release of compound following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of compound release. The amount of compound contained within a sustained release formulation depends upon, for example, the site of implantation, the rate and expected duration of release and the nature of the condition to be treated or prevented.

In addition to, or together with, the above modes of administration, a compound of Formula I may be conveniently added to food or drinking water (e.g., for administration to non-human animals including companion animals (such as dogs and cats) and livestock). Animal feed and drinking water compositions may be formulated so that the animal takes in an appropriate quantity of the composition along with its diet. It may also be convenient to present the composition as a premix for addition to feed or drinking water.

Compounds provided herein are generally present within a pharmaceutical composition in a therapeutically effective amount, as described above. Compositions providing dosage levels ranging from about 0.1 mg to about 140 mg per kilogram of body weight per day are preferred (about 0.5 mg to about 7 g per human patient per day), with oral doses generally being about 5-20 fold higher than intravenous doses (e.g., ranging from 0.01 to 40 mg per kilogram of body weight per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. It will be understood, however, that the optimal dose for any particular patient will depend upon a variety of factors, including the activity of the specific compound employed; the age, body weight, general health, sex and diet of the patient; the time and route of administration; the rate of excretion; any simultaneous treatment, such as a drug combination; and the type and severity of the particular disease undergoing treatment. Optimal dosages may be established using routine testing and procedures that are well known in the art.

Pharmaceutical compositions may be packaged for treating conditions responsive to NK-3 receptor modulation (e.g., treatment of psychosis, schizophrenia, depression, chronic pulmonary obstructive disorder or other disease or disorder as described herein). Packaged pharmaceutical compositions may include a container holding a therapeutically effective amount of at least one compound described herein and instructions (e.g., labeling) indicating that the contained composition is to be used for treating a condition responsive to NK-3 receptor modulation in the patient.

### METHODS OF USE

Compounds provided herein may be used to alter activity and/or activation of NK-3 receptors in a variety of contexts, both in vitro and in vivo. Within certain aspects, NK-3 receptor antagonists may be used to inhibit the binding of NK-3 receptor agonist (such as neurokinin) to NK-3 receptor in vitro or in vivo. In general, such methods comprise the step of contacting a NK-3 receptor with one or more compounds provided herein in the presence of NK-3 receptor ligand in aqueous solution and under conditions otherwise suitable for binding of the ligand to NK-3 receptor. The NK-3 receptor may be present in solution or suspension (e.g., in an isolated membrane or cell preparation), or in a cultured or isolated cell. Within certain embodiments, the NK-3 receptor is expressed by a neuronal cell present in a patient, and the aqueous solution is a body fluid. Preferably, one or more NK-3 modulators are administered to an animal in an amount as described above.

Also provided herein are methods for modulating, preferably inhibiting, the signal-transducing activity of a NK-3 receptor. Such modulation may be achieved by contacting a NK-3 receptor (either in vitro or in vivo) with a one or more compounds provided herein under conditions suitable for binding of the compound(s) to the receptor. The receptor may be present in solution or suspension, in a cultured or isolated cell preparation or within a patient. Modulation of signal tranducing activity may be assessed by detecting an effect on calcium ion conductance (also referred to as calcium mobilization or flux). Modulation of signal transducing activity may alternatively be assessed by detecting an alteration of a symptom of a patient being treated with one or more compounds provided herein.

The present invention further provides methods for treating conditions responsive to NK-3 receptor modulation. Within the context of the present invention, the term “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms). A condition is “responsive to NK-3 receptor modulation” if it is characterized by inappropriate activity of a NK-3 receptor, regardless of the amount of NK-3 receptor ligand present locally, and/or if modulation of NK-3 receptor activity results in alleviation of the condition or a symptom thereof. Such conditions include, for example, anxiety, depression, psychosis, obesity, chronic pulmonary obstructive disorder, gastrointestinal conditions such as irritable bowel syndrome or colitis, pain and cognitive disorders as described herein. Such conditions may be diagnosed and monitored using criteria that have been established in the art. Patients include humans, domesticated companion animals and livestock, with dosages as described above.

Also provided herein are methods for using diaryl imidazoles of Formula I to treat a condition responsive to canriabinoid receptor (especially CB1) modulation in a patient. The patient may be afflicted with such a condition, or may be considered at risk for developing such a condition. A condition is “responsive to CB1 modulation” if the condition or symptom(s) thereof are alleviated, attenuated, delayed or otherwise improved by modulation of CB1 activity. Such conditions include, for example, appetite disorders, obesity, addictive disorders, asthma, liver cirrhosis, sepsis, irritable bowel disease, Crohn's disease, depression, memory disorders, cognitive disorders and movement disorders. Methods are further provided herein for appetite suppression. In general, methods for treating such conditions comprise administering to the patient a therapeutically effective amount of at least one compound according to Formula I.

It will be apparent that compounds provided herein may be administered alone or in combination with one or more additional agents that are suitable for treating the disorder of interest. Within combination therapy, the compound(s) of Formula I and additional agent(s) may be present in the same pharmaceutical composition, or may be administered separately in either order. Representative additional agents are as described above.

Suitable dosages for compounds provided herein within such combination therapy are generally as described above. Dosages and methods of administration of the additional agent(s) can be found, for example, in the manufacturer's instructions or in the Physician's Desk Reference. In certain embodiments, combination administration results in a reduction of the dosage of the additional agent required to produce a therapeutic effect (i.e., a decrease in the minimum therapeutically effective amount). Thus, preferably, the dosage of additional agent in a combination or combination treatment method of the invention is less than the maximum dose advised by the manufacturer for administration of the agent without combination with a compound of Formula I. More preferably this dose is less than ¾, even more preferably less than ½, and highly preferably, less than ¼ of the maximum dose, while most preferably the dose is less than 10% of the maximum dose advised by the manufacturer for administration of the agent(s) when administered without combination administration as described herein. It will be apparent that the dose of compound of Formula I needed to achieve the desired effect may similarly be affected by the dose and potency of the additional agent.

In certain preferred embodiments, the combination administration is accomplished by packaging one or more compounds provided herein and one or more additional agents in the same package, either in separate containers within the package or in the same container as a mixture. Preferred mixtures are formulated for oral administration (e.g., as pills, capsules, tablets or the like). In certain embodiments, the package comprises a label bearing indicia indicating that the components are to be taken together for the treatment of anxiety, depression, schizophrenia, psychosis, chronic pulmonary obstructive disorder, irritable bowel syndrome, colitis, pain, an appetite disorder, obesity or an addictive disorder.

Administration to the patient can be by way of any means discussed above, including oral, topical, nasal or transdermal administration, or intravenous, intramuscular, subcutaneous, intrathecal, epidural, intracerebroventrilcular or like injection. Oral administration is preferred in certain embodiments (e.g., formulated as pills, capsules, tablets or the like).

Treatment regimens may vary depending on the compound used and the particular condition to be treated. However, for treatment of most disorders, a frequency of administration of 4 times daily or less is preferred. In general, a dosage regimen of 2 times daily is more preferred, with once a day dosing particularly preferred. It will be understood, however, that the specific dose level and treatment regimen for any particular patient will depend upon a variety of factors including the activity of the specific compound employed, the age, body weight, general health, sex, diet, time of administration, route of administration, and rate of excretion, drug combination and the severity of the particular disease undergoing therapy. In general, the use of the minimum dose sufficient to provide effective therapy is preferred. Patients may generally be monitored for therapeutic effectiveness using medical or veterinary criteria suitable for the condition being treated or prevented.

Within separate aspects, the present invention provides a variety of non-pharmaceutical in vitro and in vivo uses for the compounds provided herein. For example, such compounds may be labeled and used as probes for the detection and localization of NK-3 receptor (in samples such as cell preparations or tissue sections, preparations or fractions thereof). Compounds may also be used as positive controls in assays for receptor activity, as standards for determining the ability of a candidate agent to bind to NK-3 receptor, or as radiotracers for positron emission tomography (PET) imaging or for single photon emission computerized tomography (SPECT). Such methods can be used to characterize NK-3 receptors in living subjects. For example, a compound provided herein may be labeled using any of a variety of well known techniques (e.g., radiolabeled with a radionuclide such as tritium, as described herein), and incubated with a sample for a suitable incubation time (e.g., determined by first assaying a time course of binding). Following incubation, unbound compound is removed (e.g., by washing), and bound compound detected using any method suitable for the label employed (e.g., autoradiography or scintillation counting for radiolabeled compounds; spectroscopic methods may be used to detect luminescent groups and fluorescent groups). As a control, a matched sample containing labeled compound and a greater (e.g., 10-fold greater) amount of unlabeled compound may be processed in the same manner. A greater amount of detectable label remaining in the test sample than in the control indicates the presence of NK-3 receptor in the sample. Detection assays, including receptor autoradiography (receptor mapping) of NK-3 receptor in cultured cells or tissue samples may be performed as described by Kuhar in sections 8.1.1 to 8.1.9 of Current Protocols in Pharmacology (1998) John Wiley & Sons, New York.

Compounds provided herein may also be used within a variety of well-known cell separation methods. For example, such compounds may be linked to the interior surface of a tissue culture plate or other support, for use as affinity ligands for immobilizing and thereby isolating, NK-3 receptors (e.g., isolating receptor-expressing cells) in vitro. Within one preferred embodiment, a compound linked to a fluorescent marker, such as fluorescein, is contacted with the cells, which are then analyzed (or isolated) by fluorescence activated cell sorting (FACS).

The following Examples are offered by way of illustration and not by way of limitation. Unless otherwise specified all reagents and solvent are of standard commercial grade and are used without further purification. Using routine modifications, the starting materials may be varied and additional steps employed to produce other compounds provided herein.

### EXAMPLES

LC/MS data provided herein is obtained by the following method:

Analytical HPLC/MS instrumentation: Analyses are performed using a Waters 600 series pump (Waters Corporation, Milford, Mass.), a Waters 996 Diode Array Detector and a Gilson 215 auto-sampler (Gilson Inc, Middleton, Wis.), Micromass™ LCT time-of-flight electrospray ionization mass analyzer. Data are acquired using MassLynx™ 4.0 software, with OpenLynx Global Server™, OpenLynx™, and AutoLynx™ processing.

Analytical HPLC conditions: 4.6×50 mm, Chromolith SpeedROD RP-18e column (Merck KGaA, Darmstadt, Germany); UV 10 spectra/sec, 220-340 nm summed; flow rate 6.0 mL/min; injection volume 1 μl;

Gradient conditions—mobile phase A is 95% water, 5% methanol with 0.05% TFA; mobile phase B is 95% methanol, 5% water with 0.025% TFA, and the gradient is 0-0.5 minutes 10-100% B, hold at 100% B to 1.2 minutes, return to 10% B at 1.21 minutes. Inject-to-inject cycle time is 2.15 minutes.

Analytical MS conditions: capillary voltage 3.5 kV; cone voltage 30V; desolvation and source temperature are 350° C. and 120° C., respectively; mass range 181-750 with a scan time of 0.22 seconds and an inter scan delay of 0.05 minutes.

### Example 1

### Preparation of Representative Diaryl Imidazole Derivatives

### 1-1. (S)-3-METHYL-2,5-DIPHENYL-3H-IMIDAZOLE-4-CARBOXYLIC ACID (1-Phenyl-Propyl)-amdie

a. 2,5-Diphenyl-3H-imidazole-4-carboxylic acid ethyl ester

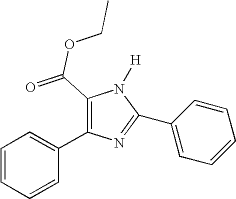

A mixture of 2,3-dioxo-3-phenyl-propionic acid ethyl ester (24.6 mmol), benzaldehyde (30 mmol), ammonium acetate (200 mmol) and acetic acid (100 mL) is stirred under nitrogen at 50° C. for 2.5 hours. Acetic acid is removed under vacuum and the residue is partitioned between ethyl acetate (200 mL)/water (200 mL) and treated with 1 M sodium carbonate solution until the aqueous phase reaches pH 8. The aqueous phase is removed and the ethyl acetate layer is dried over anhydrous magnesium sulfate, filtered, and evaporated. The residue is purified by chromatography on silica gel (2% ether/chloroform) to obtain 2,5-diphenyl-3H-imidazole-4-carboxylic acid ethyl ester as a tan solid.
b. 3-Methyl-2,5-diphenyl-3H-imidazole-4-carboxylic acid ethyl ester

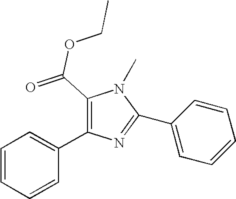

A mixture of 2,5-diphenyl-3H-imidazole-4-carboxylic acid ethyl ester (2.8 mmol), methyl iodide (4.0 mmol), potassium carbonate (6.0 mmol), and dimethylformamide (DMF; 15 mL) is stirred vigorously at room temperature for 2.5 hours. The mixture is poured into water (200 mL) and extracted with ethyl acetate (50 mL). The ethyl acetate layer is washed with water (50 mL), dried over anhydrous magnesium sulfate, filtered and evaporated. The residue is purified by chromatography on silica gel (2% ether/chloroform) to obtain 3-methyl-2,5-diphenyl-3H-imidazole-4-carboxylic acid ethyl ester. ¹³C NMR (400 MHz, CDCl₃) δ=13.74, 13.76, 34.84, 60.39, 120.00, 127.37, 127.84, 128.47, 128.51, 128.46, 128.52, 129.59, 134.457, 148.55, 151.23, 161.09. Isomeric 1-methyl-2,5-diphenyl-1H-imidazole-4-carboxylic acid ethyl ester is obtained as a minor product.
c. (S)-3-Methyl-2,5-diphenyl-3H-imidazole-4-carboxylic acid (1-phenyl-propyl)-amide

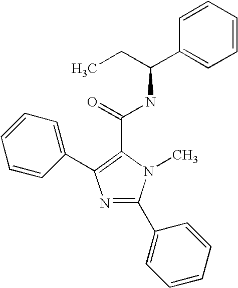

A mixture of 3-methyl-2,5-diphenyl-3H-imidazole-4-carboxylic acid ethyl ester and concentrated hydrochloric acid (1 mL) is stirred and heated at reflux for 16 hours. The mixture is evaporated at reduced pressure and the residue is stirred with thionyl chloride (1 mL) at 80° C. for 30 minutes. The mixture is concentrated at reduced pressure to obtain 3-methyl-2,5-diphenyl-3H-imidazole-4-carbonyl chloride which may be used without fuirther purification. 3-Methyl-2,5-diphenyl-3H-imidazole-4-carbonyl chloride is treated with (S)-1-phenylpropylamine (0.5 mL) and anhydrous triethylamine (3 mL) and the mixture is stirred at 80° C. under nitrogen for 30 minutes. The reaction mixture is poured into water (50 mL), acidified to pH 6 using 1 M hydrochloric acid and extracted with chloroform (20 mL). The extract is dried over anhydrous magnesium sulfate, filtered and evaporated. The residue so obtained is purified by chromatography on silica gel (5% ether/chloroform) to obtain the title compound. ¹³C NMR (400 MHz, CDCl₃) δ=10.45, 28.91, 34.51, 55.10, 55.15, 123.54, 126.52, 128.52, 128.66, 128.71, 129.34, 129.75, 134.01, 141.32, 142.96, 150.10, 160.38.

The following compounds are prepared by the method illustrated in Example 1-1, step c, starting with 3-methyl-2,5-diphenyl-3H-imidazole-4-carbonyl chloride and the appropriate amine:

### 1-2. 3-METHYL-2,5-DIPHENYL-3H-IMIDAZOLE-4-CARBOXYLIC ACID (1 -PHENYL-BUTYL)-AMIDE

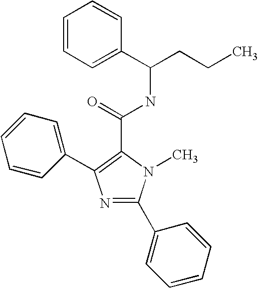

### 1-3. 3 -METHYL-2,5-DIPHENYL-3H-IMIDAZOLE-4-CARBOXYLIC ACID N′-METHYL-N′-PHENYL-HYDRAZIDE

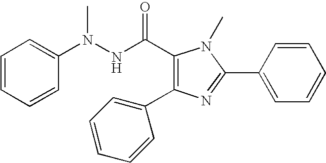 ¹H NMR (CDCl₃) δ 3.15 (s, 3H), 3.89 (s, 3H), 6.74-6.86 (m, 3H), 7.19-7.25 (m, 2H), 7.40-7.67 (m, 6H), 7.64-7.69 (m, 4H).

### 1-4. METHYL-2,5-DIPHENYL-3H-IMIDAZOLE-4-CARBOXYLIC ACID N′-ETHYL-N′-PHENYL-HYDRAZIDE

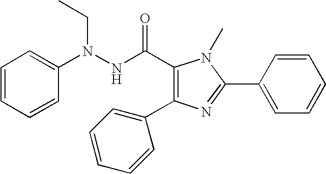 ¹H NMR (CDCl₃) δ 1.01 (t, 3H), 3.54 (q, 2H), 3.95 (s, 3H), 6.74-6.86 (m, 3H), 7.19-7.25 (m, 2H), 7.40-7.67 (m, 6H), 7.64-7.69 (m, 4H).

### 1-5. 3-METHYL-2,5-DIPHENYL-3H-IMIDAZOLE-4-CARBOXYLIC ACID (3-HYDROXY-1-PHENYL-PROPYL)-AMIDE

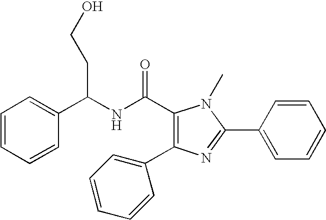 ¹H NMR (CDCl₃) δ 1.62-1.72 (m, 1H), 1.86-2.00 (m, 1H), 3.12 (t, 1H), 3.57 (m, 2H), 3.91 (s,3H), 5.20-5.28 (m,1H), 6.30 (d, 1H), 6.96-7.00 (m, 2H), 7.24-7.32 (m, 6H), 7.45-7.54 (m, 5H), 7.62-7.65 (m, 2H).

### 1-6. 3-METHYL-2,5-DIPHENYL-3H-IMIDAZOLE-4-CARBOXYLIC ACID (2-HYDROXY-2-METHYL-1-PHENYL-PROPYL)-AMIDE

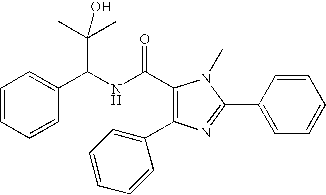 ¹H NMR (CDCl₃) δ 0.93 (s, 3H), 1.15 (s, 3H), 3.88 (s,3H), 4.89 (d, 1H), 6.72 (d, 1H), 6.98-7.01 (m, 2H), 7.23-7.26 (m, 3H), 7.39-7.45 (m, 6H), 7.56-7.64 (m, 4H).

### 1-7. 3-METHYL-2,5-DIPHENYL-3H-IMIDAZOLE-4-CARBOXYLIC ACID (3-HYDROXY-3-METHYL-1-PHENYL-BUTYL)-AMIDE

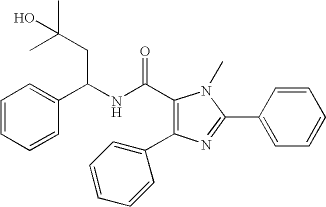 ¹H NMR (CDCl₃) δ 1.14 (s, 3H), 1.24 (s, 3H), 1.73-1.86 (m, 2H), 1.93 (s, 1H), 3.88 (s, 3H), 5.01-5.17 (m, 1H), 6.74 (d, 1H), 7.08-7.11 (m, 2H), 7.23-7.38 (m, 6H), 7.44-7.50 (m, 3H), 7.56-7.64 (m, 4H).

### 1-8. 3-METHYL-2,5-DIPHENYL-3H-IMIDAZOLE-4-CARBOXYLIC ACID ((S)-1-CYCLOHEXYL-ETHYL)-AMIDE

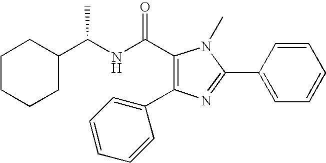 ¹H NMR (CDCl₃) δ 0.60-0.72 (m, 1H), 0.81-0.92 (m, IH), 0.96 (d, 3H), 1.01-1.20 (m, 4H), 1.38 (d, 1H), 1.51-1.71 (m, 5H), 3.92 (s,3H), 5.58 (d, 1H), 7.25-7.50 (m, 6 H), 7.60-7.66 (m, 4H).

### Example 2

### Preparation of Additional Representative Diaryl Imidazole Derivatives

### 2-1. 3 -METHYL-2,5 -DIPHENYL-3H-IMIDAZOLE-4-CARBOXYLIC ACID (1 -PHENYL-ALLYL)-AMIDE

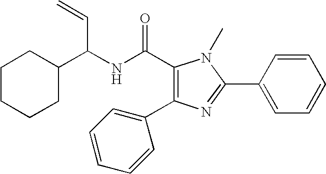

[Bis(2-methoxyethyl)-amino]sulfur trifluoride (0.2 mL) is added to a solution of 3-methyl-2,5-diphenyl-3H-imidazole-4-carboxylic acid (3-hydroxy-1-phenyl-propyl)-amide [Example 1, 1-5] (40 mg, 0.9 mmol) in DCM (5 mL) at −78° C. After stirring for one hour at −78° C., the mixture is allowed to warm to room temperature. The mixture is neutralized with saturated NaHCO₃ and extracted with DCM. The organic layers are dried and solvent evaporated. The residue is purified by PTLC with 5% methanol in DCM to give the desired compound. ¹H NMR (CDCl₃) δ 1.94-2.10 (m, 1H), 2.30-2.40 (m, 1H), 3.85 (s,3H), 4.22-4.34 (m, 2H), 4.83 (dd, 1H), 7.25-7.50 (m, 11H), 7.64-7.67 (m, 2H), 7.76-7.79 (m, 2H).

### 2-2. 3-ETHYL-2,5-DIPHENYL-3H-IMfDAZOLE-4-CARBOXYLIC ACID (1,2,3,4-TETRAHYDRO-NAPHTHALEN-1-YL)-AMIDE

a. 1-Ethyl-2,4-diphenyl-1H-imidazole

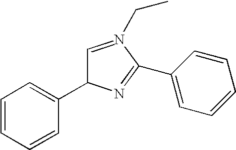

50% aqueous KOH (40 mmol) and iodoethane (40 mmol) are added to a solution of 2,4-diphenyl-1H-imidazole (30 mmol) in DMF (50 mL), and the mixture is stirred at 40° C. under nitrogen for 16 hours. The reaction mixture is partitioned between water (300 mL) and ethyl acetate (200 mL). The organic layer is separated, washed with water (200 mL×2), dried over anhydrous magnesium sulfate, filtered and evaporated at reduced pressure. The residue is dissolved in xylenes (100 mL) and evaporated at reduced pressure. Chromatography on silica gel (chloroform) provides pure 1-ethyl-2,4-diphenyl-1H-imidazole. ¹³C NMR (400 MHz, CDCl₃) δ=16.16, 41.43, 115.44, 124.57, 126.36, 128.26, 128.32, 128.54, 128.68, 130.55, 134.04, 140.87, 147.48.
b. 3-Ethyl-2,5-diphenyl-3H-imidazole-4-carboxylic acid (1,2,3,4-tetrahydro-naphthalen-1-yl)-amide

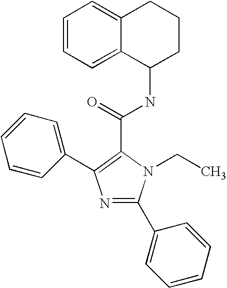

A mixture of 1-ethyl-2,4-diphenyl-1H-imidazole (1.32 g) and oxalyl chloride (3 mL) is heated in a sealed Teflon-lined reactor at 150° C. for 5 hours, and then allowed to cool and stand at room temperature for 16 hours. Volatiles are removed at reduced pressure and the residue is used in the next step without further purification. A portion of the residue from above (˜40%) is treated with 1-amino-1,2,3,4-tetrahydronaphthalene (0.5 mL) and triethylamine (3 mL) and the resulting mixture is heated at reflux for 15 minutes. The mixture is partitioned between water (40 mL) and chloroform (40 mL) and the pH of the aqueous phase is adjusted to ˜7 using 1 N HCl. The organic phase is separated, dried over anhydrous magnesium sulfate, filtered and evaporated. Chromatography on silica gel (2:1 hexanes/ether) provides 3-ethyl-2,5-diphenyl-3H-imidazole-4-carboxylic acid (1,2,3,4-tetrahydro-naphthalen-1-yl)-amide. LCMS M+H =422.2.

### 2-3. GENERAL METHOD FOR PREPARATION OF 1-ALKYL-2,4-DDIARYLIMIDAZOL-5-CARBOXAMIDES (SCHEME 3)

a. 1-Ethyl-2-phenyl-1H-imidazole

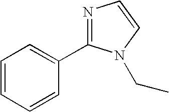

Freshly powdered KOH (14 g, 250 mmol), and then acetone (200 mL) are added to a reaction vessel. 2-phenylimidazole (7.2 g, 50 mmol) is added to the resulting suspension and the reaction is stirred 0.5 hours under N₂. Ethyliodide (8.58 g, 55 mmol) is then added and the reaction is stirred at room temperature. The reaction is monitored via LCMS at 0.5 hour intervals; if 2-phenylimidazole is still present additional aliquots of ethyliodide (0.86 g, 5.5 mmol) are added. Once the starting material is completely consumed, the reaction is concentrated to half volume in vacuo, and then diluted with ether and transferred to a separatory funnel where the organic layer is washed with water. The aqueous wash is removed and reextracted with ether; the combined organic extracts are then washed with saturated NaCl. The organic layer is extracted twice with HCl (2N), and the combined acidic aqueous extracts are washed with DCM. The aqueous solution is basified with NaOH (2.5 M) and extracted with EtOAc. The organic extract is dried over Na₂SO₄, filtered and concentrated in vacuo.
b. 4,5-Dibromo-1-ethyl-2-phenyl-1H-imidazole

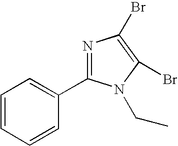

1-Ethyl-2-phenyl-1H-imidazole (8.6 g, 50 mmol) is dissolved in acetonitrile (250 mL). The reactor is placed in a water bath (20° C.). NBS (2×9.8 g, 110 mmol total) is added batchwise with a 20 minute interval between additions. The reaction is allowed to stir at bath temperature for 1 hour. The reaction is concentrated to half volume in vacuo. The resulting solution is diluted with ether and transferred to a separatory funnel where it is washed with a mixture of saturated NaHCO₃ and saturated Na₂SO₃ (3:1). The aqueous wash is reextracted with ether, and the combined organic extracts are washed with brine then dried over Na₂SO₄, filtered and concentrated in vacuo. The resulting dark oil is purified on a SiO₂ column eluted with 10% ethyl acetate/hexanes. The 0.35 rf (10% ethyl acetate/hexanes) component is collected and concentrated in vacuo.
c. 5-Bromo-4-carboxy-1-ethyl-2-phenyl-1H-imidazole

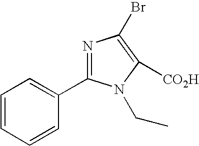

Under a nitrogen atmosphere, 4,5-dibromo-1-ethyl-2-phenyl-1H-imidazole (12.7 g, 38.4 mmol) is dissolved in THF (250 mL, anhydrous). The reactor is brought to −78° C. and n-BuLi (20 mL, 2.0 M in cyclohexane, 40 mmol) is added over 20 minutes. The reaction is stirred for 1 hour at −78° C.; carbon dioxide is then bubbled through the solution as it is allowed to slowly warm to 0° C. over 3 hours. The reaction is kept at 0° C. for 0.5 hours, and then quenched with ice and allowed to come to room temperature. The reaction is concentrated to half volume in vacuo, and then diluted with ether. The resulting solution is extracted with NaOH (1 N) and the extract is washed with ether. The basic aqueous solution is then acidified with HBr (2 N) to pH 4 and extracted with ethyl acetate, and the organic extract is dried over Na₂SO₄, filtered and concentrated in vacuo. The resulting semi-solid is triturated with Et₂O and concentrated in vacuo to produce the title compound as a tan powder.
d. General methodfor preparation of 1-ethyl-2,4-diarylimidazol-5-carboxyamides

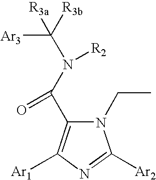

1-Ethyl-2-aryl-4-bromoimidazol-5-carboxylic acid, such as 5-bromo-4-carboxy-1-ethyl-2-phenyl-1H-imidazole, (0.2 M in toluene with 5% DIEA; 0.100 mL; 0.020 mmol) is added to a reaction vessel followed by an amine (0.2 M in toluene; 0.110 mL; 0.022 mmol) and 2-chloro-1,3-dimethylimidazolinium chloride (0.2 M in acetonitrile; 0.110 mL; 0.022 mmol). The reaction vessel is allowed to stand at room temperature for 2 hours, and then is treated with potassium phosphate (tribasic, 1.0 M, 0.100 mL; 0.100 mmol), arylboronic acid (0.2 M in 1,4-dioxane; 0.150 mL, 0.030 mmol) and, under a nitrogen atmosphere, Pd(PPh₃)₄ (0.01 M in toluene, 0.050 mL; 0.0005 mmol). The reaction is heated to 80° C. under a nitrogen atmosphere for 14 hours, and then treated with saturated NaHCO₃ (0.5 mL) and ethyl acetate (0.3 mL). The organic layer is purified via SCX SPE. By replacing ethyl iodide with other alkylating agents (e.g., methyl iodide), R₁ is varied to prepare additional compounds of Formula I.

### 2-4. 1-ETHYL-2-PHENYL-N-[(1S)-1-PHENYLPROPYL]-4-HETEROARYL-1H-IMIDAZOLE-5-CARBOXAMIDE DERIVATIVES (SCHEME 3)

A. 1-Ethyl-2-phenyl-N-[(1S)-1-phenylpropyl]-4-pyrazin-2-yl-1H-imidazole-5-carboxamide

a. 4-Bromo-1-ethyl-2-phenyl-N-[(1S)-1-phenylpropyl]-1H-imidazole-5-carboxamide

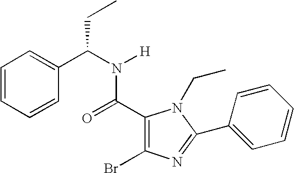

Et₃N (0.51 ml, 3.64 mmol) is added dropwise to a solution of 4-bromo-1-ethyl-2-phenyl-1H-imidazole-5-carboxylic acid (537 mg, 1.82 mmol), (1S)-1-phenylpropan-1-amine (246 mg, 1.82 mmol) and DMC (308 mg, 1.82 mmol) in CH₂Cl₂ (5 ml). The resulting yellow suspension is stirred at ambient temperature for 6 hours. The mixture is diluted with CH₂Cl₂ (10 ml) and washed with brine (10 ml), dried (Na₂SO₄) and solvent evaporated in vacuo. Flash column chromatography separation of the residue with 5:1 hexanes/EtOAc gives the title compound as white solid.
b. 1-Ethyl-2-phenyl-N-[(1S)-1-phenylpropyl]-4-pyrazin-2-yl-1H-imidazole-5-carboxamide

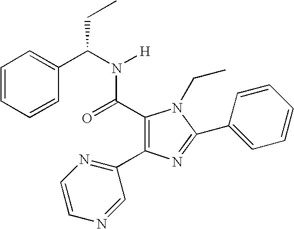

A mixture of 4-bromo-1-ethyl-2-phenyl-N-[(1S)-1-phenylpropyl]-1H-imidazole-5-carboxamide (51 mg, 0.124 mmol), Pd(PPh₃)₄ (12 mg, 0.01 mmol) and 2-tributylstannylpyrazine (70 mg, 0.19 mmol) in toluene (5 ml) is bubbled with argon for 5 minutes, and then stirred at 110° C. in a sealed tube for 16 hours. Saturated KF aqueous solution (5 ml) is added and the mixture is vigorously stirred at ambient temperature for 1 hour. The layers are separated and the aqueous layer is extracted with EtOAc (5 ml). The combined extracts are washed with brine (5 ml), dried (Na₂SO₄), and the solvent evaporated in vacuo. PTLC separation of the residue with pure EtOAc gives the title compound as a white solid. LC-MS (M+1) 412.08; ¹H-NMR (δ, CDCl₃) 11.45 (d, 1H), 9.57 (d, 1H), 8.52 (d, 1H), 8.22 (t, 1H), 7.50-7.61 (m, 5H), 7.26-7.39 (m, 5H), 5.03 (q, 1H), 4.43-4.66 (m, 2H), 1.89-2.11 (m, 2H), 1.31 (t, 3H), 0.95 (t, 3H).
B. 1-Ethyl-2-Phenyl-N-[(1S)-1-Phenylpropyl]-4-(1,3-Thiazol-2-yl)-1H-Imidazole-5-Carboxamide

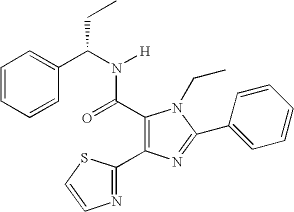

This compound is prepared via procedure analogous to that described above. LC-MS (M+1) 417.03; ¹H-NMR (δ, CDCl₃) 12.07 (d, 1H), 7.79 (d, 1H), 7.24-7.59 (m, 11H), 5.09 (q, 1H), 4.44-4.67 (m, 2H), 1.87-2.12 (m, 2H), 1.31 (t, 3H), 0.99 (t, 3H).

### 2-5. 3-METHYL-5-PHENYL-2-HETEROARYL-3H-IMIDAZOLE-4-CARBOXYLIC ACID ((S)-1-PHENYL-PROPYL)-AMIDES (SCHEME 4)

A. 3-Methyl-5-phenyl-2-pyridin-3-yl-3H-imidazole-4-carboxylic acid ((S)-1-phenyl-propyl)-amide

a. 3-(4-Phenyl-1H-imidazol-2-yl)-pyridine

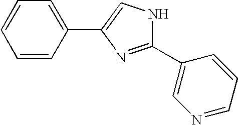

To a solution of pyridine-3-carboximidamide hydrochloride (5 g, 31.7 mmol) in anhydrous DMF (25 mL) is added potassium carbonate (10.9 g, 2.5 eq.), followed by the addition of a solution of 2-bromoacetophenone (5.39 g, 27.1 mmol) in DMF (30 mL) dropwise at 55° C. The resulting mixture is stirred at 60° C. for an additional 3 hours. Upon cooling to room temperature, the reaction mixture is poured into water (100 mL), and extracted with dichloromethane (50 mL×3). The organic layers are washed with water (30 mL×4) and brine, dried over sodium sulfate, and solvent evaporated. The residue is purified by flash chromatography (CH₂Cl₂/MeOH, 10:1) to give the title compound.
b. 3-(1-Methyl-4-phenyl-1H-imidazol-2-yl)-pyridine

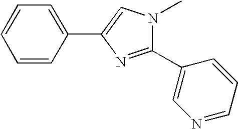

To a suspension of sodium hydride (922 mg, 60% mineral oil suspension, 23.0 mmol) in anhydrous DMF (20 mL) is added a solution of 3-(4-phenyl-1H-imidazol-2-yl)-pyridine (4.25 g, 19.2 mmol) in DMF (15 mL) at room temperature. The resulting mixture is stirred at 70° C. for 1 hour. Upon cooling to 0° C., iodomethane (3.0 g, 21.2 mmol) is added dropwise. The mixture is stirred at room temperature for 1 hour and at 70° C. for an additional 1 hour. The reaction mixture is cooled to room temperature and poured into ice-water (100 mL), and extracted with dichloromethane (60 mL×3). The organic layers are washed with water (30 mL×4) and brine, dried over anhydrous sodium sulfate, and solvent removed. The residue is purified by flash chromatography to give the title compound.
c. 3-(5-Bromo-1-methyl-4-phenyl-1H-imidazol-2-yl)-pyridine

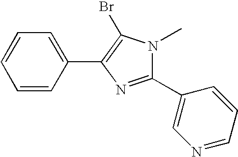

To a solution of 3-(1-methyl-4-phenyl-1H-imidazol-2-yl)-pyridine (3.53 g, 20 mmol) in acetonitrile (30 mL) at 0° C. is added NBS (3.56 g, 20 mmol) under nitrogen. The resulting mixture is stirred at room temperature for 2 hours. Ethyl acetate (80 mL) is added and the mixture is washed with water and brine, dried over sodium sulfate, and solvent removed. The crude is purified by flash chromatography to afford the title compound.
d. 3-Methyl-5-phenyl-2-pyridin-3-yl-3H-imidazole-4-carboxylic acid

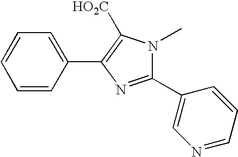

To a solution of 3-(5-bromo-1-methyl-4-phenyl-1H-imidazol-2-yl)pyridine (3.14 g, 10 mmol) in anhydrous tetrahydrofuran (50 mL) at −78° C. under nitrogen is added n-BuLi (2.5 M in hexane, 4.8 mL, 12mmol). The mixture is stirred at −78° C. for 60 minutes, and then quenched with carbon dioxide. The reaction mixture is transferred to a sealed flask, and gradually warmed to room temperature with stirring. After stirring at room temperature for an additional 30 minutes, the reaction mixture is quenched with water (40 mL), followed by evaporation of the THF. The residue is washed with ethyl acetate (30 mL×2). The aqueous layer is neutralized to pH=4˜5 with potassium carbonate, and extracted with dichloromethane. The organic layers are washed with water and brine, dried over Na₂SO₄, concentrated to give the title compound.
e. 3-Methyl-5-phenyl-2-pyridin-3-yl-3H-imidazole-4-carboxylic acid ((S)-1-phenyl-propyl)-amide

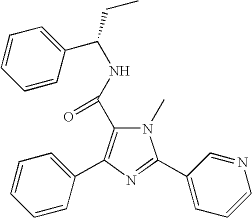

To a solution of 3-methyl-5-phenyl-2-pyridin-3-yl-3H-imidazole-4-carboxylic acid (80 mg, 0.29 mmol) and triethylamine (87 mg) in dichloromethane (5 mL) is added (s)-(−)-1-phenylpropylamine (46 mg, 0.35 mmol), followed by the addition of 2-chloro-1,3-dimethylimidazolidium chloride (63.7 mg, 0.37 mmol). The resulting mixture is stirred at room temperature for 3 hours. After the addition of water (10 mL), the mixture is extracted with dichloromethane. The organic layers are washed with water and brine, dried over sodium sulfate, and concentrated. The crude is then purified by PTLC to give the title compound. ¹H NMR (300 MHz, CDCl₃) δ 8.65 (1H, d), 8.17 (1H, d), 7.78 (1H, m), 7.58 (2H, m), 7.30-7.40 (3H, m), 7.29-7.23 (4H, m), 7.10-7.00 (2H, m), 5.96 (1H, d), 4.94 (1H, q), 4.28 (3H, s), 1.66 (2H, m), 0.76 (3H, t); MS (+VE) m/z 397 (M⁺+1).
B. 3-Methyl-5-phenyl-2-pyridin-2-yl-3H-imidazole-4-carboxylic acid ((S)-1-phenyl-propyl)-amide

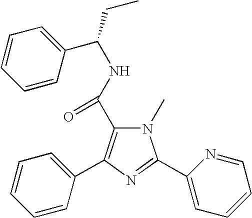

The title compound is prepared via a procedure analogous to that described above. ¹H NMR (300 MHz, CDCl₃) δ 8.90 (1H, d), 8.69 (1H, dd), 8.00 (1H, m), 7.58 -7.53 (2H, m), 7.45-7.23 (7H, m), 7.06-7.00 (2H, m), 6.02 (1H, d), 4.92 (1H, q), 3.94 (3H, s), 1.65 (2H, m), 0.76 (3H, t); MS (+VE) m/z 397 (M⁺+1).

### 2-6. GENERAL METHOD FOR PREPARATION OF 1-ALKYL-2,4-DIARYLIMIDAZOL-5-CARBOXAMIDES (SCHEME 5)

a. Ethyl 1-ethyl-4-(3-fluorophenyl)imidazol-5-carboxylate

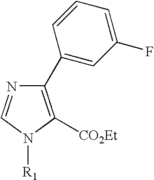

Ethyl glyoxalate (50% w/w in toluene, 8.40 g, 41.2 mmol) is added to a reaction vessel which is heated to 120° C. under N₂ for 1 hour. The reaction vessel is then placed in a 20° C. bath and THF (36 mL, anhydrous) and ethylamine (2 M in THF, 27.5 mL, 55 mmol) are added. After stirring 0.5 hours, [1-(3-fluorophenyl)-1-tosyl]methyl isocyanide (7.95 g, 27.5 mmol, synthesized essentially as described in Sisko et al. (1996) Tetrahedron Lett. 37:8113) and piperazine (3.05 g, 35.4 mmol) are added and the reaction is allowed to stir at room temperature overnight. The reaction is diluted with ether and transferred to a separatory funnel where it is washed with saturated NaHCO₃. The aqueous wash is reextracted with ether, and the combined organic extracts are washed with brine then dried over Na₂SO₄, filtered and concentrated in vacuo. The resulting oil is purified on a SiO₂ column eluted with 50% ethyl acetate/hexanes (v/v). The 0.4 rf (50% ethyl acetate/hexanes) component is collected and concentrated in vacuo.
b. Ethyl 2-bromo-1-ethyl-4-(3-fluorophenyl)imidazol-5-carboxylate

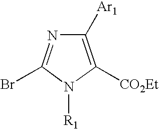

In a reaction vessel ethyl 1-ethyl-4-(3-fluorophenyl)imidazol-5-carboxylate (5.246 g, 20 mmol) is dissolved in carbon tetrachloride (180 mL, anhydrous) under N₂. NBS (5.34 g, 30 mmol) and AIBN (0.100 g, 0.61 mmol) are added and the reaction is heated to 60° C. After 4 hours, more AIBN (0.100 g, 0.61 mmol) is added, and after 6 hours more NBS (1.78 g, 10 mmol) and more AIBN (0.050 g, 0.30 mmol) are added. The reaction is kept at 60° C. overnight and is then transferred to a separatory funnel where it is washed with NaOH (1 M). The aqueous wash is reextracted with DCM, and the combined organic extracts are washed again with NaOH (1 M), then with brine, and then dried over Na₂SO₄, filtered and concentrated in vacuo. The resulting oil is purified on a SiO₂ column eluted with 10% ethyl acetate/hexanes (v/v). The 0.3 rf (10% ethyl acetate/hexanes) component is collected and concentrated in vacuo.
c. 2-Bromo-1-ethyl-4-(3-fluorophenyl)imidazol-5-carboxylic acid

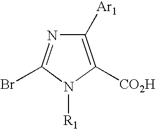

Ethyl 2-bromo-1-ethyl-4-(3-fluorophenyl)imidazol-5-carboxylate (1.78 g, 5.2 mmol) is dissolved in THF (5.2 mL), and NaOH (2.5 M, 2.6 mL, 6.5 mmol) is added. The reaction is heated to 50° C. for 36 hours. The resulting solution is brought to pH 4 with HBr (2 N) and extracted twice with ether. The combined organic extracts are washed with brine, dried over Na₂SO₄, filtered and concentrated in vacuo producing a tan powder
d. General methodfor preparation of 1-ethyl-2,4-diarylimidazol-5-carboxamides

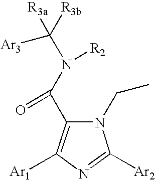

1-Alkyl-4-aryl-2-bromoimidazol-5-carboxylic acid (0.2 M in toluene with 5% DIEA; 0.100 mL; 0.020 mmol) is added to a reaction vessel followed by an amine (0.2 M in toluene; 0.110 mL; 0.022 mmol) and 2-chloro-1,3-dimethylimidazolinium chloride (0.2 M in acetonitrile; 0.110 mL; 0.022 mmol). The reaction vessel is allowed to stand at room temperature for 2 hours, and then is treated with potassium phosphate (tribasic, 1.0 M, 0.100 mL; 0.100 mmol), arylboronic acid (0.2 M in 1,4-dioxane; 0.150 mL, 0.030 mmol) and, under a nitrogen atmosphere, Pd(PPh₃)₄ (0.01 M in toluene, 0.050 mL; 0.0005 mmol). The reaction is heated to 80° C. under a nitrogen atmosphere for 14 hours, then treated with saturated NaHCO₃ (0.5 mL) and ethyl acetate (0.3 mL). The organic layer is purified via SCX SPE. By replacing ethyl iodide with other alkylating agents (e.g., methyl iodide), RI is varied to prepare additional compounds of Formula I.

### Example 3

### Additional Representative Diaryl Imidazole Derivatives

The following compounds are prepared by methods illustrated above. LC-MS data are given as HPLC retention times (in minutes) and M+1 (in amu). All compounds in Table 1 have an IC₅₀ of less than 4 micromolar in the assay of Example 5.

TABLE 1

| | | | Ret. | Calcd. | Obsd. |
|---|---|---|---|---|---|
| | Compound | Name | Time | Mass | M + H |
| 1 | 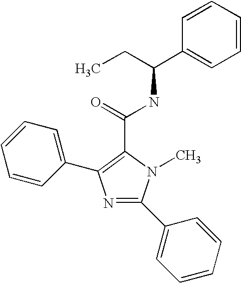 | 1-methyl-2,4-diphenyl-N-(1-phenyl- propyl)-1H-imidazole-5-car- boxamide | 1.11 | 395.2 | 396.2 |
| 2 | 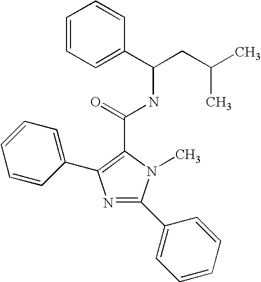 | 1-methyl-N-(3-methyl-1-phenyl- butyl)-2,4-diphenyl-1H-imi- dazole-5-carboxamide | 1.18 | 423.2 | 424.2 |
| 3 | 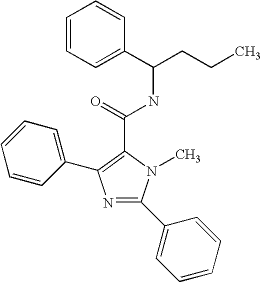 | 1-methyl-2,4-diphenyl-N-(1-phenyl- butyl)-1H-imidazole-5-car- boxamide | 1.16 | 409.2 | 410.3 |
| 4 | 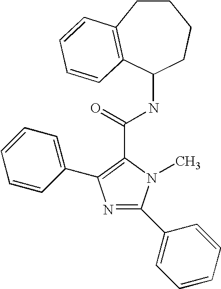 | 1-methyl-2,4-diphenyl-N-(6,7,8,9-tetra- hydro-5H-benzo[7]an- nulen-5-yl)-1H-imi- dazole-5-carboxamide | 1.15 | 421.2 | 422.3 |
| 5 | 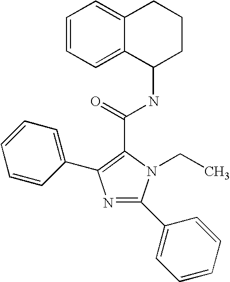 | 1-ethyl-2,4-diphenyl-N-(1,2,3,4-tetra- hydronaphthalen-1-yl)-1H-imi- dazole-5-carboxamide | 1.2 | 421.2 | 422.2 |
| 6 | 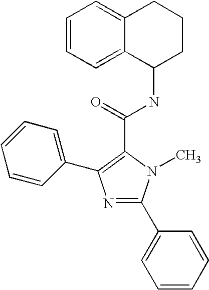 | 1-methyl-2,4-diphenyl-N-(1,2,3,4-tetra- hydronaphthalen-1-yl)-1H-imi- dazole-5-carboxamide | 1.19 | 407.2 | 408.2 |
| 7 | 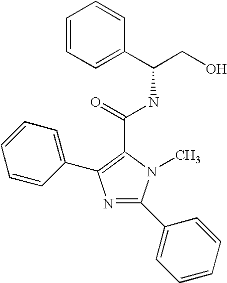 | N-(2-hydroxy-1-phenylethyl)-1-meth- yl-2,4-diphenyl-1H-imi- dazole-5-carboxamide | 1.1 | 397.2 | 398.2 |
| 8 | 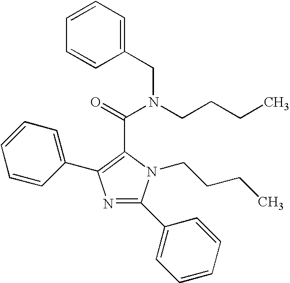 | N-benzyl-N,1-dibutyl-2,4-di- phenyl-1H-imidazole-5-car- boxamide | 1.24 | 465.3 | 466.3 |
| 9 | 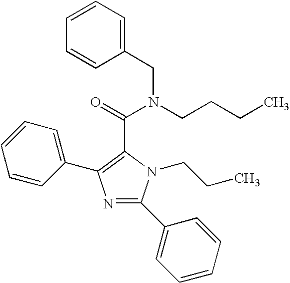 | N-benzyl-N-butyl-2,4-di- phenyl-1-propyl-1H-imi- dazole-5-carboxamide | 1.23 | 451.3 | 452.3 |
| 10 | 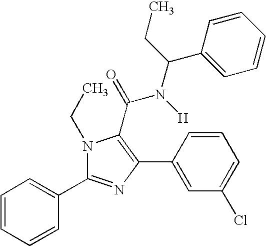 | 4-(3-chlorophenyl)-1-ethyl-2-phe- nyl-N-(1-phenylpropyl)-1H-imi- dazole-5-carboxamide | | | |
| 11 | 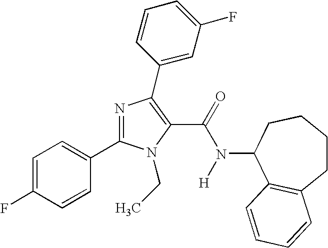 | 1-ethyl-4-(3-fluorophenyl)-2-(4-fluoro- phenyl)-N-(6,7,8,9-tetra- hydro-5H-ben- zo[7]annulen-5-yl)-1H-imi- dazole-5-carboxamide | 1.34 | 471.2 | 472.2 |
| 12 | 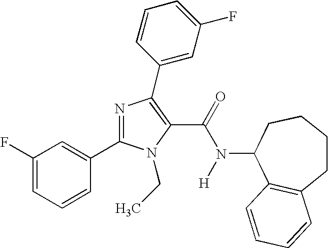 | 1-ethyl-2,4-bis(3-fluoro- phenyl)-N-(6,7,8,9-tetra- hydro-5H-ben- zo[7]annulen-5-yl)-1H-imi- dazole-5-carboxamide | 1.35 | 471.2 | 472.2 |
| 13 | 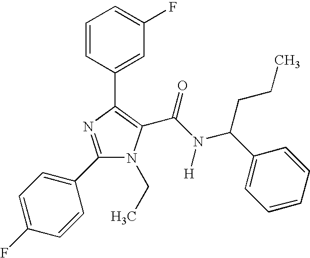 | 1-ethyl-4-(3-fluorophenyl)-2-(4-fluoro- phenyl)-N-(1-phenyl- butyl)-1H-imidazole-5-carboxamide | 1.33 | 459.2 | 460.2 |
| 14 | 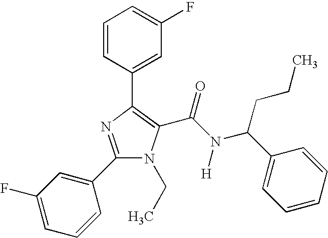 | 1-ethyl-2,4-bis(3-fluoro- phenyl)-N-(1-phenyl- butyl)-1H-imidazole-5-car- boxamide | 1.35 | 459.2 | 460.2 |
| 15 | 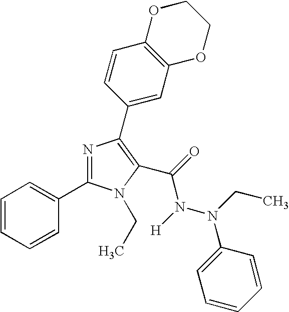 | 4-(2,3-dihydro-1,4-benzo- dioxin-6-yl)-N′,1-di- ethyl-N′,2-diphenyl-1H-imi- dazole-5-carbohydrazide | 1.21 | 468.2 | 469.2 |
| 16 | 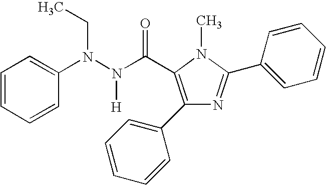 | N′-ethyl-1-methyl-N′,2,4-tri- phenyl-1H-imidazole-5-car- bohydrazide | 1.2 | 396.2 | 397.2 |
| 17 | 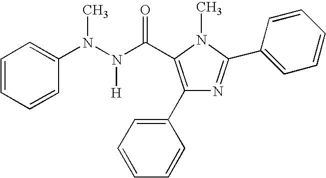 | N′,1-dimethyl-N′,2,4-tri- phenyl-1H-imidazole-5-car- bohydrazide | 1.19 | 382.2 | 383.2 |
| 18 | 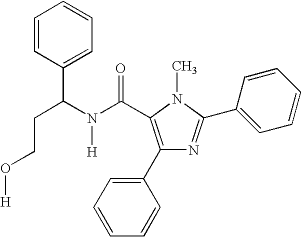 | N-(3-hydroxy-1-phenyl- propyl)-1-methyl-2,4-di- phenyl-1H-imidazole-5-car- boxamide | 1.17 | 411.2 | 412.2 |
| 19 | 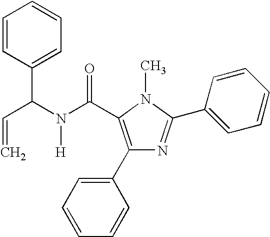 | 1-methyl-2,4-diphenyl-N-(1-phe- nylprop-2-en-1-yl)-1H-imi- dazole-5-carboxamide | 1.21 | 393.2 | 394.2 |
| 20 | 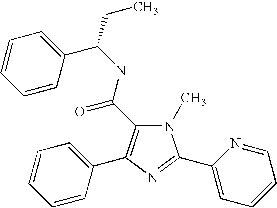 | 1-methyl-4-phenyl-N-(1-phenyl- propyl)-2-pyridin-2-yl-1H-imi- dazole-5-carboxamide | 1.27 | 396.2 | 397.2 |
| 21 | 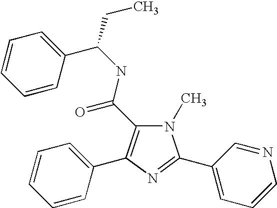 | 1-methyl-4-phenyl-N-(1-phenyl- propyl)-2-pyridin-3-yl-1H-imi- dazole-5-carboxamide | 1.25 | 396.2 | 397.2 |
| 22 | 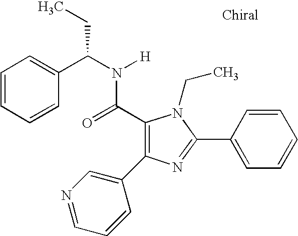 | 1-ethyl-2-phenyl-N-[(1S)-1-phenyl- propyl]-4-pyridin-3-yl-1H-imi- dazole-5-carboxamide | 1.22 | 410.2 | 411.2 |
| 23 | 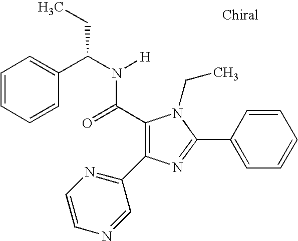 | 1-ethyl-2-phenyl-N-[(1S)-1-phenyl- propyl]-4-pyrazin-2-yl-1H-imi- dazole-5-carboxamide | 1.34 | 411.2 | 412.2 |
| 24 | 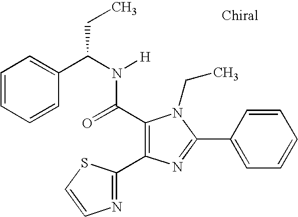 | 1-ethyl-2-phenyl-N-[(1S)-1-phenyl- propyl]-4-(1,3-thiazol-2-yl)-1H-imi- dazole-5-carboxamide | 1.44 | 416.2 | 417.1 |
| 25 | 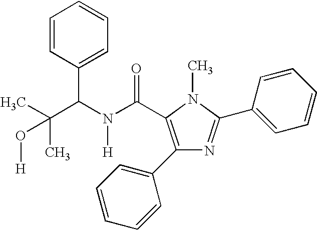 | N-(2-hydroxy-2-methyl-1-phenyl- propyl)-1-methyl-2,4-di- phenyl-1H-imidazole-5-car- boxamide | 1.2 | 425.2 | 426.2 |
| 26 | 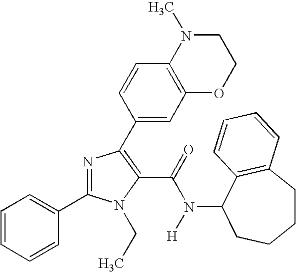 | 1-ethyl-4-(4-methyl-3,4-di- hydro-2H-1,4-benzoxazin-7-yl)-2-phe- nyl-N-(6,7,8,9-tetra- hydro-5H-benzo[7]an- nulen-5-yl)-1H-imi- dazole-5-carboxamide | 1.27 | 506.3 | 507.2 |
| 27 | 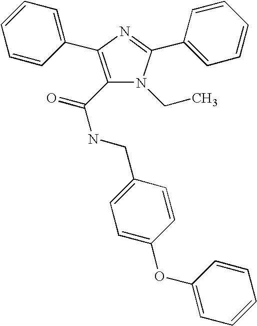 | 1-ethyl-N-(4-phenoxybenzyl)-2,4-di- phenyl-1H-imidazole-5-car- boxamide | 1.28 | 473.2 | 474.0 |
| 28 | 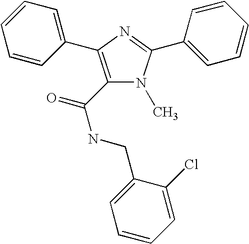 | N-(2-chlorobenzyl)-1-methyl-2,4-di- phenyl-1H-imidazole-5-car- boxamide | 1.2 | 401.1 | 401.9 |
| 29 | 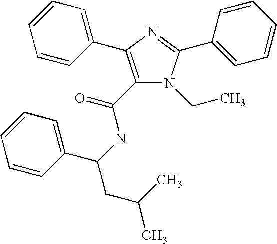 | 1-ethyl-N-(3-methyl-1-phenyl- butyl)-2,4-diphenyl-1H-imi- dazole-5-carboxamide | 1.27 | 437.2 | 438.0 |
| 30 | 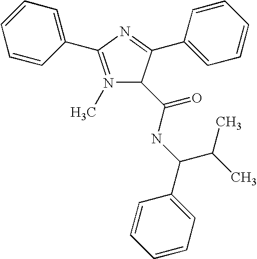 | 1-methyl-N-(2-methyl-1-phenyl- propyl)-2,4-diphenyl-1H-imi- dazole-5-carboxamide | 1.24 | 409.2 | 410.0 |
| 31 | 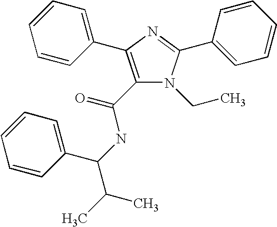 | 1-ethyl-N-(2-methyl-1-phenyl- propyl)-2,4-diphenyl-1H-imi- dazole-5-carboxamide | 1.25 | 423.2 | 424.0 |
| 32 | 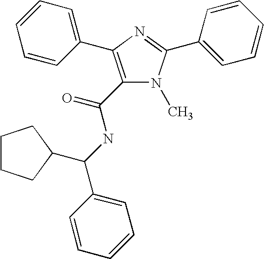 | N-[cyclo- pentyl(phenyl)methyl]-1-meth- yl-2,4-diphenyl-1H-imi- dazole-5-carboxamide | 1.27 | 435.2 | 436.0 |
| 33 | 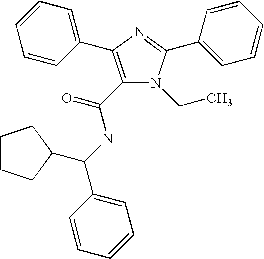 | N-[cyclo- pentyl(phenyl)methyl]-1-eth- yl-2,4-diphenyl-1H-imi- dazole-5-carboxamide | 1.28 | 449.2 | 450.0 |
| 34 | 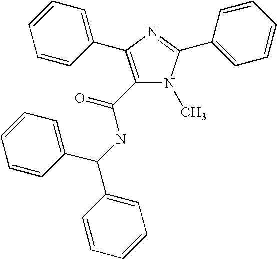 | N-(diphenylmethyl)-1-methyl-2,4-di- phenyl-1H-imidazole-5-car- boxamide | 1.26 | 443.2 | 444.0 |
| 35 | 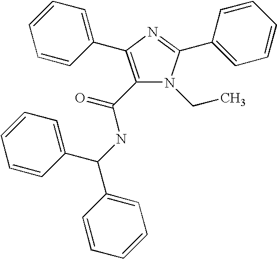 | N-(diphenylmethyl)-1-ethyl-2,4-di- phenyl-1H-imidazole-5-car- boxamide | 1.26 | 457.2 | 458.0 |
| 36 | 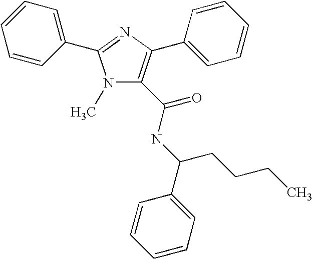 | 1-methyl-2,4-diphenyl-N-(1-phenyl- pentyl)-1H-imidazole-5-car- boxamide | 1.27 | 423.2 | 424.0 |
| 37 | 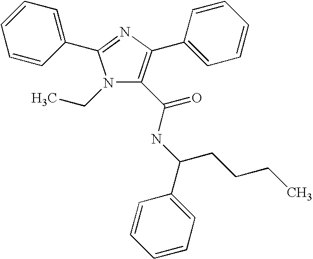 | 1-ethyl-2,4-diphenyl-N-(1-phenyl- pentyl)-1H-imidazole-5-car- boxamide | 1.28 | 437.2 | 438.0 |
| 38 | 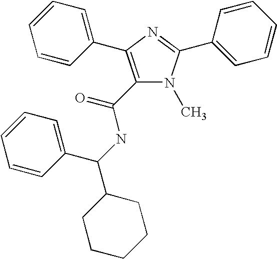 | N-[cyclo- hexyl(phenyl)methyl]-1-meth- yl-2,4-diphenyl-1H-imi- dazole-5-carboxamide | 1.29 | 449.2 | 450.0 |
| 39 | 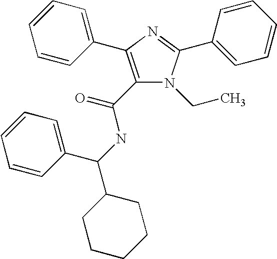 | N-[cyclo- hexyl(phenyl)methyl]-1-eth- yl-2,4-diphenyl-1H-imi- dazole-5-carboxamide | 1.31 | 463.3 | 464.0 |
| 40 | 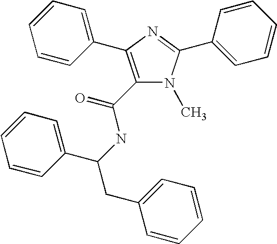 | N-(1,2-diphenylethyl)-1-meth- yl-2,4-diphenyl-1H-imi- dazole-5-carboxamide | 1.26 | 457.2 | 458.0 |
| 41 | 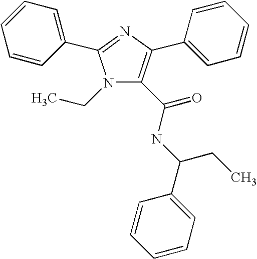 | 1-ethyl-2,4-diphenyl-N-(1-phenyl- propyl)-1H-imidazole-5-car- boxamide | 1.21 | 409.2 | 410.2 |
| 42 | 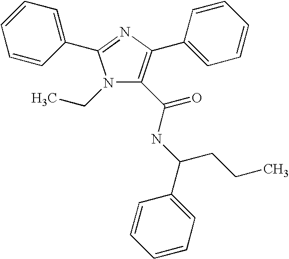 | 1-ethyl-2,4-diphenyl-N-(1-phenyl- butyl)-1H-imidazole-5-car- boxamide | 1.23 | 423.2 | 424.2 |
| 43 | 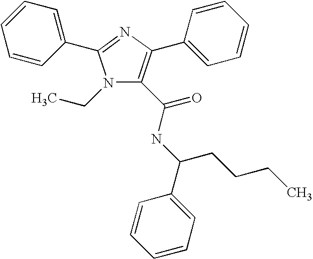 | 1-ethyl-2,4-diphenyl-N-(1-phenyl- pentyl)-1H-imidazole-5-carboxamide | 1.26 | 437.2 | 438.2 |
| 44 | 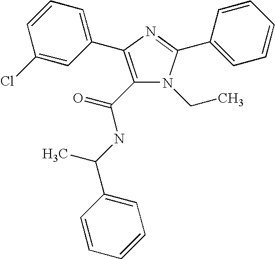 | 4-(3-chlorophenyl)-1-ethyl-2-phe- nyl-N-(1-phenylethyl)-1H-imi- dazole-5-carboxamide | 1.26 | 429.2 | 430.1 |
| 45 | 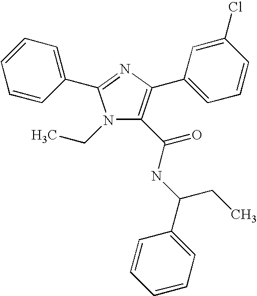 | 4-(3-chlorophenyl)-1-ethyl-2-phe- nyl-N-(phenylpropyl)-1H-imi- dazole-5-carboxamide | 1.27 | 443.2 | 444.1 |
| 46 | 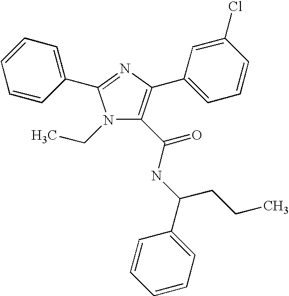 | 4-(3-chlorophenyl)-1-ethyl-2-phe- nyl-N-(1-phenylbutyl)-1H-imi- dazole-5-carboxamide | 1.3 | 457.2 | 458.2 |
| 47 | 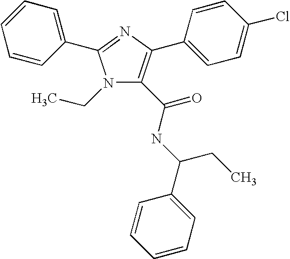 | 4-(4-chlorophenyl)-1-ethyl-2-phe- nyl-N-(1-phenylpropyl)-1H-imi- dazole-5-carboxamide | 1.26 | 443.2 | 444.1 |
| 48 | 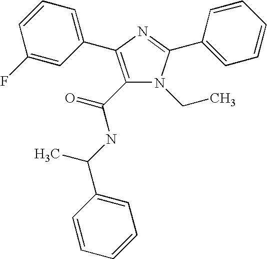 | 1-ethyl-4-(3-fluorophenyl)-2-phe- nyl-N-(1-phenylethyl)-1H-imi- dazole-5-carboxamide | 1.22 | 413.2 | 414.2 |
| 49 | 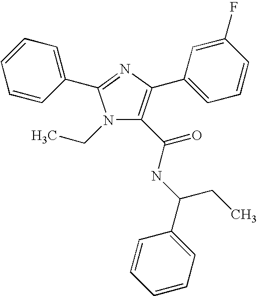 | 1-ethyl-4-(3-fluorophenyl)-2-phe- nyl-N-(1-phenylpropyl)-1H-imi- dazole-5-carboxamide | 1.24 | 427.2 | 428.2 |
| 50 | 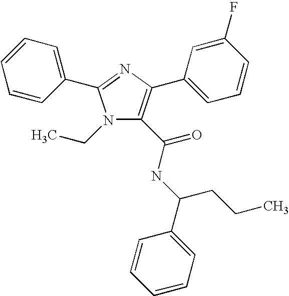 | 1-ethyl-4-(3-fluorophenyl)-2-phe- nyl-N-(1-phenylbutyl)-1H-imi- dazole-5-carboxamide | 1.27 | 441.2 | 442.2 |
| 51 | 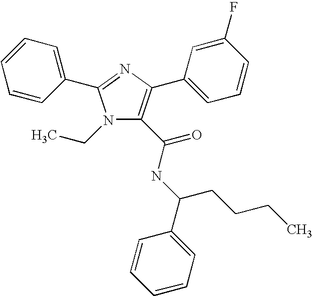 | 1-ethyl-4-(3-fluorophenyl)-2-phe- nyl-N-(1-phenylpentyl)-1H-imi- dazole-5-carboxamide | 1.29 | 455.2 | 456.2 |
| 52 | 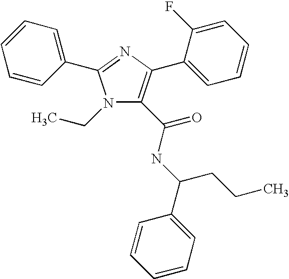 | 1-ethyl-4-(2-fluorophenyl)-2-phe- nyl-N-(1-phenylbutyl)-1H-imi- dazole-5-carboxamide | 1.25 | 441.2 | 442.2 |
| 53 | 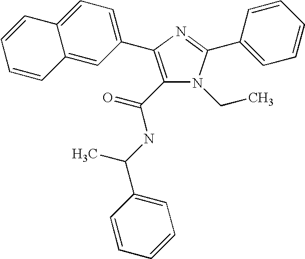 | 1-ethyl-4-(2-naphthyl)-2-phe- nyl-N-(1-phenylethyl)-1H-imi- dazole-5-carboxamide | | | |
| 54 | 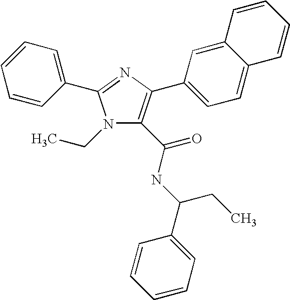 | 1-ethyl-4-(2-naphthyl)-2-phe- nyl-N-(1-phenylpropyl)-1H-imi- dazole-5-carboxamide | 1.25 | 459.2 | 460.2 |
| 55 | 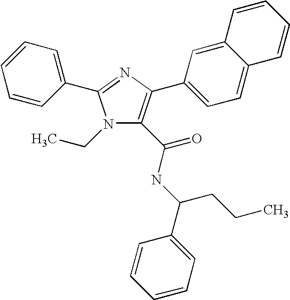 | 1-ethyl-4-(2-naphthyl)-2-phe- nyl-N-(1-phenylbutyl)-1H-imi- dazole-5-carboxamide | 1.28 | 473.2 | 474.2 |
| 56 | 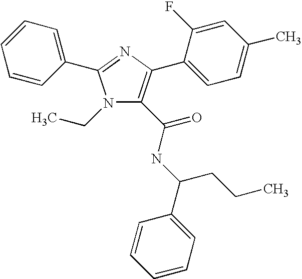 | 1-ethyl-4-(2-fluoro-4-meth- ylphenyl)-2-phenyl-N-(1-phenyl- butyl)-1H-imidazole-5-car- boxamide | 1.25 | 455.2 | 456.2 |
| 57 | 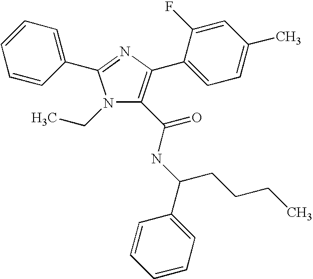 | 1-ethyl-4-(2-fluoro-4-meth- ylphenyl)-2-phenyl-N-(1-phenyl- pentyl)-1H-imidazole-5-car- boxamide | 1.28 | 469.3 | 471.2 |
| 58 | 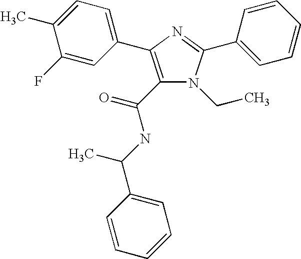 | 1-ethyl-4-(3-fluoro-4-meth- ylphenyl)-2-phenyl-N-(1-phenyl- ethyl)-1H-imidazole-5-car- boxamide | 1.23 | 427.2 | 428.2 |
| 59 | 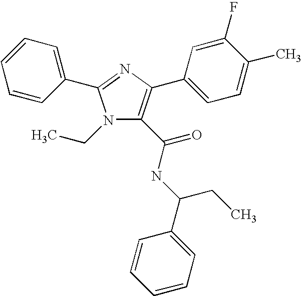 | 1-ethyl-4-(3-fluoro-4-meth- ylphenyl)-2-phenyl-N-(1-phenyl- propyl)-1H-imidazole-5-car- boxamide | 1.25 | 441.2 | 443.2 |
| 60 | 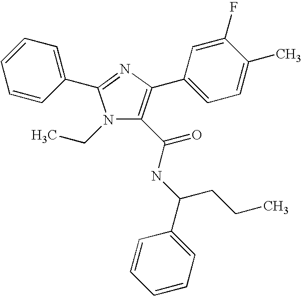 | 1-ethyl-4-(3-fluoro-4-meth- ylphenyl)-2-phenyl-N-(1-phenyl- butyl)-1H-imidazole-5-car- boxamide | 1.28 | 455.2 | 456.2 |
| 61 | 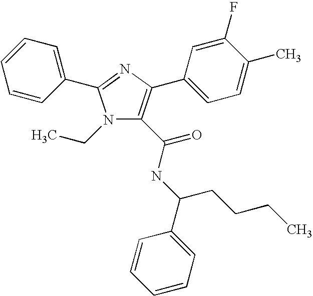 | 1-ethyl-4-(3-fluoro-4-meth- ylphenyl)-2-phenyl-N-(1-phenyl- pentyl)-1H-imidazole-5-car- boxamide | 1.3 | 469.3 | 470.2 |
| 62 | 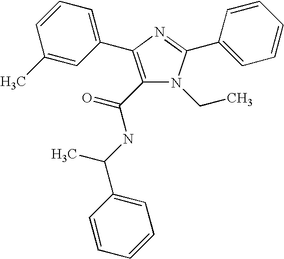 | 1-ethyl-4-(3-methylphenyl)-2-phe- nyl-N-(1-phenylethyl)-1H-imi- dazole-5-carboxamide | 1.21 | 409.2 | 410.2 |
| 63 | 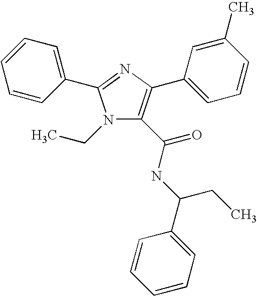 | 1-ethyl-4-(3-methylphenyl)-2-phe- nyl-N-(1-phenylpropyl)-1H-imi- dazole-5-carboxamide | 1.22 | 423.2 | 424.2 |
| 64 | 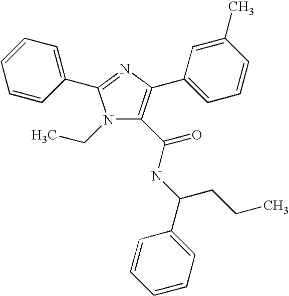 | 1-ethyl-4-(3-methylphenyl)-2-phe- nyl-N-(1-phenylbutyl)-1H-imi- dazole-5-carboxamide | 1.25 | 437.2 | 438.2 |
| 65 | 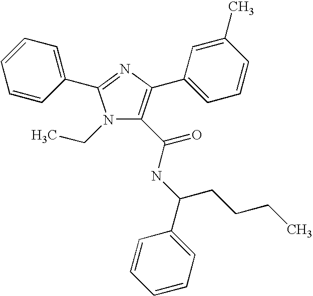 | 1-ethyl-4-(3-methylphenyl)-2-phe- nyl-N-(1-phenylpentyl)-1H-imi- dazole-5-carboxamide | 1.28 | 451.3 | 452.2 |
| 66 | 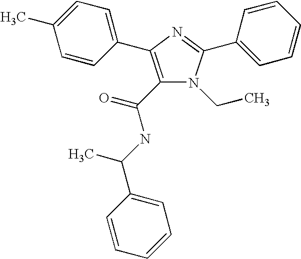 | 1-ethyl-4-(4-methylphenyl)-2-phe- nyl-N-(1-phenylethyl)-1H-imi- dazole-5-carboxamide | 1.2 | 409.2 | 410.2 |
| 67 | 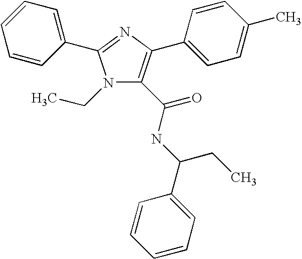 | 1-ethyl-4-(4-methylphenyl)-2-phe- nyl-N-(1-phenylpropyl)-1H-imi- dazole-5-carboxamide | 1.22 | 423.2 | 424.2 |
| 68 | 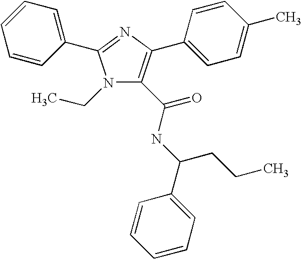 | 1-ethyl-4-(4-methylphenyl)-2-phe- nyl-N-(1-phenylbutyl)-1H-imi- dazole-5-carboxamide | 1.25 | 437.2 | 438.2 |
| 69 | 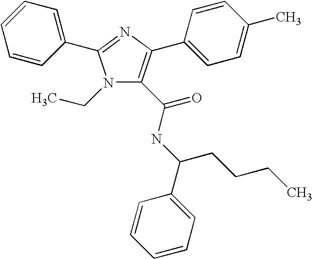 | 1-ethyl-4-(4-methylphenyl)-2-phe- nyl-N-(1-phenylpentyl)-1H-imi- dazole-5-carboxamide | 1.27 | 451.3 | 452.2 |
| 70 | 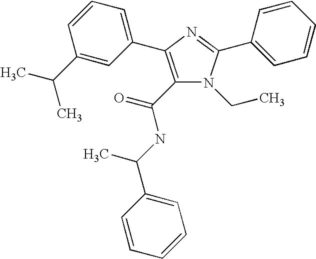 | 1-ethyl-4-(3-isopropylphenyl)-2-phe- nyl-N-(1-phenylethyl)-1H-imi- dazole-5-carboxamide | 1.24 | 437.2 | 438.2 |
| 71 | 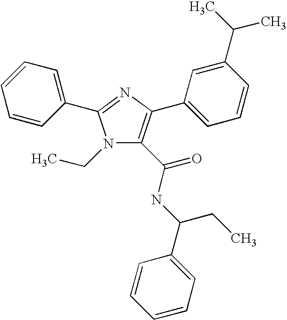 | 1-ethyl-4-(3-isopropylphenyl)-2-phe- nyl-N-(1-phenylpropyl)-1H-imi- dazole-5-carboxamide | 1.27 | 451.3 | 452.2 |
| 72 | 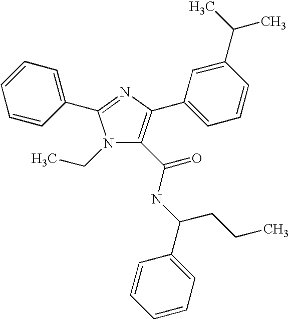 | 1-ethyl-4-(3-isopropylphenyl)-2-phe- nyl-N-(1-phenylbutyl)-1H-imi- dazole-5-carboxamide | 1.28 | 465.3 | 466.2 |
| 73 | 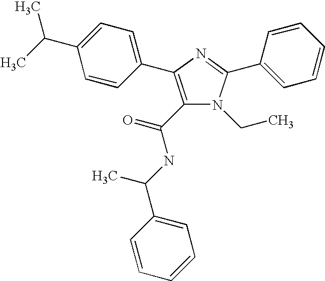 | 1-ethyl-4-(4-isopropylphenyl)-2-phe- nyl-N-(1-phenylethyl)-1H-imi- dazole-5-carboxamide | 1.25 | 437.2 | 438.2 |
| 74 | 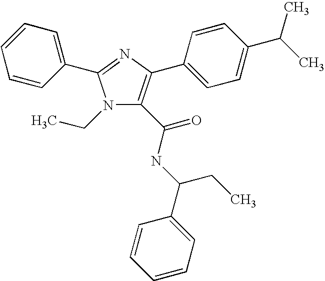 | 1-ethyl-4-(4-isopropylphenyl)-2-phe- nyl-N-(1-phenylpropyl)-1H-imi- dazole-5-carboxamide | 1.26 | 451.3 | 452.2 |
| 75 | 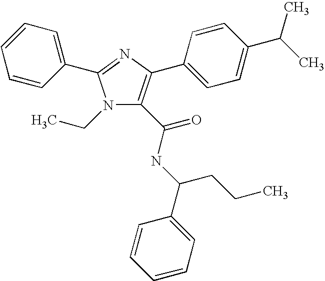 | 1-ethyl-4-(4-isopropylphenyl)-2-phe- nyl-N-(1-phenylbutyl)-1H-imi- dazole-5-carboxamide | 1.29 | 465.3 | 466.2 |
| 76 | 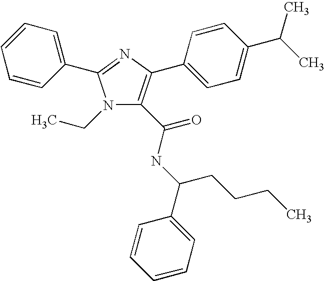 | 1-ethyl-4-(4-isopropylphenyl)-2-phe- nyl-N-(1-phenylpentyl)-1H-imi- dazole-5-carboxamide | 1.31 | 479.3 | 480.2 |
| 77 | 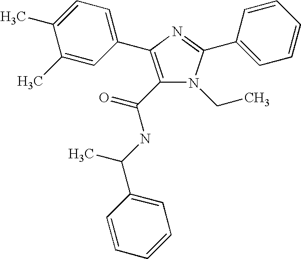 | 4-(3,4-dimethylphenyl)-1-eth- yl-2-phenyl-N-(1-phenyl- ethyl)-1H-imidazole-5-carboxamide | 1.22 | 423.2 | 424.2 |
| 78 | 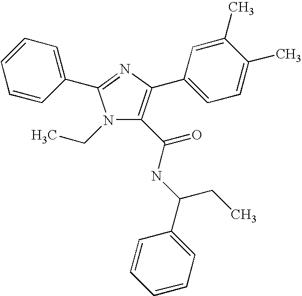 | 4-(3,4-dimethylphenyl)-1-eth- yl-2-phenyl-N-(1-phenyl- propyl)-1H-imidazole-5-car- boxamide | 1.23 | 437.2 | 438.2 |
| 79 | 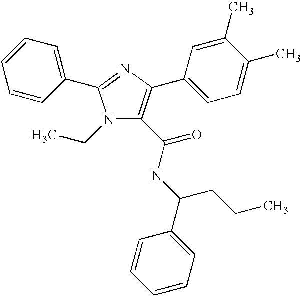 | 4-(3,4-dimethylphenyl)-1-eth- yl-2-phenyl-N-(1-phenyl- butyl)-1H-imidazole-5-car- boxamide | 1.26 | 451.3 | 425.2 |
| 80 | 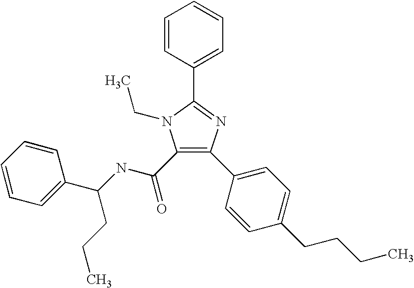 | 4-(4-butylphenyl)-1-ethyl-2-phe- nyl-N-(1-phenylbutyl)-1H-imi- dazole-5-carboxamide | 1.31 | 479.3 | 480.3 |
| 81 | 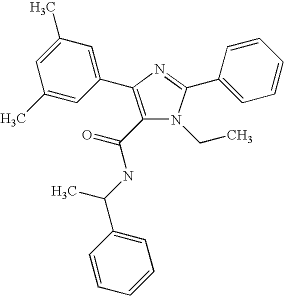 | 4-(3,5-dimethylphenyl)-1-eth- yl-2-phenyl-N-(1-phenyl- ethyl)-1H-imidazole-5-car- boxamide | 1.23 | 423.2 | 424.2 |
| 82 | 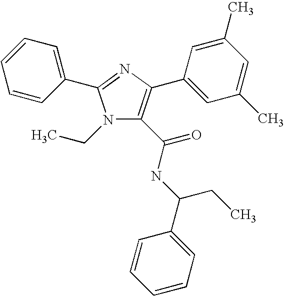 | 4-(3,5-dimethylphenyl)-1-eth- yl-2-phenyl-N-(1-phenyl- propyl)-1H-imidazole-5-car- boxamide | 1.23 | 437.2 | 438.2 |
| 83 | 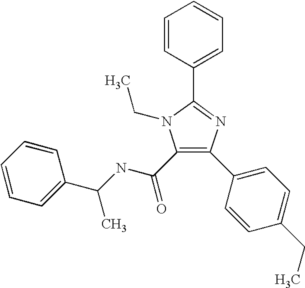 | 1-ethyl-4-(4-ethylphenyl)-2-phe- nyl-N-(1-phenylethyl)-1H-imi- dazole-5-carboxamide | 1.22 | 423.2 | 424.2 |
| 84 | 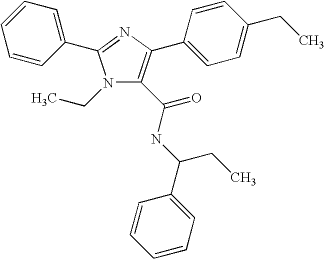 | 1-ethyl-4-(4-ethylphenyl)-2-phe- nyl-N-(1-phenylpropyl)-1H-imi- dazole-5-carboxamide | 1.24 | 437.2 | 438.2 |
| 85 | 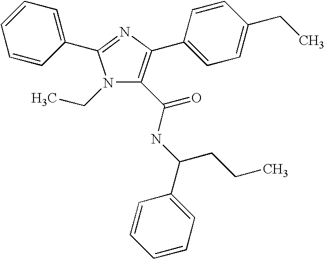 | 1-ethyl-4-(4-ethylphenyl)-2-phe- nyl-N-(1-phenylbutyl)-1H-imi- dazole-5-carboxamide | 1.27 | 451.3 | 452.2 |
| 86 | 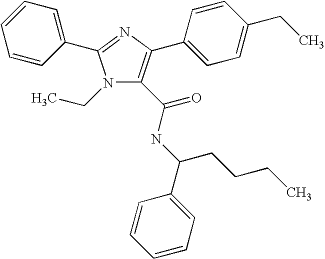 | 1-ethyl-4-(4-ethylphenyl)-2-phe- nyl-N-(1-phenylpentyl)-1H-imi- dazole-5-carboxamide | 1.29 | 465.3 | 466.2 |
| 87 | 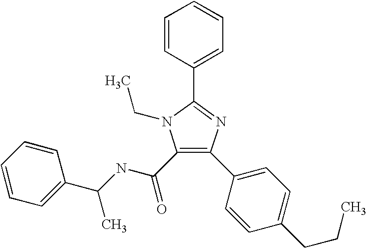 | 1-ethyl-2-phenyl-N-(1-phenyl- ethyl)-4-(4-propyl- phenyl)-1H-imidazole-5-car- boxamide | 1.25 | 437.2 | 438.2 |
| 88 | 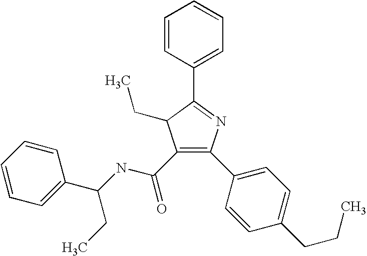 | 1-ethyl-2-phenyl-N-(1-phenyl- propyl)-4-(4-propyl- phenyl)-1H-imidazole-5-car- boxamide | 1.27 | 451.3 | 452.2 |
| 89 | 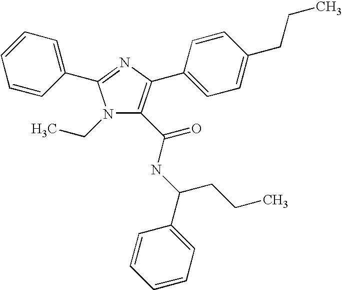 | 1-ethyl-2-phenyl-N-(1-phenyl- butyl)-4-(4-propyl- phenyl)-1H-imidazole-5-car- boxamide | 1.28 | 465.3 | 466.2 |
| 90 | 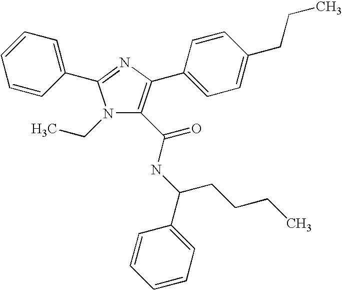 | 1-ethyl-2-phenyl-N-(1-phenyl- pentyl)-4-(4-propyl- phenyl)-1H-imidazole-5-car- boxamide | 1.31 | 479.3 | 480.2 |
| 91 | 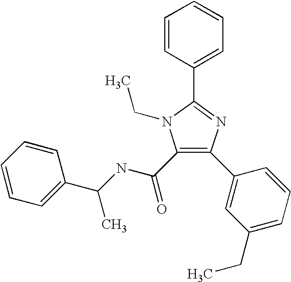 | 1-ethyl-4-(3-ethylphenyl)-2-phe- nyl-N-(1-phenylethyl)-1H-imi- dazole-5-carboxamide | 1.23 | 423.2 | 424.2 |
| 92 | 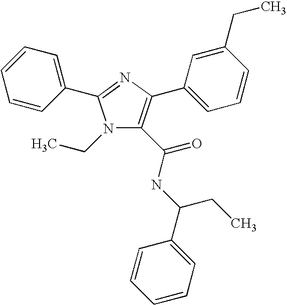 | 1-ethyl-4-(3-ethylphenyl)-2-phe- nyl-N-(1-phenylpropyl)-1H-imi- dazole-5-carboxamide | 1.24 | 437.2 | 438.2 |
| 93 | 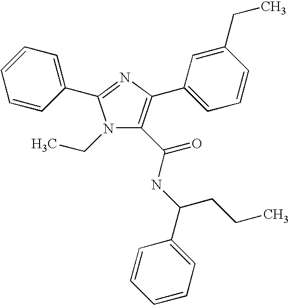 | 1-ethyl-4-(3-ethylphenyl)-2-phe- nyl-N-(1-phenylbutyl)-1H-imi- dazole-5-carboxamide | 1.27 | 451.3 | 452.2 |
| 94 | 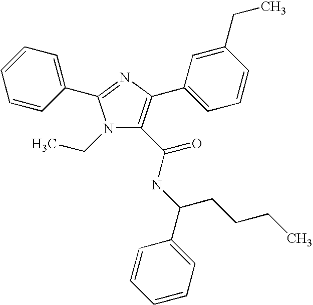 | 1-ethyl-4-(3-ethylphenyl)-2-phe- nyl-N-(1-phenylpentyl)-1H-imi- dazole-5-carboxamide | 1.28 | 465.3 | 466.2 |
| 95 | 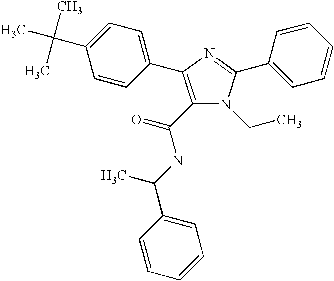 | 4-(4-tert-butylphenyl)-1-ethyl-2-phe- nyl-N-(1-phenylethyl)-1H-imi- dazole-5-carboxamide | 1.27 | 451.3 | 452.2 |
| 96 | 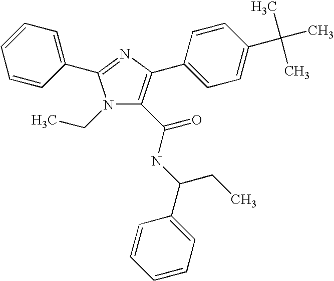 | 4-(4-tert-butylphenyl)-1-ethyl-2-phe- nyl-N-(1-phenylpropyl)-1H-imi- dazole-5-carboxamide | 1.28 | 465.3 | 466.2 |
| 97 | 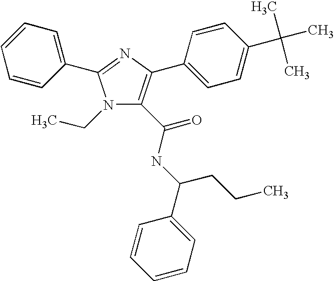 | 4-(4-tert-butylphenyl)-1-ethyl-2-phe- nyl-N-(1-phenylbutyl)-1H-imi- dazole-5-carboxamide | 1.3 | 479.3 | 480.3 |
| 98 | 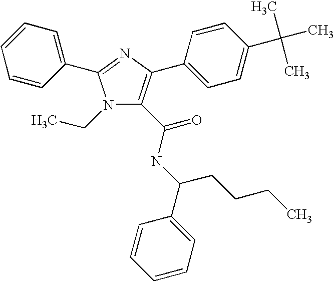 | 4-(4-tert-butylphenyl)-1-ethyl-2-phe- nyl-N-(1-phenylpentyl)-1H-imi- dazole-5-carboxamide | 1.33 | 493.3 | 494.3 |
| 99 | 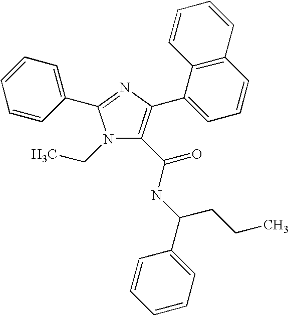 | 1-ethyl-4-(1-naphthyl)-2-phe- nyl-N-(1-phenylbutyl)-1H-imi- dazole-5-carboxamide | 1.29 | 473.2 | 474.3 |
| 100 | 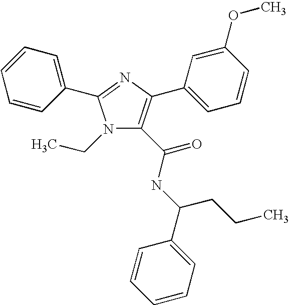 | 1-ethyl-4-(3-methoxyphenyl)-2-phe- nyl-N-(1-phenylbutyl)-1H-imi- dazole-5-carboxamide | 1.25 | 453.2 | 454.3 |
| 101 | 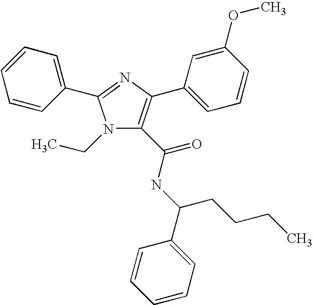 | 1-ethyl-4-(3-methoxyphenyl)-2-phe- nyl-N-(1-phenylpentyl)-1H-imi- dazole-5-carboxamide | 1.28 | 467.3 | 468.3 |
| 102 | 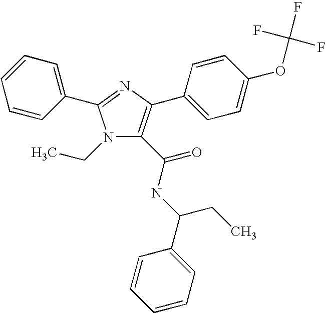 | 1-ethyl-2-phenyl-N-(1-phenyl- propyl)-4-[4-(tri- fluoromethoxy)phenyl]-1H-imi- dazole-5-carboxamide | 1.31 | 493.2 | 494.3 |
| 103 | 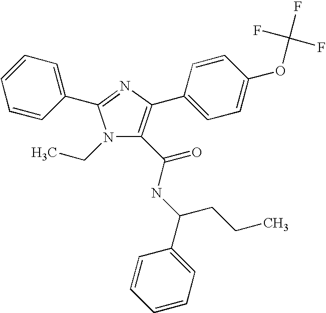 | 1-ethyl-2-phenyl-N-(1-phenyl- butyl)-4-[4-(tri- fluoromethoxy)phenyl]-1H-imi- dazole-5-carboxamide | 1.33 | 507.2 | 508.3 |
| 104 | 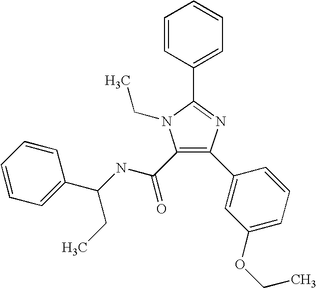 | 4-(3-ethoxyphenyl)-1-ethy-2-phe- nyl-N-(1-phenylpropyl)-1H-imi- dazole-5-carboxamide | 1.25 | 453.2 | 454.3 |
| 105 | 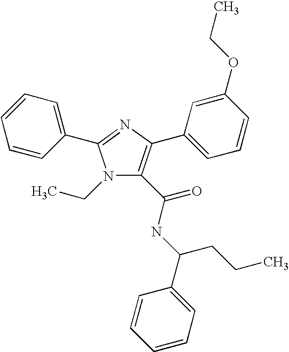 | 4-(3-ethoxyphenyl)-1-ethyl-2-phe- nyl-N-(1-phenylbutyl)-1H-imi- dazole-5-carboxamide | 1.27 | 467.3 | 468.3 |
| 106 | 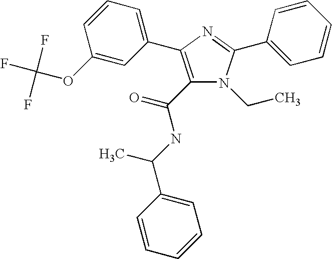 | 1-ethyl-2-phenyl-N-(1-phenyl- ethyl)-4-[3-(tri- fluoromethoxy)phenyl]-1H-imi- dazole-5-carboxamide | 1.29 | 479.2 | 481.3 |
| 107 | 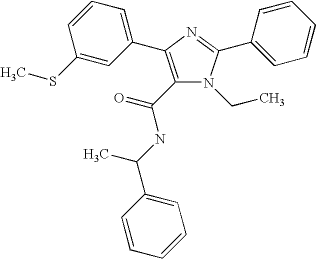 | 1-ethyl-4-[3-(meth- ylthio)phenyl]-2-phenyl-N-(1-phenyl- ethyl)-1H-imidazole-5-car- boxamide | 1.24 | 441.2 | 442.2 |
| 108 | 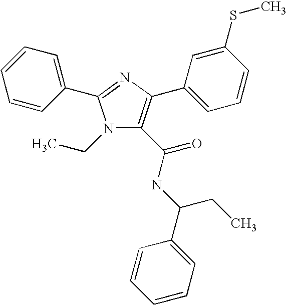 | 1-ethyl-4-[3-(meth- ylthio)phenyl]-2-phenyl-N-(1-phenyl- propyl)-1H-imi- dazole-5-carboxamide | 1.25 | 455.2 | 456.3 |
| 109 | 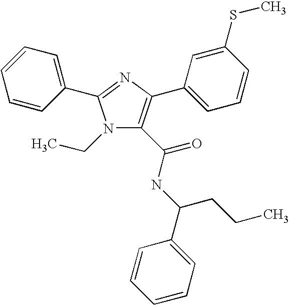 | 1-ethyl-4-[3-(methyl- thio)phenyl]-2-phenyl-N-(1-phenyl- butyl)-1H-imi- dazole-5-carboxamide | 1.28 | 469.2 | 470.2 |
| 110 | 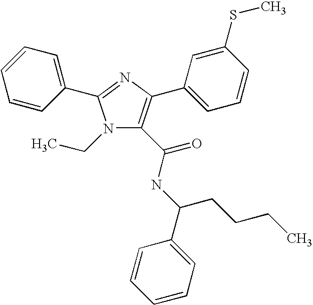 | 1-ethyl-4-[3-(methyl- thio)phenyl]-2-phenyl-N-(1-phenyl- pentyl)-1H-imi- dazole-5-carboxamide | 1.31 | 483.2 | 483.8 |
| 111 | 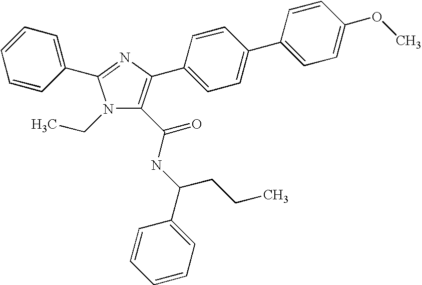 | 1-ethyl-4-(4′-meth- oxybiphenyl-4-yl)-2-phe- nyl-N-(1-phenylbutyl)-1H-imi- dazole-5-carboxamide | 1.31 | 529.3 | 530.4 |
| 112 | 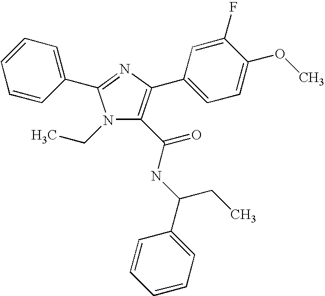 | 1-ethyl-4-(3-fluoro-4-meth- oxyphenyl)-2-phenyl-N-(1-phenyl- propyl)-1H-imi- dazole-5-carboxamide | 1.23 | 457.2 | 458.3 |
| 113 | 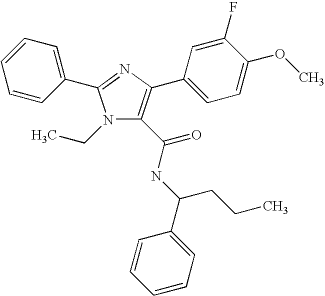 | 1-ethyl-4-(3-fluoro-4-meth- oxyphenyl)-2-phenyl-N-(1-phenyl- butyl)-1H-imi- dazole-5-carboxamide | 1.25 | 471.2 | 472.3 |
| 114 | 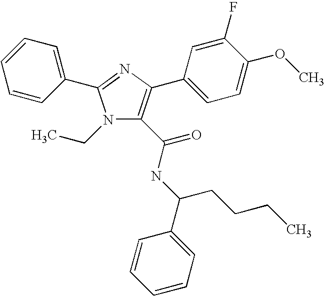 | 1-ethyl-4-(3-fluoro-4-meth- oxyphenyl)-2-phenyl-N-(1-phenyl- pentyl)-1H-imi- dazole-5-carboxamide | 1.27 | 485.2 | 486.3 |
| 115 | 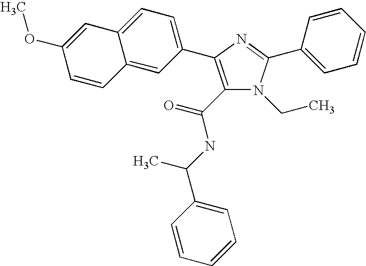 | 1-ethyl-4-(6-methoxy-2-naph- thyl)-2-phenyl-N-(1-phenyl- ethyl)-1H-imidazole-5-car- boxamide | 1.24 | 475.2 | 476.3 |
| 116 | 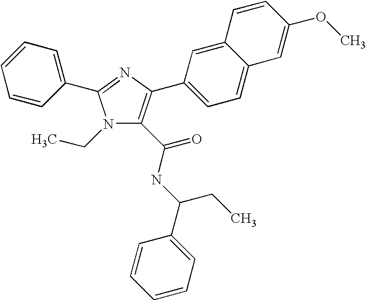 | 1-ethyl-4-(6-methoxy-2-naph- thyl)-2-phenyl-N-(1-phenyl- propyl)-1H-imidazole-5-car- boxamide | 1.25 | 489.2 | 490.3 |
| 117 | 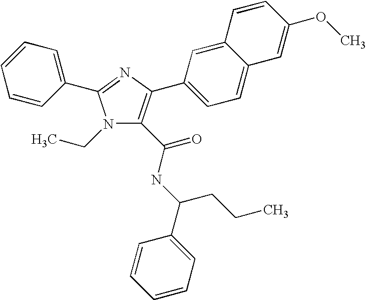 | 1-ethyl-4-(6-methoxy-2-naph- thyl)-2-phenyl-N-(1-phenyl- butyl)-1H-imidazole-5-car- boxamide | 1.28 | 503.3 | 504.3 |
| 118 | 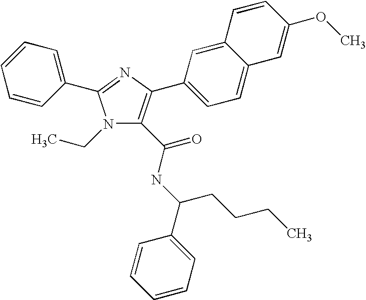 | 1-ethyl-4-(6-methoxy-2-naph- thyl)-2-phenyl-N-(1-phenyl- pentyl)-1H-imidazole-5-car- boxamide | 1.3 | 517.3 | 518.3 |
| 119 | 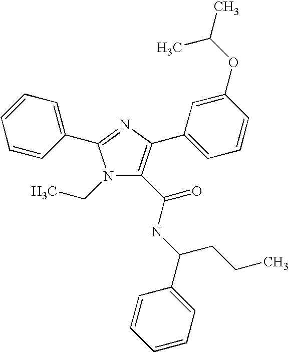 | 1-ethyl-4-(3-iso- propoxyphenyl)-2-phenyl-N-(1-phenyl- butyl)-1H-imi- dazole-5-carboxamide | 1.29 | 481.3 | 482.3 |
| 120 | 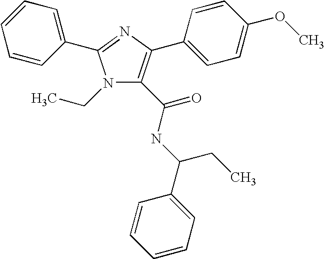 | 1-ethyl-4-(4-methoxyphenyl)-2-phe- nyl-N-(1-phenylpropyl)-1H-imi- dazole-5-carboxamide | 1.21 | 439.2 | 440.3 |
| 121 | 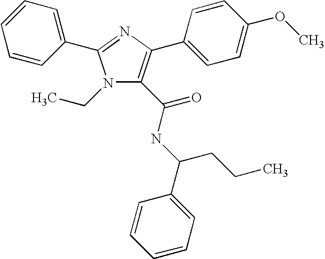 | 1-ethyl-4-(4-methoxyphenyl)-2-phe- nyl-N-(1-phenylbutyl)-1H-imi- dazole-5-carboxamide | 1.24 | 453.2 | 454.3 |
| 122 | 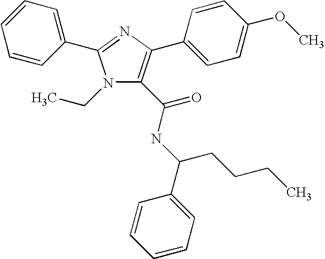 | 1-ethyl-4-(4-methoxyphenyl)-2-phe- nyl-N-(1-phenylpentyl)-1H-imi- dazole-5-carboxamide | | | |
| 123 | 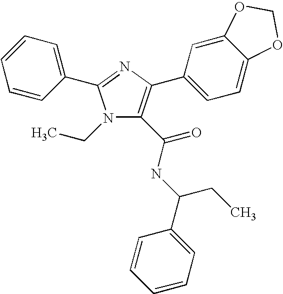 | 4-(1,3-benzodioxol-5-yl)-1-eth- yl-2-phenyl-N-(1-phenyl- propyl)-1H-imidazole-5-car- boxamide | 1.22 | 453.2 | 454.3 |
| 124 | 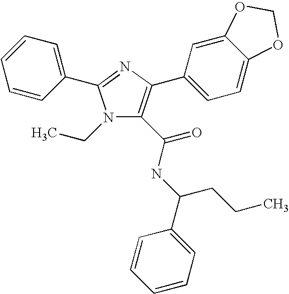 | 4-(1,3-benzodioxol-5-yl)-1-eth- yl-2-phenyl-N-(1-phenyl- butyl)-1H-imidazole-5-car- boxamide | 1.24 | 467.2 | 468.3 |
| 125 | 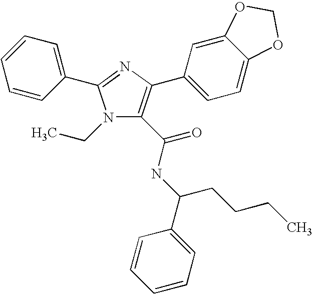 | 4-(1,3-benzodioxol-5-yl)-1-eth- yl-2-phenyl-N-(1-phenyl- pentyl)-1H-imidazole-5-car- boxamide | 1.26 | 481.2 | 482.3 |
| 126 | 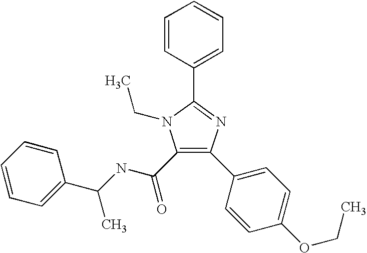 | 4-(4-ethoxyphenyl)-1-ethyl-2-phe- nyl-N-(1-phenylethyl)-1H-imi- dazole-5-carboxamide | 1.22 | 439.2 | 440.3 |
| 127 | 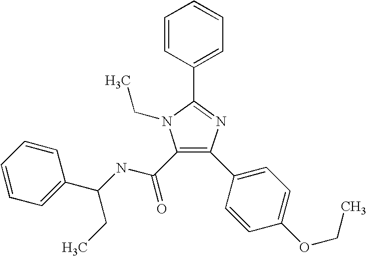 | 4-(4-ethoxyphenyl)-1-ethyl-2-phe- nyl-N-(1-phenylpropyl)-1H-imi- dazole-5-carboxamide | 1.23 | 453.2 | 454.3 |
| 128 | 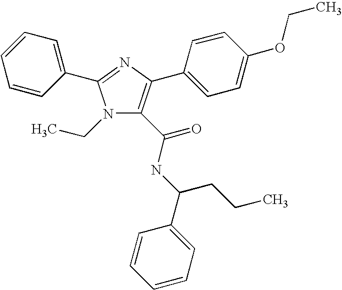 | 4-(4-ethoxyphenyl)-1-ethyl-2-phe- nyl-N-(1-phenylbutyl)-1H-imi- dazole-5-carboxamide | 1.27 | 467.3 | 468.3 |
| 129 | 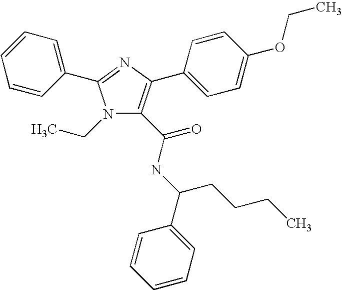 | 4-(4-ethoxyphenyl)-1-ethyl-2-phe- nyl-N-(1-phenylpentyl)-1H-imi- dazole-5-carboxamide | 1.29 | 481.3 | 482.3 |
| 130 | 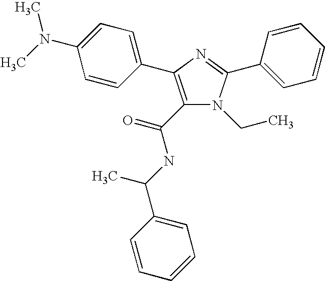 | 4-[4-(dimethylamino)phenyl]-1-eth- yl-2-phenyl-N-(1-phenyl- ethyl)-1H-imidazole-5-car- boxamide | 1.2 | 438.2 | 439.3 |
| 131 | 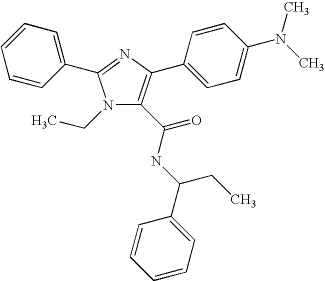 | 4-[4-(dimethylamino)phenyl]-1-eth- yl-2-phenyl-N-(1-phenyl- propyl)-1H-imidazole-5-car- boxamide | 1.22 | 452.3 | 453.3 |
| 132 | 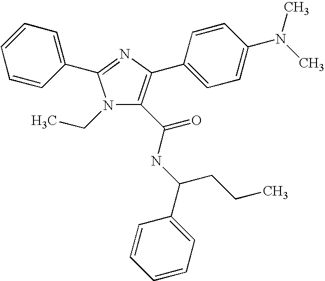 | 4-[4-(dimethylamino)phenyl]-1-eth- yl-2-phenyl-N-(1-phenyl- butyl)-1H-imidazole-5-car- boxamide | 1.24 | 466.3 | 467.3 |
| 133 | 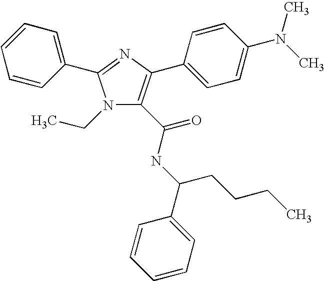 | 4-[4-(dimethylamino)phenyl]-1-eth- yl-2-phenyl-N-(1-phenyl- pentyl)-1H-imidazole-5-car- boxamide | 1.27 | 480.3 | 481.4 |
| 134 | 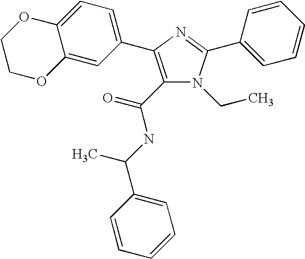 | 4-(2,3-dihydro-1,4-benzo- dioxin-6-yl)-1-ethyl-2-phe- nyl-N-(1-phenylethyl)-1H-imi- dazole-5-carboxamide | 1.19 | 453.2 | 454.3 |
| 135 | 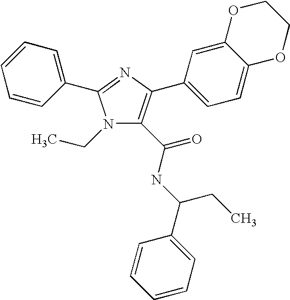 | 4-(2,3-dihydro-1,4-benzo- dioxin-6-yl)-1-ethyl-2-phe- nyl-N-(1-phenylpropyl)-1H-imi- dazole-5-carboxamide | 1.21 | 467.2 | 468.3 |
| 136 | 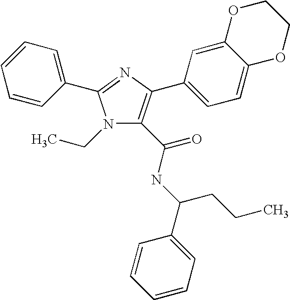 | 4-(2,3-dihydro-1,4-benzo- dioxin-6-yl)-1-ethyl-2-phe- nyl-N-(1-phenylbutyl)-1H-imi- dazol-5-carboxamide | 1.23 | 481.2 | 482.3 |
| 137 | 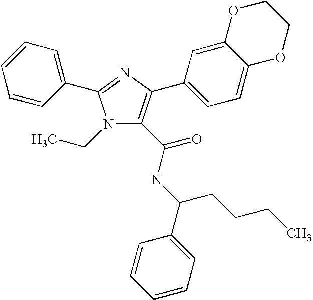 | 4-(2,3-dihydro-1,4-benzo- dioxin-6-yl)-1-ethyl-2-phe- nyl-N-(1-phenylpentyl)-1H-imi- dazole-5-carboxamide | 1.26 | 495.3 | 496.3 |
| 138 | 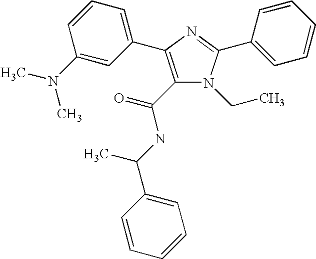 | 4-[3-(dimethylamino)phenyl]-1-eth- yl-2-phenyl-N-(1-phenyl- ethyl)-1H-imidazole-5-car- boxamide | 1.2 | 438.2 | 439.3 |
| 139 | 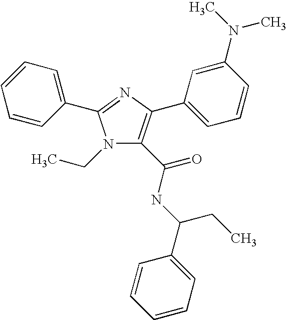 | 4-[3-(dimethylamino)phenyl]-1-eth- yl-2-phenyl-N-(1-phenyl- propyl)-1H-imidazole-5-car- boxamide | 1.22 | 452.3 | 453.3 |
| 140 | 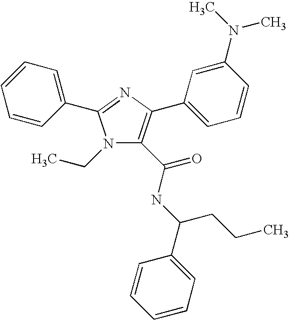 | 4-[3-(dimethylamino)phenyl]-1-eth- yl-2-phenyl-N-(1-phenyl- butyl)-1H-imidazole-5-car- boxamide | 1.25 | 466.3 | 467.3 |
| 141 | 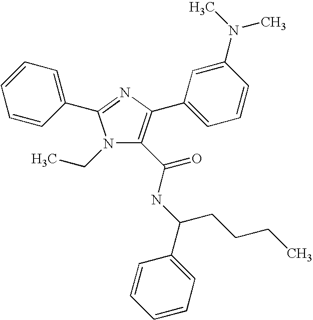 | 4-[3-(dimethylamino)phenyl]-1-eth- yl-2-phenyl-N-(1-phenyl- pentyl)-1H-imidazole-5-car- boxamide | 1.27 | 480.3 | 481.4 |
| 142 | 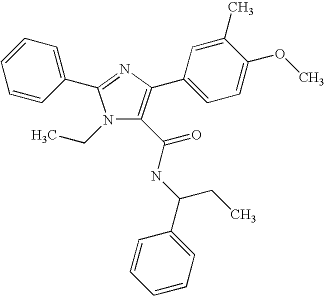 | 1-ethyl-4-(4-methoxy-3-meth- ylphenyl)-2-phenyl-N-(1-phenyl- propyl)-1H-imidazole-5-car- boxamide | 1.24 | 453.2 | 454.3 |
| 143 | 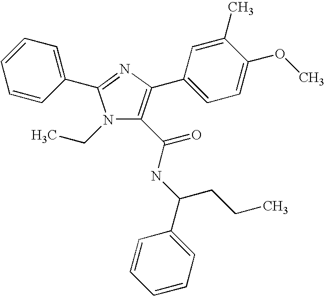 | 1-ethyl-4-(4-methoxy-3-meth- ylphenyl)-2-phenyl-N-(1-phenyl- butyl)-1H-imidazole-5-car- boxamide | 1.26 | 467.3 | 468.3 |
| 144 | 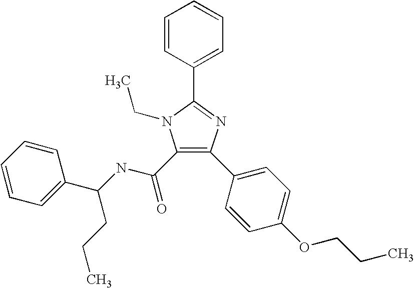 | 1-ethyl-2-phenyl-N-(1-phenyl- butyl)-4-(4-pro- poxyphenyl)-1H-imi- dazole-5-carboxamide | 1.29 | 481.3 | 482.3 |
| 145 | 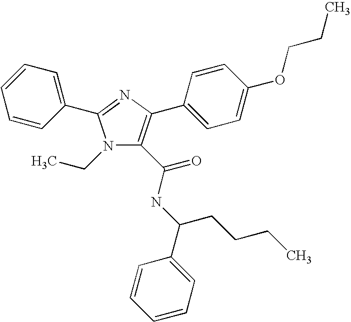 | 1-ethyl-2-phenyl-N-(1-phenyl- pentyl)-4-(4-pro- poxyphenyl)-1H-imi- dazole-5-carboxamide | 1.32 | 495.3 | 497.4 |
| 146 | 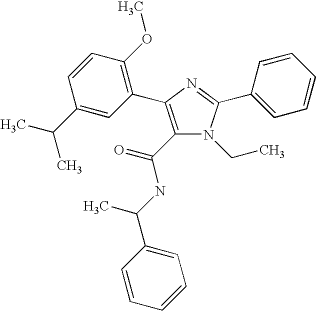 | 1-ethyl-4-(5-isopropyl-2-meth- oxyphenyl)-2-phenyl-N-(1-phenyl- ethyl)-1H-imi- dazole-5-carboxamide | | | |
| 147 | 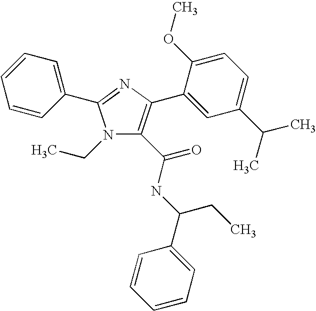 | 1-ethyl-4-(5-isopropyl-2-meth- oxyphenyl)-2-phenyl-N-(1-phenyl- propyl)-1H-imi- dazole-5-carboxamide | | | |
| 148 | 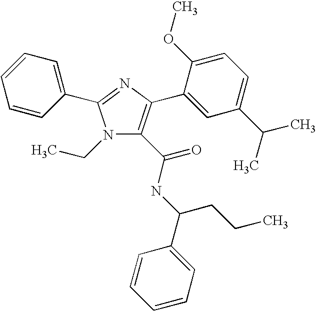 | 1-ethyl-4-(5-isopropyl-2-meth- oxyphenyl)-2-phenyl-N-(1-phenyl- butyl)-1H-imi- dazole-5-carboxamide | 1.3 | 495.3 | 496.4 |
| 149 | 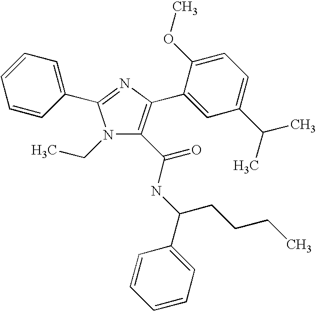 | 1-ethyl-4-(5-isopropyl-2-meth- oxyphenyl)-2-phenyl-N-(1-phenyl- pentyl)-1H-imi- dazole-5-carboxamide | 1.32 | 509.3 | 510.4 |
| 150 | 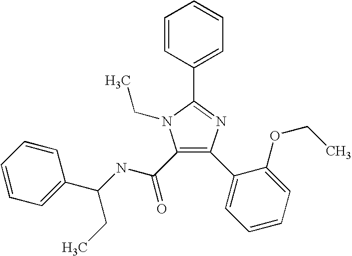 | 4-(2-ethoxyphenyl)-1-ethyl-2-phe- nyl-N-(1-phenylpropyl)-1H-imi- dazole-5-carboxamide | 1.22 | 453.2 | 455.3 |
| 151 | 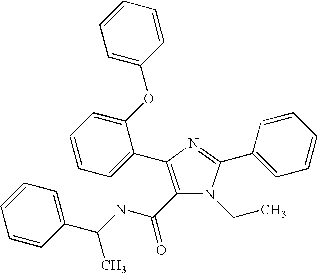 | 1-ethyl-4-(2-phenoxyphenyl)-2-phe- nyl-N-(1-phenylethyl)-1H-imi- dazole-5-carboxamide | 1.26 | 487.2 | 489.3 |
| 152 | 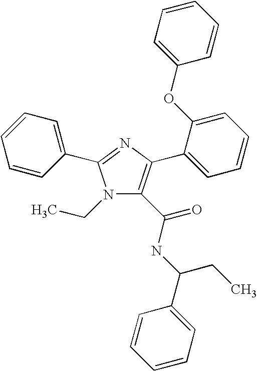 | 1-ethyl-4-(2-phenoxyphenyl)-2-phe- nyl-N-(1-phenylpropyl)-1H-imi- dazole-5-carboxamide | 1.28 | 501.2 | 502.2 |
| 153 | 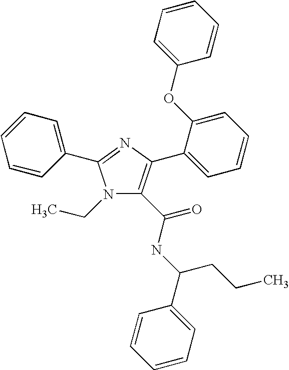 | 1-ethyl-4-(2-phenoxyphenyl)-2-phe- nyl-N-(1-phenylbutyl)-1H-imi- dazole-5-carboxamide | 1.3 | 515.3 | 517.3 |
| 154 | 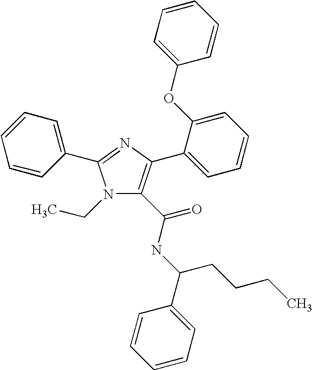 | 1-ethyl-4-(2-phenoxyphenyl)-2-phe- nyl-N-(1-phenylpentyl)-1H-imi- dazole-5-carboxamide | 1.32 | 529.3 | 531.4 |
| 155 | 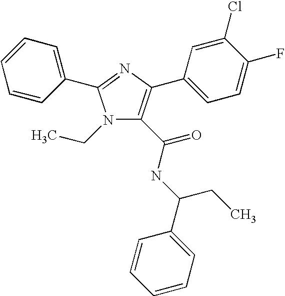 | 4-(3-chloro-4-fluorophenyl)-1-eth- yl-2-phenyl-N-(1-phenyl- propyl)-1H-imidazole-5-car- boxamide | 1.3 | 461.2 | 462.2 |
| 156 | 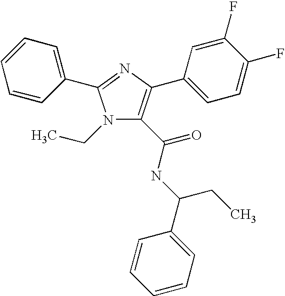 | 4-(3,4-difluorophenyl)-1-eth- yl-2-phenyl-N-(1-phenyl- propyl)-1H-imidazole-5-car- boxamide | 1.28 | 445.2 | 446.3 |
| 157 | 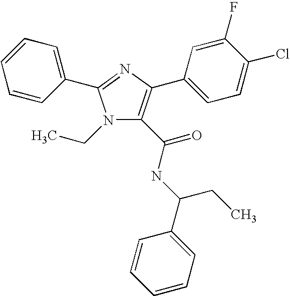 | 4-(4-chloro-3-fluorophenyl)-1-eth- yl-2-phenyl-N-(1-phenyl- propyl)-1H-imidazole-5-car- boxamide | 1.31 | 461.2 | 462.2 |
| 158 | 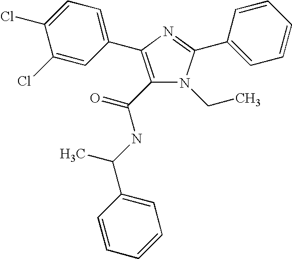 | 4-(3,4-dichlorophenyl)-1-eth- yl-2-phenyl-N-(1-phenyl- ethyl)-1H-imidazole-5-car- boxamide | 1.32 | 463.1 | 464.2 |
| 159 | 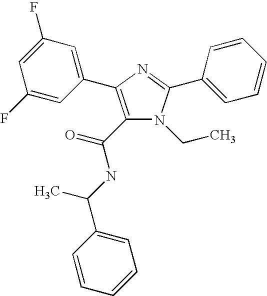 | 4-(3,5-difluorophenyl)-1-eth- yl-2-phenyl-N-(1-phenyl- ethyl)-1H-imidazole-5-car- boxamide | 1.27 | 431.2 | 432.2 |
| 160 | 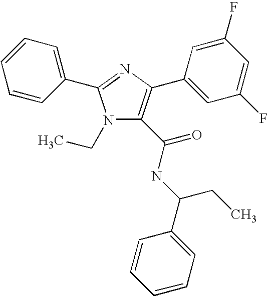 | 4-(3,5-difluorophenyl)-1-eth- yl-2-phenyl-N-(1-phenyl- propyl)-1H-imidazole-5-car- boxamide | 1.3 | 445.2 | 446.3 |
| 161 | 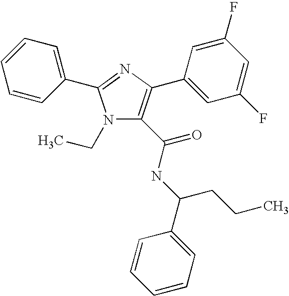 | 4-(3,5-difluorophenyl)-1-eth- yl-2-phenyl-N-(1-phenyl- butyl)-1H-imidazole-5-car- boxamide | 1.32 | 459.2 | 460.3 |
| 162 | 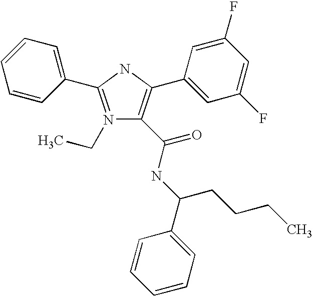 | 4-(3,5-difluorophenyl)-1-eth- yl-2-phenyl-N-(1-phenyl- pentyl)-1H-imidazole-5-car- boxamide | 1.34 | 473.2 | 474.3 |
| 163 | 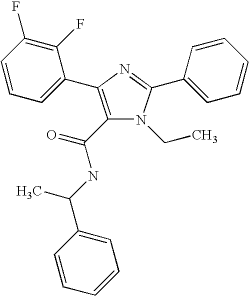 | 4-(2,3-difluorophenyl)-1-eth- yl-2-phenyl-N-(1-phenyl- ethyl)-1H-imidazole-5-car- boxamide | 1.25 | 431.2 | 432.3 |
| 164 | 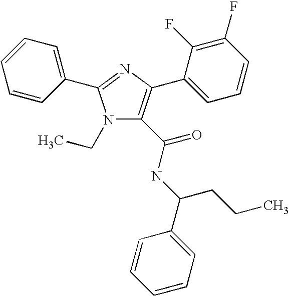 | 4-(2,3-difluorophenyl)-1-eth- yl-2-phenyl-N-(1-phenyl- butyl)-1H-imidazole-5-car- boxamide | 1.28 | 459.2 | 460.3 |
| 165 | 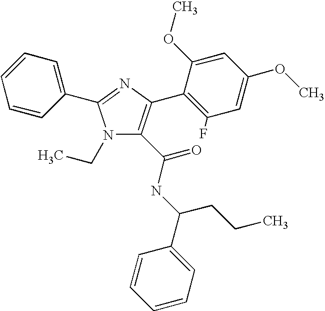 | 1-ethyl-4-(2-fluoro-4,6-di- methoxyphenyl)-2-phenyl-N-(1-phenyl- butyl)-1H-imi- dazole-5-carboxamide | | | |
| 166 | 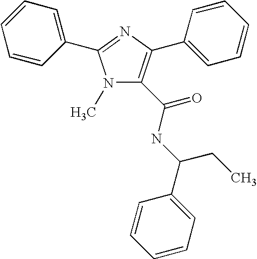 | 1-methyl-2,4-diphenyl-N-(1-phenyl- propyl)-1H-imidazole-5-car- boxamide | 1.2 | 395.2 | 396.2 |
| 167 | 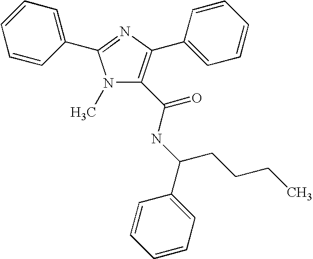 | 1-methyl-2,4-diphenyl-N-(1-phenyl- pentyl)-1H-imidazole-5-car- boxamide | 1.25 | 423.2 | 424.3 |
| 168 | 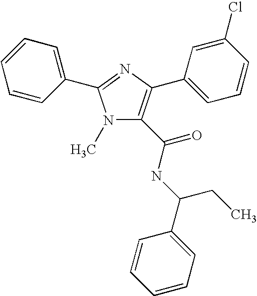 | 4-(3-chlorophenyl)-1-methyl-2-phe- nyl-N-(1-phenylpropyl)-1H-imi- dazole-5-carboxamide | 1.27 | 429.2 | 430.2 |
| 169 | 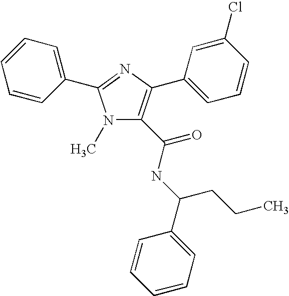 | 4-(3-chlorophenyl)-1-methyl-2-phe- nyl-N-(1-phenylbutyl)-1H-imi- dazole-5-carboxamide | 1.29 | 443.2 | 444.2 |
| 170 | 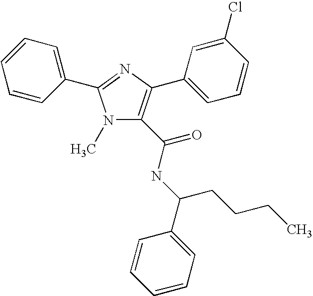 | 4-(3-chlorophenyl)-1-methyl-2-phe- nyl-N-(1-phenylpentyl)-1H-imi- dazole-5-carboxamide | 1.32 | 457.2 | 458.3 |
| 171 | 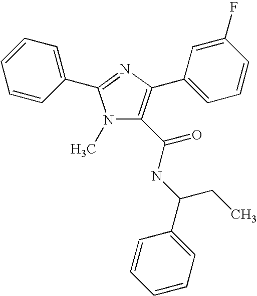 | 4-(3-fluorophenyl)-1-methyl-2-phe- nyl-N-(1-phenylpropyl)-1H-imi- dazole-5-carboxamide | 1.23 | 413.2 | 414.3 |
| 172 | 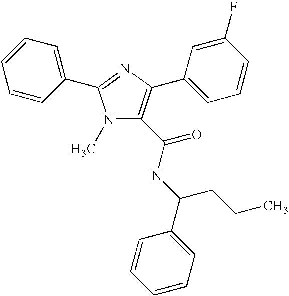 | 4-(3-fluorophenyl)-1-methyl-2-phe- nyl-N-(1-phenylbutyl)-1H-imi- dazole-5-carboxamide | 1.26 | 427.2 | 428.3 |
| 173 | 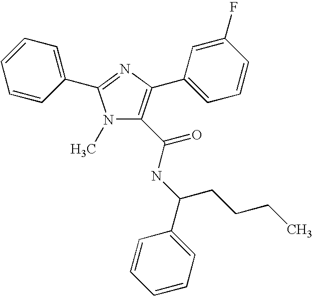 | 4-(3-fluorohenyl)-1-methyl-2-phe- nyl-N-(1-phenylpentyl)-1H-imi- dazole-5-carboxamide | 1.28 | 441.2 | 442.3 |
| 174 | 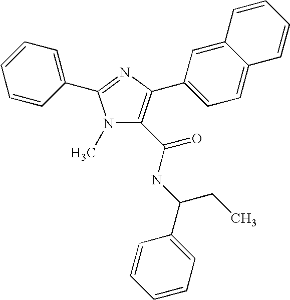 | 1-methyl-4-(2-naphthyl)-2-phe- nyl-N-(1-phenylpropyl)-1H-imi- dazole-5-carboxamide | 1.24 | 445.2 | 446.3 |
| 175 | 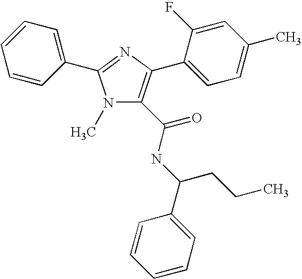 | 4-(2-fluoro-4-methylphenyl)-1-meth- yl-2-phenyl-N-(1-phenyl- butyl)-1H-imidazole-5-car- boxamide | 1.23 | 441.2 | 442.3 |
| 176 | 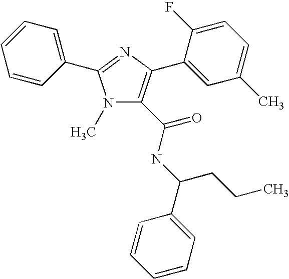 | 4-(2-fluoro-5-methylphenyl)-1-meth- yl-2-phenyl-N-(1-phenyl- butyl)-1H-imidazole-5-car- boxamide | 1.26 | 441.2 | 442.3 |
| 177 | 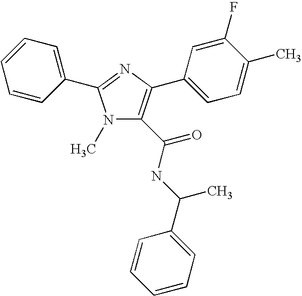 | 4-(3-fluoro-4-methylphenyl)-1-meth- yl-2-phenyl-N-(1-phenyl- ethyl)-1H-imidazole-5-car- boxamide | 1.22 | 413.2 | 414.3 |
| 178 | 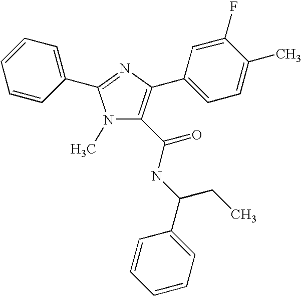 | 4-(3-fluoro-4-methylphenyl)-1-meth- yl-2-phenyl-N-(1-phenyl- propyl)-1H-imidazole-5-car- boxamide | 1.24 | 427.2 | 428.3 |
| 179 | 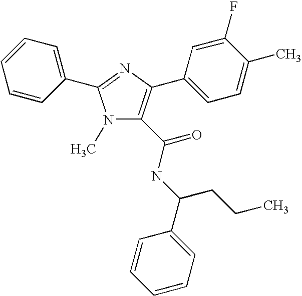 | 4-(3-fluoro-4-methylphenyl)-1-meth- yl-2-phenyl-N-(1-phenyl- butyl)-1H-imidazole-5-car- boxamide | 1.28 | 441.2 | 442.3 |
| 180 | 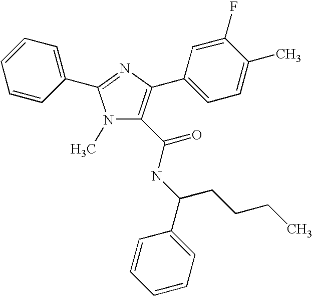 | 4-(3-fluoro-4-methylphenyl)-1-meth- yl-2-phenyl-N-(1-phenyl- pentyl)-1H-imidazole-5-car- boxamide | 1.29 | 455.2 | 456.3 |
| 181 | 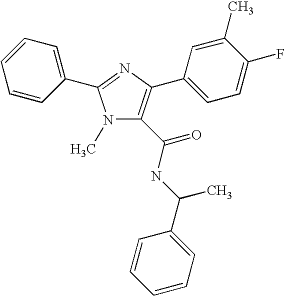 | 4-(4-fluoro-3-methylphenyl)-1-meth- yl-2-phenyl-N-(1-phenyl- ethyl)-1H-imidazole-5-car- boxamide | 1.21 | 413.2 | 414.3 |
| 182 | 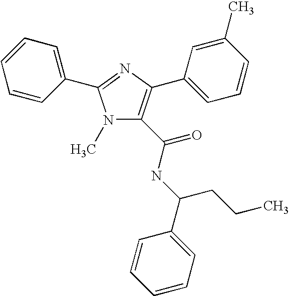 | 1-methyl-4-(3-methylphenyl)-2-phe- nyl-N-(1-phenylbutyl)-1H-imi- dazole-5-carboxamide | 1.25 | 423.2 | 424.3 |
| 183 | 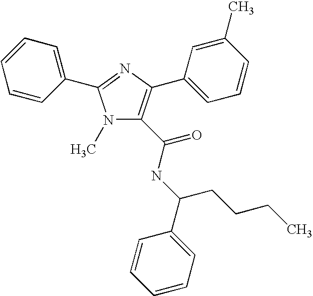 | 1-methyl-4-(3-methylphenyl)-2-phe- nyl-N-(1-phenylpentyl)-1H-imi- dazole-5-carboxamide | 1.27 | 437.2 | 438.3 |
| 184 | 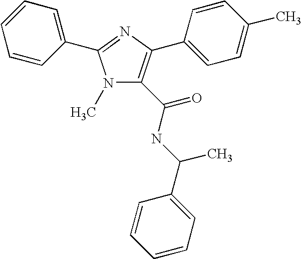 | 1-methyl-4-(4-methylphenyl)-2-phe- nyl-N-(1-phenylethyl)-1H-imi- dazole-5-carboxamide | 1.2 | 395.2 | 396.3 |
| 185 | 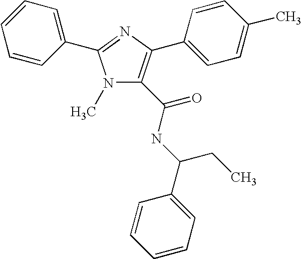 | 1-methyl-4-(4-methylphenyl)-2-phe- nyl-N-(1-phenylpropyl)-1H-imi- dazole-5-carboxamide | 1.21 | 409.2 | 410.3 |
| 186 | 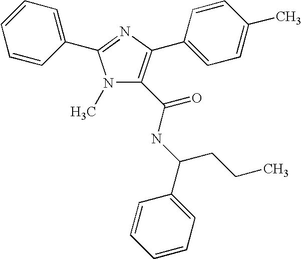 | 1-methyl-4-(4-methylphenyl)-2-phe- nyl-N-(1-phenylbutyl)-1H-imi- dazole-5-carboxamide | 1.24 | 423.2 | 424.3 |
| 187 | 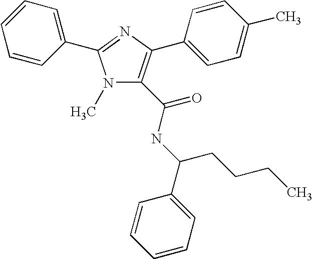 | 1-methyl-4-(4-methylphenyl)-2-phe- nyl-N-(1-phenylpentyl)-1H-imi- dazole-5-carboxamide | 1.26 | 437.2 | 438.3 |
| 188 | 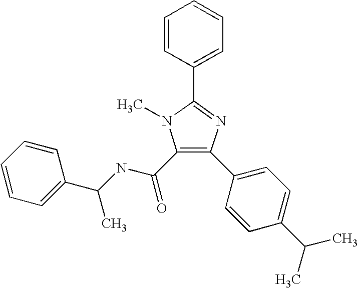 | 4-(4-isopropylphenyl)-1-meth- yl-2-phenyl-N-(1-phenyl- ethyl)-1H-imidazole-5-car- boxamide | 1.25 | 423.2 | 424.3 |
| 189 | 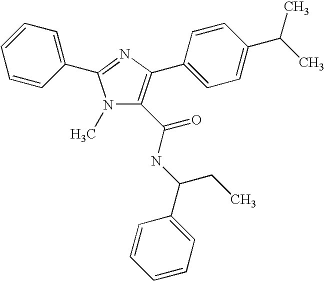 | 4-(4-isopropylphenyl)-1-meth- yl-2-phenyl-N-(1-phenyl- propyl)-1H-imidazole-5-car- boxamide | 1.26 | 437.2 | 438.3 |
| 190 | 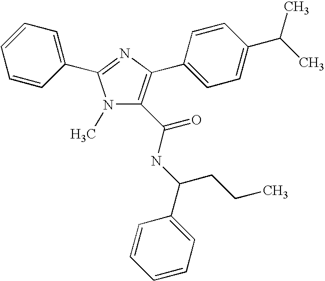 | 4-(4-isopropylphenyl)-1-meth- yl-2-phenyl-N-(1-phenyl- butyl)-1H-imidazole-5-car- boxamide | 1.28 | 451.3 | 452.3 |
| 191 | 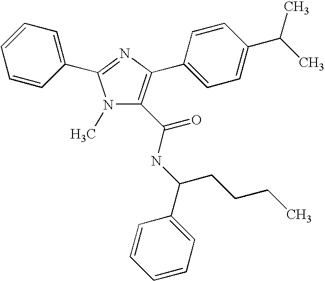 | 4-(4-isopropylphenyl)-1-meth- yl-2-phenyl-N-(1-phenyl- pentyl)-1H-imidazole-5-car- boxamide | 1.3 | 465.3 | 466.4 |
| 192 | 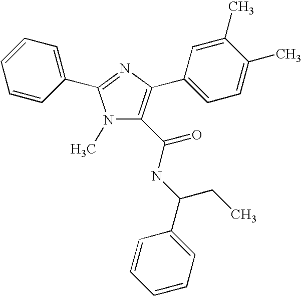 | 4-(3,4-dimethylphenyl)-1-meth- yl-2-phenyl-N-(1-phenyl- propyl)-1H-imidazole-5-car- boxamide | 1.23 | 423.2 | 424.3 |
| 193 | 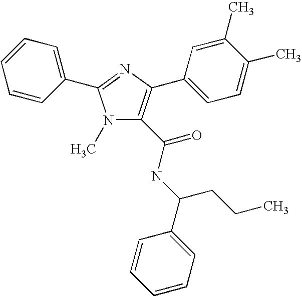 | 4-(3,4-dimethyphenyl)-1-meth- yl-2-phenyl-N-(1-phenyl- butyl)-1H-imidazole-5-car- boxamide | 1.26 | 437.2 | 438.3 |
| 194 | 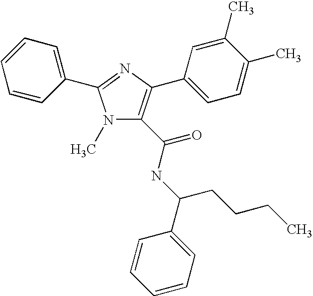 | 4-(3,4-dimethylphenyl)-1-meth- yl-2-phenyl-N-(1-phenyl- pentyl)-1H-imidazole-5-car- boxamide | 1.27 | 451.3 | 452.3 |
| 195 | 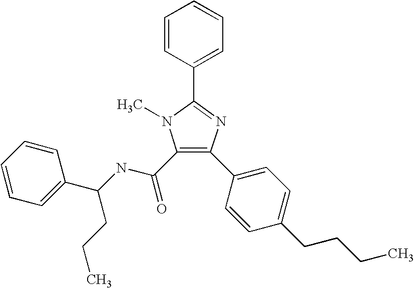 | 4-(4-butylphenyl)-1-methyl-2-phe- nyl-N-(1-phenylbutyl)-1H-imi- dazole-5-carboxamide | 1.32 | 465.3 | 466.4 |
| 196 | 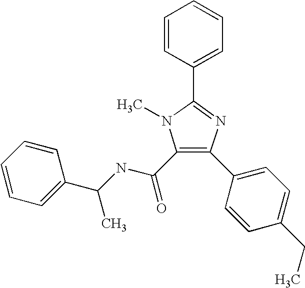 | 4-(4-ethylphenyl)-1-methyl-2-phe- nyl-N-(1-phenylethyl)-1H-imi- dazole-5-carboxamide | 1.22 | 409.2 | 410.3 |
| 197 | 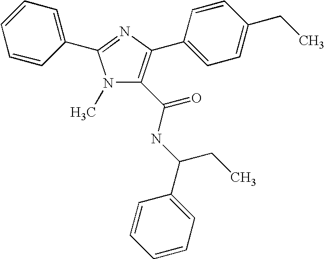 | 4-(4-ethylphenyl)-1-methyl-2-phe- nyl-N-(1-phenylpropyl)-1H-imi- dazole-5-carboxamide | 1.23 | 423.2 | 424.3 |
| 198 | 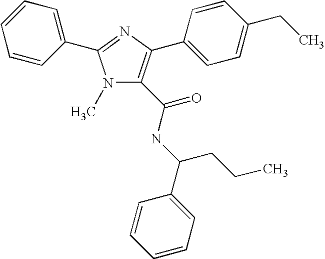 | 4-(4-ethylphenyl)-1-methyl-2-phe- nyl-N-(1-phenylbutyl)-1H-imi- dazole-5-carboxamide | 1.26 | 437.2 | 438.3 |
| 199 | 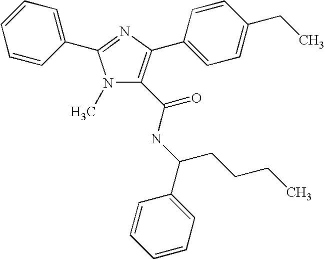 | 4-(4-ethylphenyl)-1-methyl-2-phe- nyl-N-(1-phenylpentyl)-1H-imi- dazole-5-carboxamide | 1.28 | 451.3 | 452.3 |
| 200 | 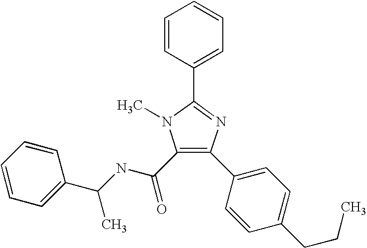 | 1-methyl-2-phenyl-N-(1-phenyl- ethyl)-4-(4-propyl- phenyl)-1H-imidazole-5-car- boxamide | 1.25 | 423.2 | 424.3 |

TABLE I

| | | | Ret. | Calcd. | Obsd. |
|---|---|---|---|---|---|
| | Compound | Name | Time | Mass | M + H |
| 201 | 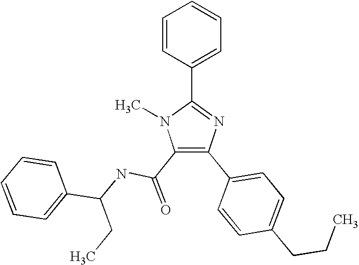 | 1-methyl-2-phenyl-N-(1- phenylpropyl)-4-(4- propylphenyl)-1H-imidazole- 5-carboxamide | 1.26 | 437.2 | 438.3 |
| 202 | 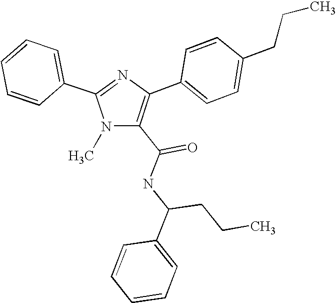 | 1-methyl-2-phenyl-N-(1- phenylbutyl)-4-(4- propylphenyl)-1H-imidazole- 5-carboxamide | 1.29 | 451.3 | 452.3 |
| 203 | 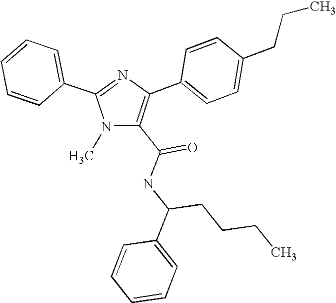 | 1-methyl-2-phenyl-N-(1- phenylpentyl)-4-(4- propylphenyl)-1H-imidazole- 5-carboxamide | 1.32 | 465.3 | 466.4 |
| 204 | 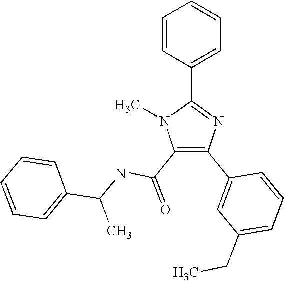 | 4-(3-ethylphenyl)-1-methyl-2- phenyl-N-(1-phenylethyl)-1H- imidazole-5-carboxamide | 1.23 | 409.2 | 410.3 |
| 205 | 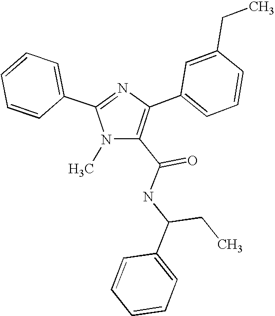 | 4-(3-ethylphenyl)-1-methyl-2- phenyl-N-(1-phenylpropyl)- 1H-imidazole-5-carboxamide | 1.24 | 423.2 | 424.3 |
| 206 | 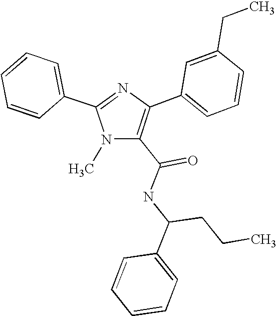 | 4-(3-ethylphenyl)-1-methyl-2- phenyl-N-(1-phenylbutyl)-1H- imidazole-5-carboxamide | 1.27 | 437.2 | 438.3 |
| 207 | 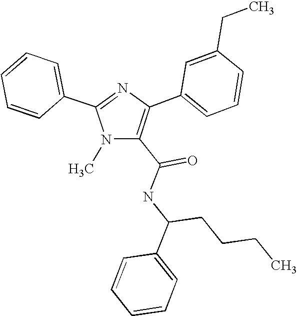 | 4-(3-ethylphenyl)-1-methyl-2- phenyl-N-(1-phenylpentyl)- 1H-imidazole-5-carboxamide | 1.29 | 451.3 | 452.3 |
| 208 | 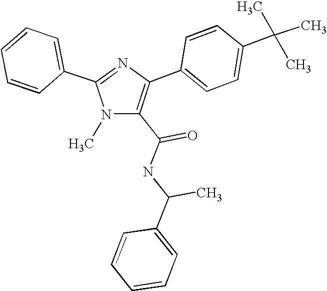 | 4-(4-tert-butylphenyl)-1- methyl-2-phenyl-N-(1- phenylethyl)-1H-imidazole-5- carboxamide | 1.26 | 437.2 | 438.3 |
| 209 | 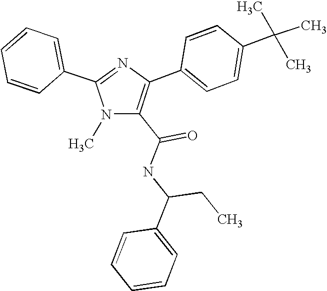 | 4-(4-tert-butylphenyl)-1- methyl-2-phenyl-N-(1- phenylpropyl)-1H-imidazole- 5-carboxamide | 1.27 | 451.3 | 452.3 |
| 210 | 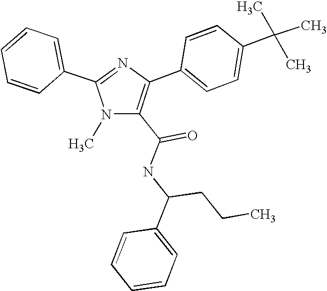 | 4-(4-tert-butylphenyl)-1- methyl-2-phenyl-N-(1- phenylbutyl)-1H-imidazole-5- carboxamide | 1.3 | 465.3 | 466.4 |
| 211 | 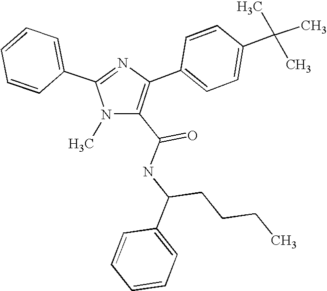 | 4-(4-tert-butylphenyl)-1- methyl-2-phenyl-N-(1- phenylpentyl)-1H-imidazole- 5-carboxamide | 1.32 | 479.3 | 480.4 |
| 212 | 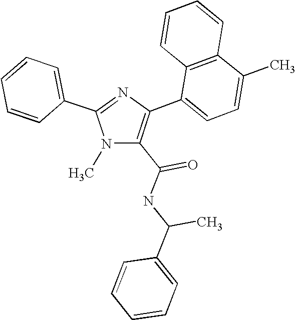 | 1-methyl-4-(4-methyl-1- naphthyl)-2-phenyl-N-(1- phenylethyl)-1H-imidazole-5- carboxamide | 1.24 | 445.2 | 446.3 |
| 213 | 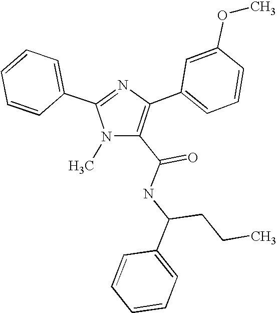 | 4-(3-methoxyphenyl)-1- methyl-2-phenyl-N-(1- phenylbutyl)-1H-imidazole-5- carboxamide | 1.23 | 439.2 | 440.3 |
| 214 | 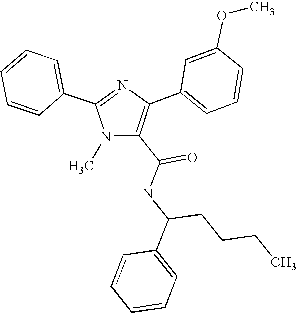 | 4-(3-methoxyphenyl)-1- methyl-2-phenyl-N-(1- phenylpentyl)-1H-imidazole- 5-carboxamide | 1.26 | 453.2 | 454.3 |
| 215 | 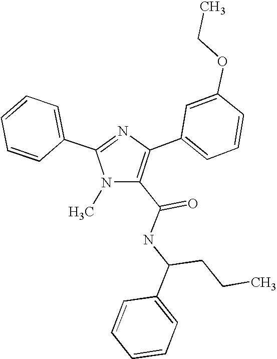 | 4-(3-ethoxyphenyl)-1-methyl- 2-phenyl-N-(1-phenylbutyl)- 1H-imidazole-5-carboxamide | 1.25 | 453.2 | 454.3 |
| 216 | 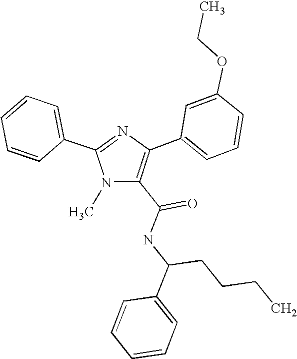 | 4-(3-ethoxyphenyl)-1-methyl- 2-phenyl-N-(1-phenylpentyl)- 1H-imidazole-5-carboxamide | 1.28 | 467.3 | 468.3 |
| 217 | 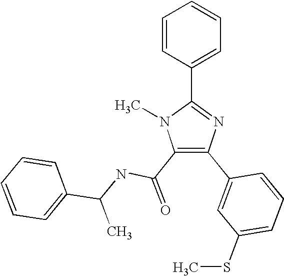 | 1-methyl-4-[3- (methylthio)phenyl]-2-phenyl- N-(1-phenylethyl)-1H- imidazole-5-carboxamide | 1.21 | 427.2 | 428.3 |
| 218 | 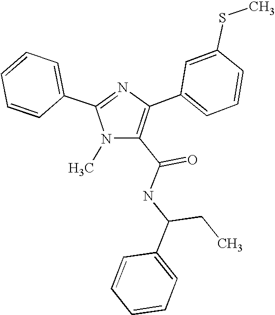 | 1-methyl-4-[3- (methylthio)phenyl]-2-phenyl- N-(1-phenylpropyl)-1H- imidazole-5-carboxamide | 1.23 | 441.2 | 442.3 |
| 219 | 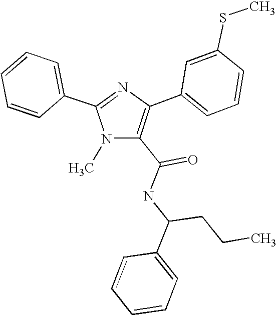 | 1-methyl-4-[3- (methylthio)phenyl]-2-phenyl- N-(1-phenylbutyl)-1H- imidazole-5-carboxamide | 1.26 | 455.2 | 456.3 |
| 220 | 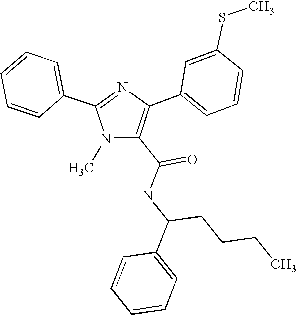 | 1-methyl-4-[3- (methylthio)phenyl]-2-phenyl- N-(1-phenylpentyl)-1H- imidazole-5-carboxamide | 1.28 | 469.2 | 470.3 |
| 221 | 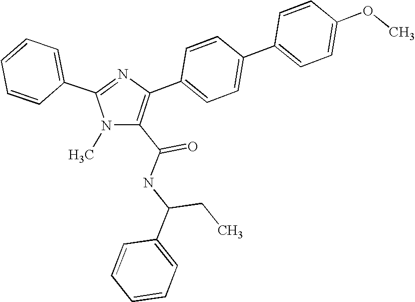 | 4-(4′-methoxybiphenyl-4-yl)- 1-methyl-2-phenyl-N-(1- phenylpropyl)-1H-imidazole- 5-carboxamide | 1.27 | 501.2 | 502.3 |
| 222 | 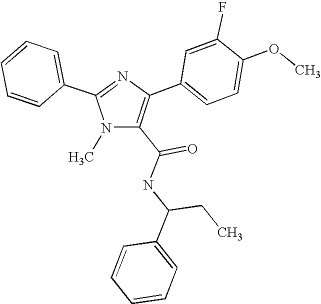 | 4-(3-fluoro-4- methoxyphenyl)-1-methyl-2- phenyl-N-(1-phenylpropyl)- 1H-imidazole-5-carboxamide | 1.2 | 443.2 | 444.3 |
| 223 | 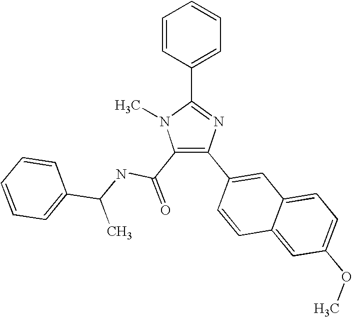 | 4-(6-methoxy-2-naphthyl)-1- methyl-2-phenyl-N-(1- phenylethyl)-1H-imidazole-5- carboxamide | 1.22 | 461.2 | 462.3 |
| 224 | 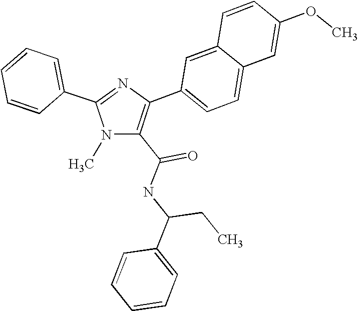 | 4-(6-methoxy-2-naphthyl)-1- methyl-2-phenyl-N-(1- phenylpropyl)-1H-imidazole- 5-carboxamide | 1.24 | 475.2 | 476.3 |
| 225 | 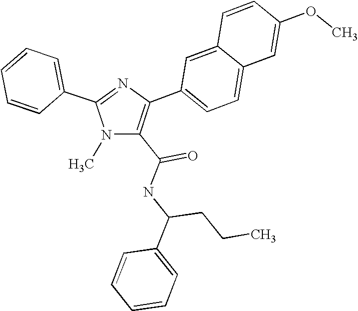 | 4-(6-methoxy-2-naphthyl)-1- methyl-2-phenyl-N-(1- phenylbutyl)-1H-imidazole-5- carboxamide | 1.27 | 489.2 | 490.3 |
| 226 | 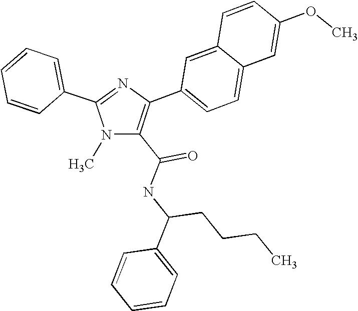 | 4-(6-methoxy-2-naphthyl)-1- methyl-2-phenyl-N-(1- phenylpentyl)-1H-imidazole- 5-carboxamide | 1.28 | 503.3 | 504.3 |
| 227 | 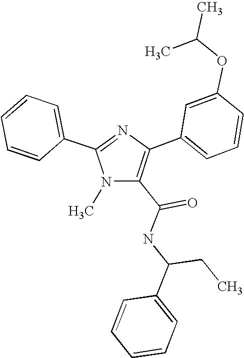 | 4-(3-isopropoxyphenyl)-1- methyl-2-phenyl-N-(1- phenylpropyl)-1H-imidazole- 5-carboxamide | 1.25 | 453.2 | 454.3 |
| 228 | 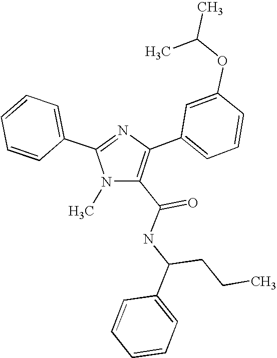 | 4-(3-isopropoxyphenyl)-1- methyl-2-phenyl-N-(1- phenylbutyl)-1H-imidazole-5- carboxamide | 1.27 | 467.3 | 468.3 |
| 229 | 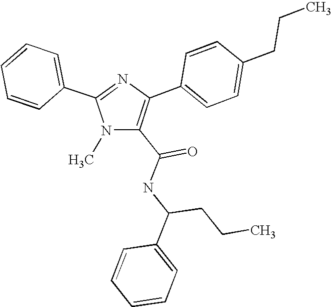 | 4-(4-ethoxyphenyl)-1-methyl- 2-phenyl-N-(1-phenylbutyl)- 1H-imidazole-5-carboxamide | 1.24 | 453.2 | 454.3 |
| 230 | 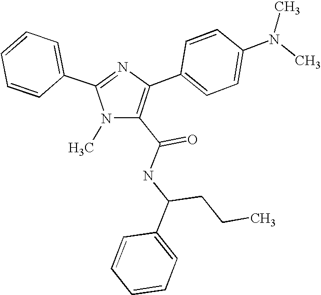 | 4-[4-(dimethylamino)phenyl]- 1-methyl-2-phenyl-N-(1- phenylbutyl)-1H-imidazole-5- carboxamide | | | |
| 231 | 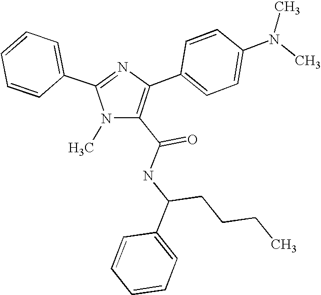 | 4-[4-(dimethylamino)phenyl]- 1-methyl-2-phenyl-N-(1- phenylpentyl)-1H-imidazole- 5-carboxamide | 1.24 | 466.3 | 467.4 |
| 232 | 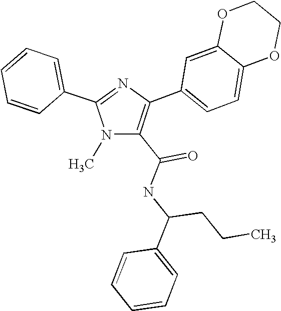 | 4-(2,3-dihydro-1,4- benzodioxin-6-yl)-1-methyl-2- phenyl-N-(1-phenylbutyl)-1H- imidazole-5-carboxamide | 1.21 | 467.2 | 468.3 |
| 233 | 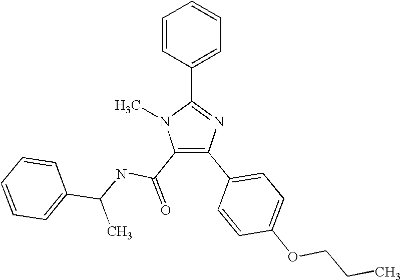 | 1-methyl-2-phenyl-N-(1- phenylethyl)-4-(4- propoxyphenyl)-1H- imidazole-5-carboxamide | 1.23 | 439.2 | 440.3 |
| 234 | 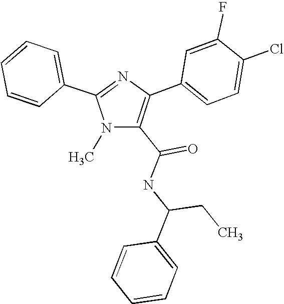 | 4-(4-chloro-3-fluorophenyl)- 1-methyl-2-phenyl-N-(1- phenylpropyl)-1H-imidazole- 5-carboxamide | 1.28 | 447.2 | 448.3 |
| 235 | 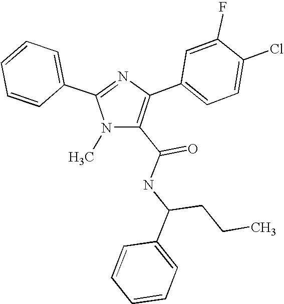 | 4-(4-chloro-3-fluorophenyl)- 1-methyl-2-phenyl-N-(1- phenylbutyl)-1H-imidazole-5- carboxamide | 1.31 | 461.2 | 462.3 |
| 236 | 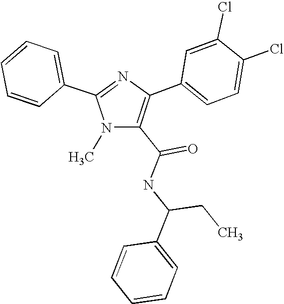 | 4-(3,4-dichlorophenyl)-1- methyl-2-phenyl-N-(1- phenylpropyl)-1H-imidazole- 5-carboxamide | 1.32 | 463.1 | 464.3 |
| 237 | 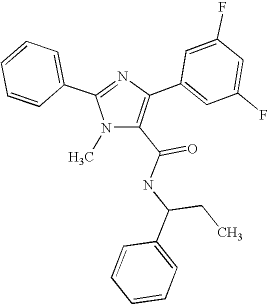 | 4-(3,5-difluorophenyl)-1- methyl-2-phenyl-N-(1- phenylpropyl)-1H-imidazole- 5-carboxamide | 1.27 | 431.2 | 432.3 |
| 238 | 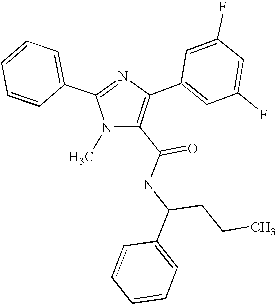 | 4-(3,5-difluorophenyl)-1- methyl-2-phenyl-N-(1- phenylbutyl)-1H-imidazole-5- carboxamide | 1.3 | 445.2 | 446.3 |
| 239 | 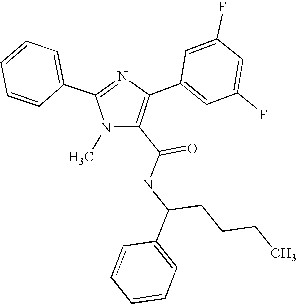 | 4-(3,5-difluorophenyl)-1- methyl-2-phenyl-N-(1- phenylpentyl)-1H-imidazole- 5-carboxamide | 1.33 | 459.2 | 460.3 |
| 240 | 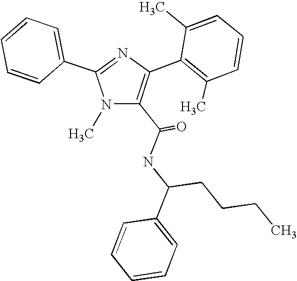 | 4-(2,6-dimethylphenyl)-1- methyl-2-phenyl-N-(1- phenylpentyl)-1H-imidazole- 5-carboxamide | | | |
| 241 | 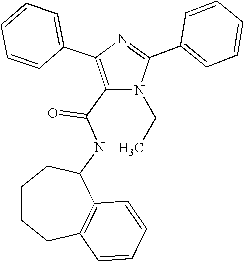 | 1-ethyl-2,4-diphenyl-N- (6,7,8,9-tetrahydro-5H- benzo[7]annulen-5-yl)-1H- imidazole-5-carboxamide | 1.28 | 435.2 | 436.3 |
| 242 | 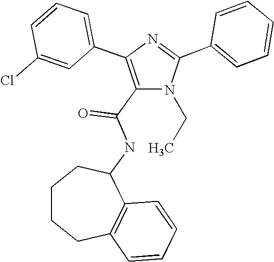 | 4-(3-chlorophenyl)-1-ethyl-2- phenyl-N-(6,7,8,9-tetrahydro- 5H-benzo[7]annulen-5-yl)- 1H-imidazole-5-carboxamide | 1.34 | 469.2 | 470.3 |
| 243 | 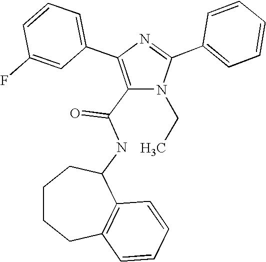 | 1-ethyl-4-(3-fluorophenyl)-2- phenyl-N-(6,7,8,9-tetrahydro- 5H-benzo[7]annulen-5-yl)- 1H-imidazole-5-carboxamide | 1.31 | 453.2 | 454.3 |
| 244 | 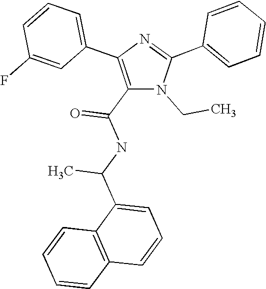 | 1-ethyl-4-(3-fluorophenyl)-N- [1-(1-naphthyl)ethyl]-2- phenyl-1H-imidazole-5- carboxamide | 1.3 | 463.2 | 464.3 |
| 245 | 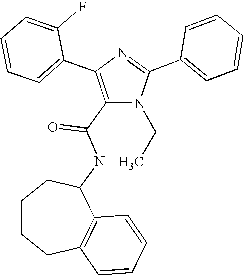 | 1-ethyl-4-(2-fluorophenyl)-2- phenyl-N-(6,7,8,9-tetrahydro- 5H-benzo[7]annulen-5-yl)- 1H-imidazole-5-carboxamide | 1.29 | 453.2 | 454.3 |
| 246 | 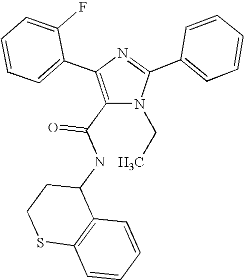 | N-(3,4-dihydro-2H- thiochromen-4-yl)-1-ethyl-4- (2-fluorophenyl)-2-phenyl- 1H-imidazole-5-carboxamide | 1.27 | 457.2 | 458.3 |
| 247 | 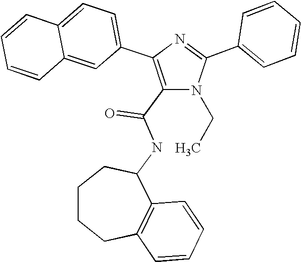 | 1-ethyl-4-(2-naphthyl)-2- phenyl-N-(6,7,8,9-tetrahydro- 5H-benzo[7]annulen-5-yl)- 1H-imidazole-5-carboxamide | 1.32 | 485.2 | 486.3 |
| 248 | 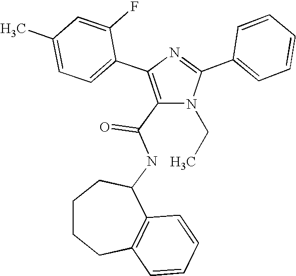 | 1-ethyl-4-(2-fluoro-4- methylphenyl)-2-phenyl-N- (6,7,8,9-tetrahydro-5H- benzo[7]annulen-5-yl)-1H- imidazole-5-carboxamide | 1.3 | 467.2 | 468.3 |
| 249 | 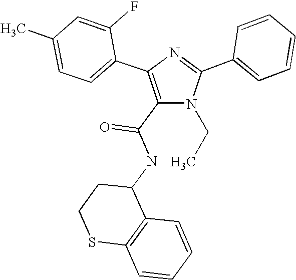 | N-(3,4-dihydro-2H- thiochromen-4-yl)-1-ethyl-4- (2-fluoro-4-methylphenyl)-2- phenyl-1H-imidazole-5- carboxamide | 1.28 | 471.2 | 472.3 |
| 250 | 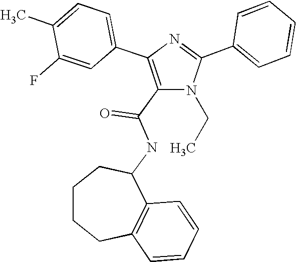 | 1-ethyl-4-(3-fluoro-4- methylphenyl)-2-phenyl-N- (6,7,8,9-tetrahydro-5H- benzo[7]annulen-5-yl)-1H- imidazole-5-carboxamide | 1.32 | 467.2 | 468.3 |
| 251 | 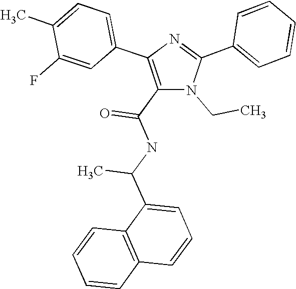 | 1-ethyl-4-(3-fluoro-4- methylphenyl)-N-[1-(1- naphthyl)ethyl]-2-phenyl-1H- imidazole-5-carboxamide | 1.32 | 477.2 | 478.3 |
| 252 | 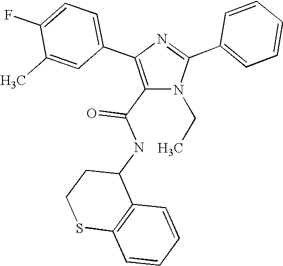 | N-(3,4-dihydro-2H- thiochromen-4-yl)-1-ethyl-4- (4-fluoro-3-methylphenyl)-2- phenyl-1H-imidazole-5- carboxamide | 1.29 | 471.2 | 472.3 |
| 253 | 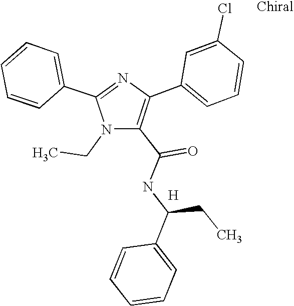 | 4-(3-chlorophenyl)-1-ethyl-2- phenyl-N-[(1S)-1- phenylpropyl]-1H-imidazole- 5-carboxamide | 1.3 | 443.2 | 444.2 |
| 254 | 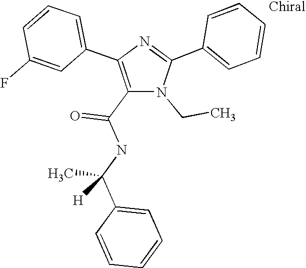 | 1-ethyl-4-(3-fluorophenyl)-2- phenyl-N-[(1S)-1- phenylethyl]-1H-imidazole-5- carboxamide | 1.25 | 413.2 | 414.2 |
| 255 | 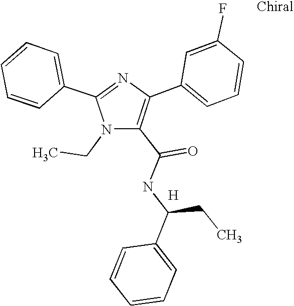 | 1-ethyl-4-(3-fluorophenyl)-2- phenyl-N-[(1S)-1- phenylpropyl]-1H-imidazole- 5-carboxamide | 1.27 | 427.2 | 428.3 |
| 256 | 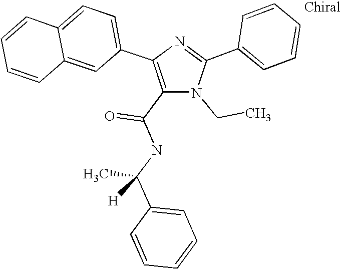 | 1-ethyl-4-(2-naphthyl)-2- phenyl-N-[(1S)-1- phenylethyl]-1H-imidazole-5- carboxamide | 1.27 | 445.2 | 446.3 |
| 257 | 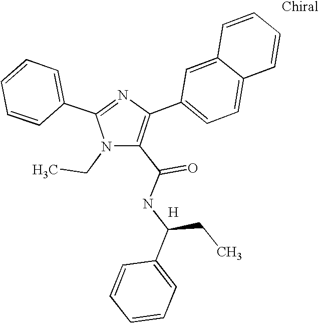 | 1-ethyl-4-(2-naphthyl)-2- phenyl-N-[(1S)-1- phenylpropyl]-1H-imidazole- 5-carboxamide | 1.28 | 459.2 | 460.3 |
| 258 | 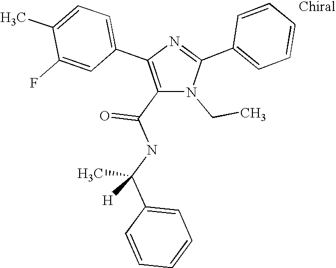 | 1-ethyl-4-(3-fluoro-4- methylphenyl)-2-phenyl-N- [(1S)-1-phenylethyl]-1H- imidazole-5-carboxamide | 1.26 | 427.2 | 428.2 |
| 259 | 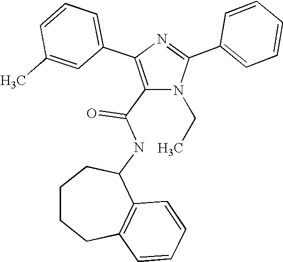 | 1-ethyl-4-(3-methylphenyl)-2- phenyl-N-(6,7,8,9-tetrahydro- 5H-benzo[7]annulen-5-yl)- 1H-imidazole-5-carboxamide | 1.29 | 449.2 | 450.3 |
| 260 | 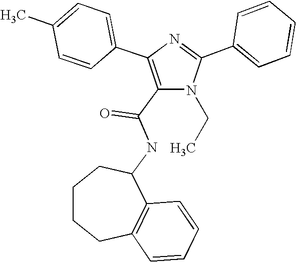 | 1-ethyl-4-(4-methylphenyl)-2- phenyl-N-(6,7,8,9-tetrahydro- 5H-benzo[7]annulen-5-yl)- 1H-imidazole-5-carboxamide | 1.3 | 449.2 | 450.3 |
| 261 | 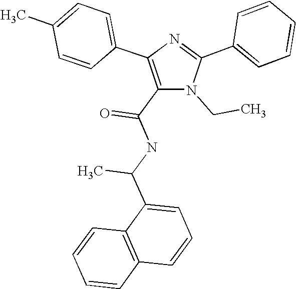 | 1-ethyl-4-(4-methylphenyl)-N- [1-(1-naphthyl)ethyl]-2- phenyl-1H-imidazole-5- carboxamide | 1.29 | 459.2 | 460.3 |
| 262 | 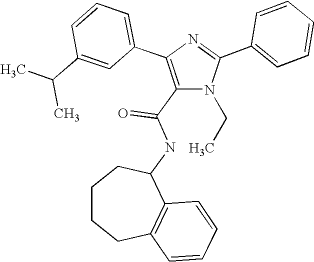 | 1-ethyl-4-(3-isopropylphenyl)- 2-phenyl-N-(6,7,8,9- tetrahydro-5H- benzo[7]annulen-5-yl)-1H- imidazole-5-carboxamide | 1.34 | 477.3 | 478.3 |
| 263 | 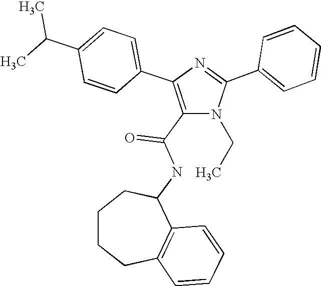 | 1-ethyl-4-(4-isopropylphenyl)- 2-phenyl-N-(6,7,8,9- tetrahydro-5H- benzo[7]annulen-5-yl)-1H- imidazole-5-carboxamide | 1.33 | 477.3 | 478.3 |
| 264 | 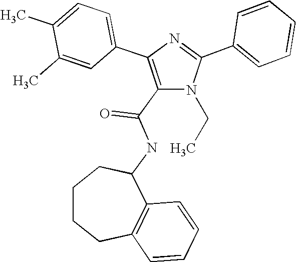 | 4-(3,4-dimethylphenyl)-1- ethyl-2-phenyl-N-(6,7,8,9- tetrahydro-5H- benzo[7]annulen-5-yl)-1H- imidazole-5-carboxamide | 1.3 | 463.3 | 464.3 |
| 265 | 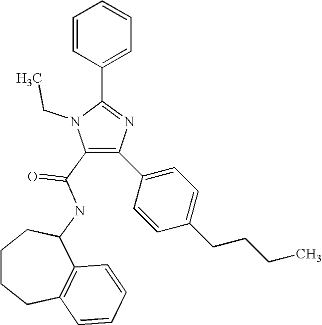 | 4-(4-butylphenyl)-1-ethyl-2- phenyl-N-(6,7,8,9-tetrahydro- 5H-benzo[7]annulen-5-yl)- 1H-imidazole-5-carboxamide | 1.37 | 491.3 | 492.3 |
| 266 | 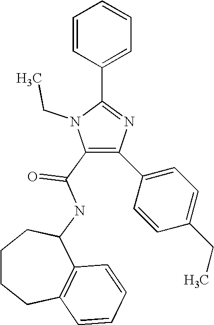 | 1-ethyl-4-(4-ethylphenyl)-2- phenyl-N-(6,7,8,9-tetrahydro- 5H-benzo[7]annulen-5-yl)- 1H-imidazole-5-carboxamide | 1.25 | 463.3 | 464.3 |
| 267 | 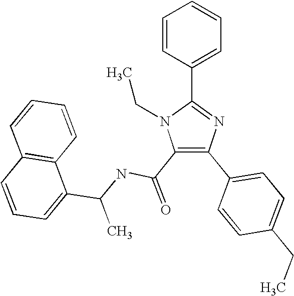 | 1-ethyl-4-(4-ethylphenyl)-N- [1-(1-naphthyl)ethyl]-2- phenyl-1H-imidazole-5- carboxamide | 1.31 | 473.2 | 474.3 |
| 268 | 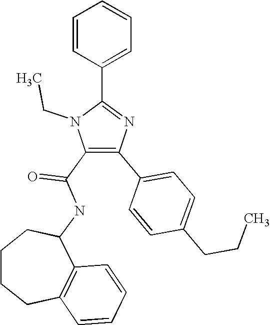 | 1-ethyl-2-phenyl-4-(4- propylphenyl)-N-(6,7,8,9- tetrahydro-5H- benzo[7]annulen-5-yl)-1H- imidazole-5-carboxamide | 1.34 | 477.3 | 478.3 |
| 269 | 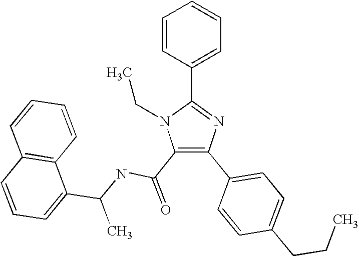 | 1-ethyl-N-[1-(1- naphthyl)ethyl]-2-phenyl-4- (4-propylphenyl)-1H- imidazole-5-carboxamide | 1.34 | 487.3 | 488.3 |
| 270 | 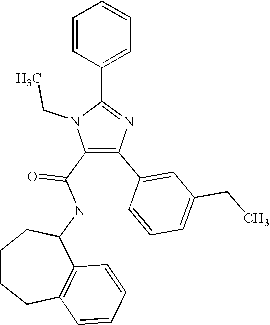 | 1-ethyl-4-(3-ethylphenyl)-2- phenyl-N-(6,7,8,9-tetrahydro- 5H-benzo[7]annulen-5-yl)- 1H-imidazole-5-carboxamide | 1.31 | 463.3 | 464.3 |
| 271 | 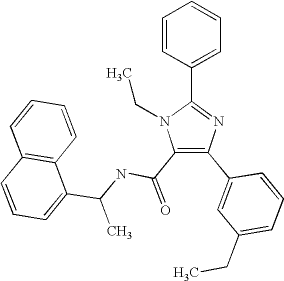 | 1-ethyl-4-(3-ethylphenyl)-N- [1-(1-naphthyl)ethyl]-2- phenyl-1H-imidazole-5- carboxamide | 1.31 | 473.2 | 474.3 |
| 272 | 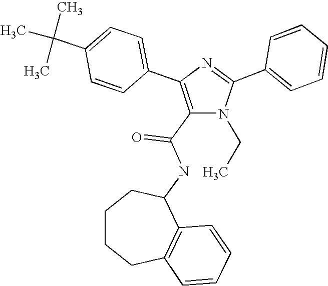 | 4-(4-tert-butylphenyl)-1-ethyl- 2-phenyl-N-(6,7,8,9- tetrahydro-5H- benzo[7]annulen-5-yl)-1H- imidazole-5-carboxamide | 1.36 | 491.3 | 492.3 |
| 273 | 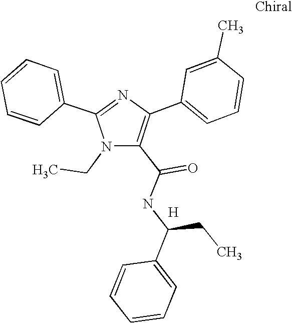 | 1-ethyl-4-(3-methylphenyl)-2- phenyl-N-[(1S)-1- phenylpropyl]-1H-imidazole- 5-carboxamide | 1.26 | 423.2 | 424.2 |
| 274 | 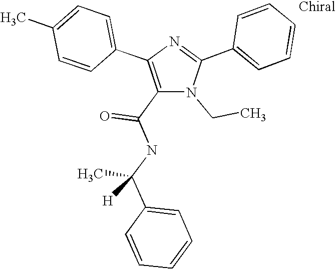 | 1-ethyl-4-(4-methylphenyl)-2- phenyl-N-[(1S)-1- phenylethyl]-1H-imidazole-5- carboxamide | 1.24 | 409.2 | 410.2 |
| 275 | 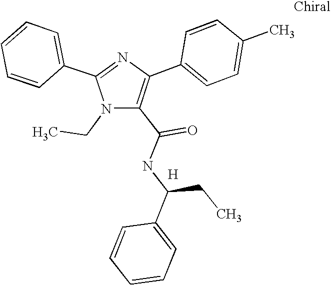 | 1-ethyl-4-(4-methylphenyl)-2- phenyl-N-[(1S)-1- phenylpropyl]-1H-imidazole- 5-carboxamide | 1.26 | 423.2 | 424.2 |
| 276 | 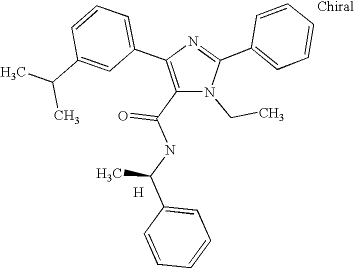 | 1-ethyl-4-(3-isopropylphenyl)- 2-phenyl-N-[(1R)-1- phenylethyl]-1H-imidazole-5- carboxamide | 1.29 | 437.2 | 438.3 |
| 277 | 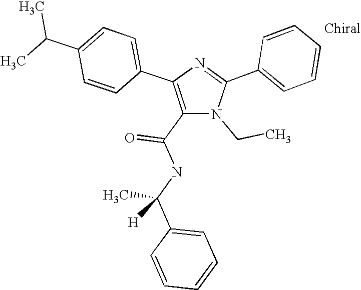 | 1-ethyl-4-(4-isopropylphenyl)- 2-phenyl-N-[(1S)-1- phenylethyl]-1H-imidazole-5- carboxamide | 1.29 | 437.2 | 438.3 |
| 278 | 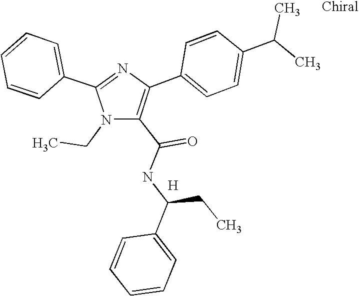 | 1-ethyl-4-(4-isopropylphenyl)- 2-phenyl-N-[(1S)-1- phenylpropyl]-1H-imidazole- 5-carboxamide | 1.3 | 451.3 | 452.3 |
| 279 | 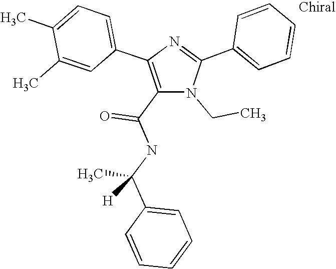 | 4-(3,4-dimethylphenyl)-1- ethyl-2-phenyl-N-[(1S)-1- phenylethyl]-1H-imidazole-5- carboxamide | 1.25 | 423.2 | 424.3 |
| 280 | 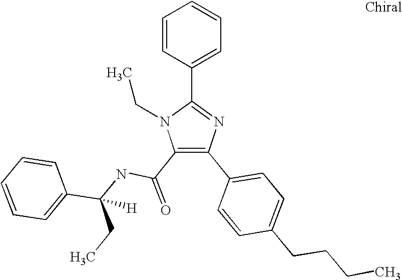 | 4-(4-butylphenyl)-1-ethyl-2- phenyl-N-[(1S)-1- phenylpropyl]-1H-imidazole- 5-carboxamide | 1.34 | 465.3 | 466.3 |
| 281 | 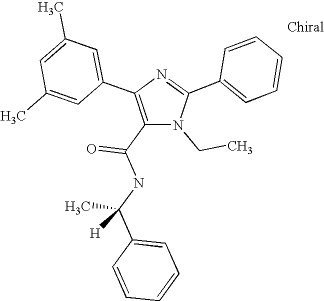 | 4-(3,5-dimethylphenyl)-1- ethyl-2-phenyl-N-[(1S)-1- phenylethyl]-1H-imidazole-5- carboxamide | 1.26 | 423.2 | 424.3 |
| 282 | 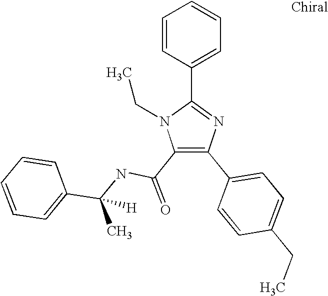 | 1-ethyl-4-(4-ethylphenyl)-2- phenyl-N-[(1S)-1- phenylethyl]-1H-imidazole-5- carboxamide | 1.27 | 423.2 | 424.3 |
| 283 | 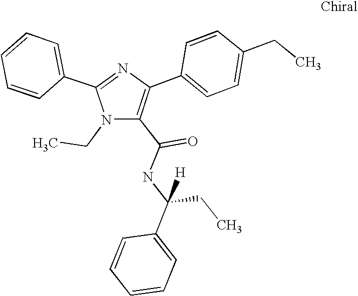 | 1-ethyl-4-(4-ethylphenyl)-2- phenyl-N-[(1R)-1- phenylpropyl]-1H-imidazole- 5-carboxamide | 1.28 | 437.2 | 438.3 |
| 284 | 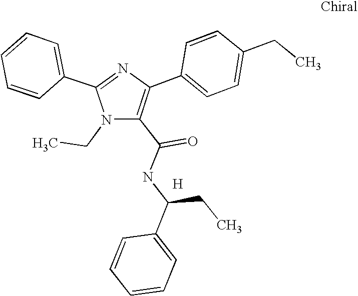 | 1-ethyl-4-(4-ethylphenyl)-2- phenyl-N-[(1S)-1- phenylpropyl]-1H-imidazole- 5-carboxamide | 1.22 | 437.2 | 438.3 |
| 285 | 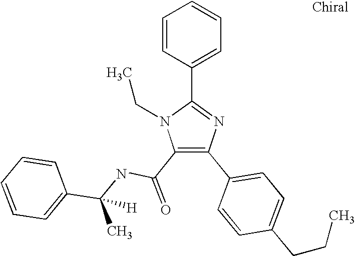 | 1-ethyl-2-phenyl-N-[(1S)-1- phenylethyl]-4-(4- propylphenyl)-1H-imidazole- 5-carboxamide | 1.29 | 437.2 | 438.3 |
| 286 | 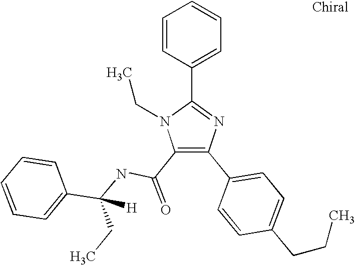 | 1-ethyl-2-phenyl-N-[(1R)-1- phenylpropyl]-4-(4- propylphenyl)-1H-imidazole- 5-carboxamide | 1.31 | 451.3 | 452.3 |
| 287 | 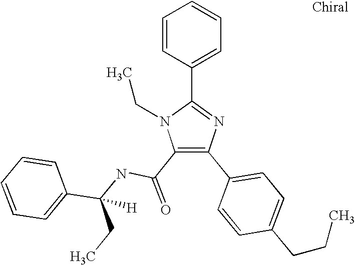 | 1-ethyl-2-phenyl-N-[(1S)-1- phenylpropyl]-4-(4- propylphenyl)-1H-imidazole- 5-carboxamide | 1.31 | 451.3 | 452.3 |
| 288 | 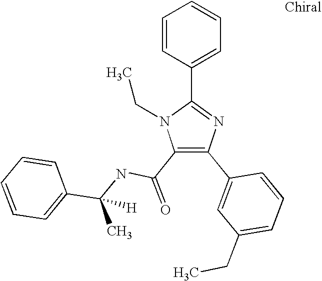 | 1-ethyl-4-(3-ethylphenyl)-2- phenyl-N-[(1S)-1- phenylethyl]-1H-imidazole-5- carboxamide | 1.26 | 423.2 | 424.3 |
| 289 | 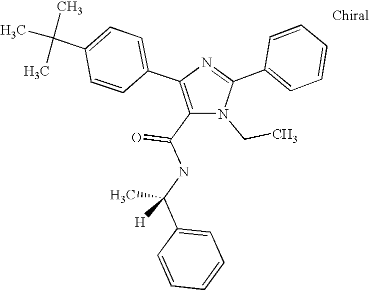 | 4-(4-tert-butylphenyl)-1-ethyl- 2-phenyl-N-[(1S)-1- phenylethyl]-1H-imidazole-5- carboxamide | 1.3 | 451.3 | 452.3 |
| 290 | 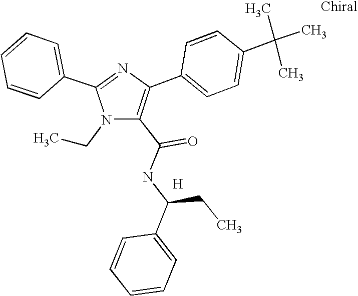 | 4-(4-tert-butylphenyl)-1-ethyl- 2-phenyl-N-[(1S)-1- phenylpropyl]-1H-imidazole- 5-carboxamide | 1.33 | 465.3 | 466.3 |
| 291 | 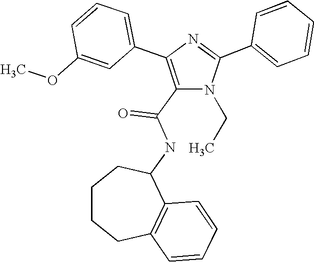 | 1-ethyl-4-(3-methoxyphenyl)- 2-phenyl-N-(6,7,8,9- tetrahydro-5H- benzo[7]annulen-5-yl)-1H- imidazole-5-carboxamide | 1.28 | 465.2 | 466.3 |
| 292 | 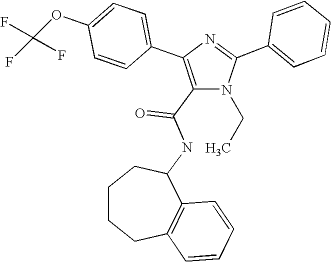 | 1-ethyl-2-phenyl-N-(6,7,8,9- tetrahydro-5H- benzo[7]annulen-5-yl)-4-[4- (trifluoromethoxy)phenyl]- 1H-imidazole-5-carboxamide | 1.37 | 519.2 | 520.2 |
| 293 | 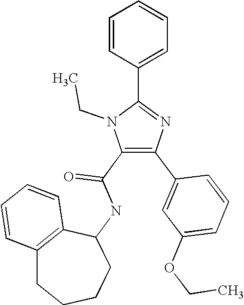 | 4-(3-ethoxyphenyl)-1-ethyl-2- phenyl-N-(6,7,8,9-tetrahydro- 5H-benzo[7]annulen-5-yl)- 1H-imidazole-5-carboxamide | 1.3 | 479.3 | 480.3 |
| 294 | 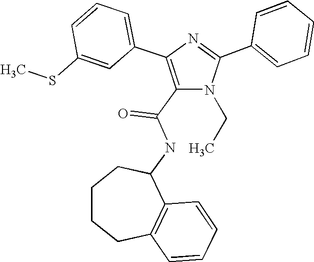 | 1-ethyl-4-[3- (methylthio)phenyl]-2-phenyl- N-(6,7,8,9-tetrahydro-5H- benzo[7]annulen-5-yl)-1H- imidazole-5-carboxamide | 1.31 | 481.2 | 482.3 |
| 295 | 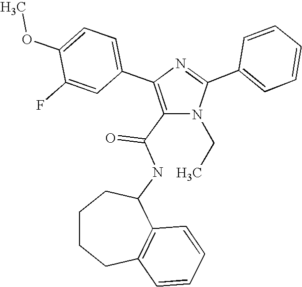 | 1-ethyl-4-(3-fluoro-4- methoxyphenyl)-2-phenyl-N- (6,7,8,9-tetrahydro-5H- benzo[7]annulen-5-yl)-1H- imidazole-5-carboxamide | 1.28 | 483.2 | 484.3 |
| 296 | 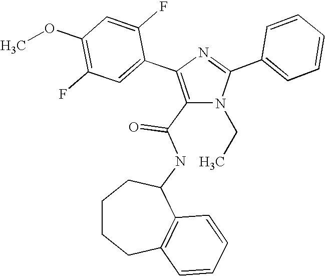 | 4-(2,5-difluoro-4- methoxyphenyl)-1-ethyl-2- phenyl-N-(6,7,8,9-tetrahydro- 5H-benzo[7]annulen-5-yl)- 1H-imidazole-5-carboxamide | 1.3 | 501.2 | 502.3 |
| 297 | 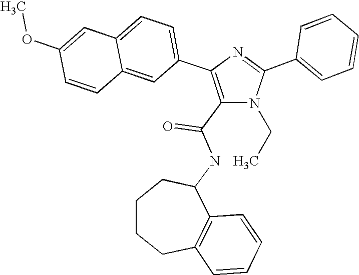 | 1-ethyl-4-(6-methoxy-2- naphthyl)-2-phenyl-N- (6,7,8,9-tetrahydro-5H- benzo[7]annulen-5-yl)-1H- imidazole-5-carboxamide | 1.3 | 515.3 | 516.3 |
| 298 | 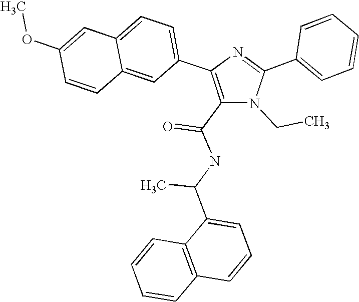 | 1-ethyl-4-(6-methoxy-2- naphthyl)-N-[1-(1- naphthyl)ethyl]-2-phenyl-1H- imidazole-5-carboxamide | 1.31 | 525.2 | 526.3 |
| 299 | 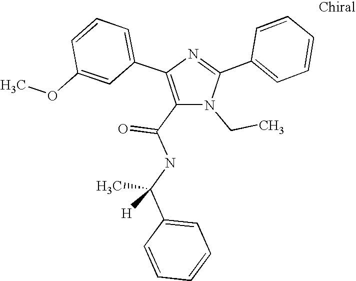 | 1-ethyl-4-(3-methoxyphenyl)- 2-phenyl-N-[(1S)-1- phenylethyl]-1H-imidazole-5- carboxamide | 1.23 | 425.2 | 426.2 |
| 300 | 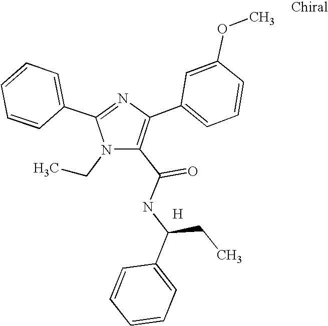 | 1-ethyl-4-(3-methoxyphenyl)- 2-phenyl-N-[(1S)-1- phenylpropyl]-1H-imidazole- 5-carboxamide | 1.25 | 439.2 | 440.3 |
| 301 | 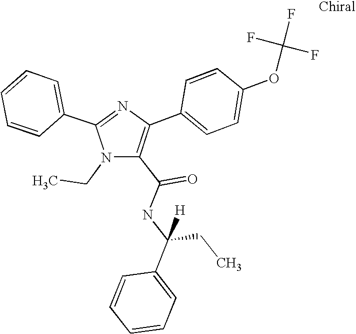 | 1-ethyl-2-phenyl-N-[(1R)-1- phenylpropyl]-4-[4- (trifluoromethoxy)phenyl]- 1H-imidazole-5-carboxamide | 1.33 | 493.2 | 494.2 |
| 302 | 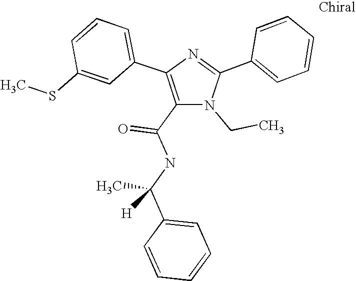 | 1-ethyl-4-[3- (methylthio)phenyl]-2-phenyl- N-[(1S)-1-phenylethyl]-1H- imidazole-5-carboxamide | 1.26 | 441.2 | 442.2 |
| 303 | 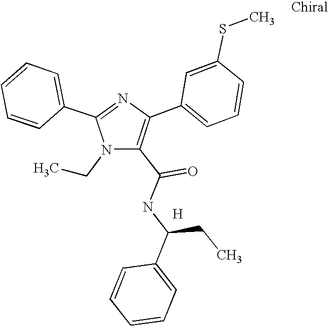 | 1-ethyl-4-[3- (methylthio)phenyl]-2-phenyl- N-[(1S)-1-phenylpropyl]-1H- imidazole-5-carboxamide | 1.28 | 455.2 | 456.2 |
| 304 | 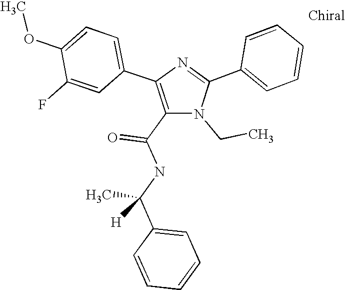 | 1-ethyl-4-(3-fluoro-4- methoxyphenyl)-2-phenyl-N- [(1S)-1-phenylethyl]-1H- imidazole-5-carboxamide | 1.23 | 443.2 | 444.2 |
| 305 | 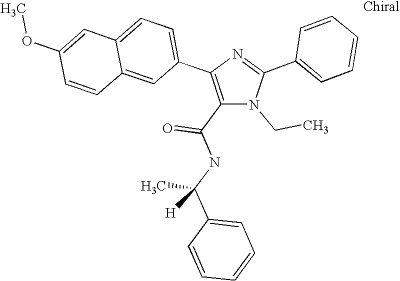 | 1-ethyl-4-(6-methoxy-2- naphthyl)-2-phenyl-N-[(1S)-1- phenylethyl]-1H-imidazole-5- carboxamide | 1.27 | 475.2 | 476.3 |
| 306 | 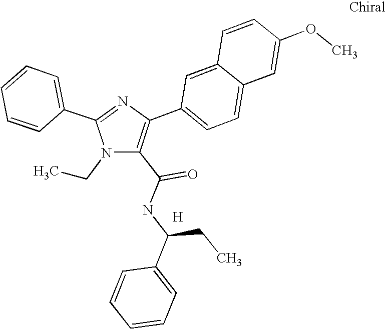 | 1-ethyl-4-(6-methoxy-2- naphthyl)-2-phenyl-N-[(1S)-1- phenylpropyl]-1H-imidazole- 5-carboxamide | 1.28 | 489.2 | 490.3 |
| 307 | 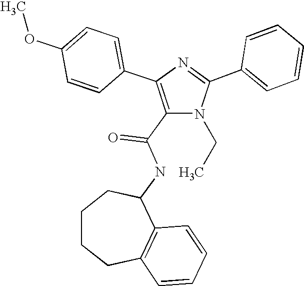 | 1-ethyl-4-(4-methoxyphenyl)- 2-phenyl-N-(6,7,8,9- tetrahydro-5H- benzo[7]annulen-5-yl)-1H- imidazole-5-carboxamide | 1.27 | 465.2 | 466.3 |
| 308 | 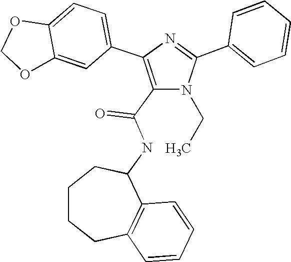 | 4-(1,3-benzodioxol-5-yl)-1- ethyl-2-phenyl-N-(6,7,8,9- tetrahydro-5H- benzo[7]annulen-5-yl)-1H- imidazole-5-carboxamide | 1.26 | 479.2 | 480.3 |
| 309 | 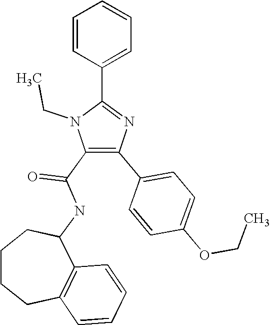 | 4-(4-ethoxyphenyl)-1-ethyl-2- phenyl-N-(6,7,8,9-tetrahydro- 5H-benzo[7]annulen-5-yl)- 1H-imidazole-5-carboxamide | 1.29 | 479.3 | 480.3 |
| 310 | 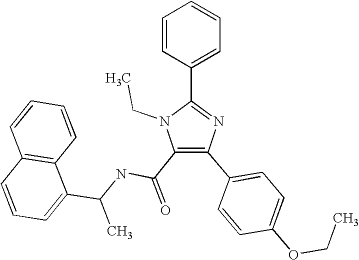 | 4-(4-ethoxyphenyl)-1-ethyl-N- [1-(1-naphthyl)ethyl]-2- phenyl-1H-imidazole-5- carboxamide | 1.29 | 489.2 | 490.3 |
| 311 | 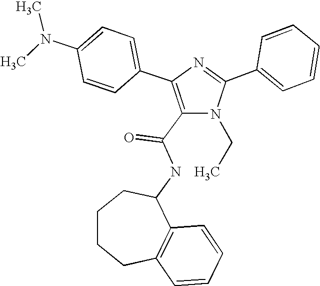 | 4-[4-(dimethylamino)phenyl]- 1-ethyl-2-phenyl-N-(6,7,8,9- tetrahydro-5H- benzo[7]annulen-5-yl)-1H- imidazole-5-carboxamide | 1.27 | 478.3 | 479.3 |
| 312 | 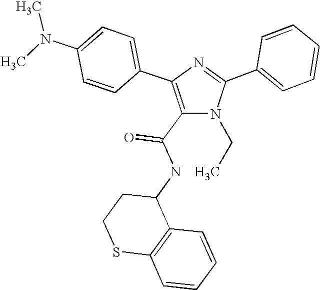 | N-(3,4-dihydro-2H- thiochromen-4-yl)-4-[4- (dimethylamino)phenyl]-1- ethyl-2-phenyl-1H-imidazole- 5-carboxamide | 1.25 | 482.2 | 483.3 |
| 313 | 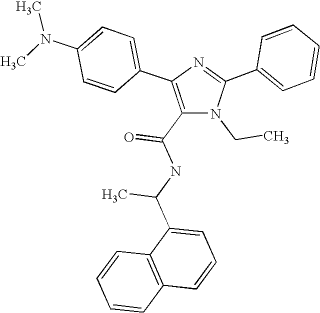 | 4-[4-(dimethylamino)phenyl]- 1-ethyl-N-[1-(1- naphthyl)ethyl]-2-phenyl-1H- imidazole-5-carboxamide | 1.28 | 488.3 | 489.3 |
| 314 | 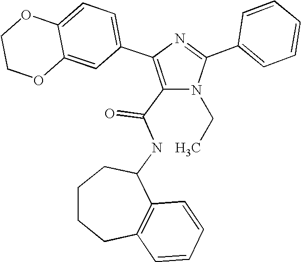 | 4-(2,3-dihydro-1,4- benzodioxin-6-yl)-1-ethyl-2- phenyl-N-(6,7,8,9-tetrahydro- 5H-benzo[7]annulen-5-yl)- 1H-imidazole-5-carboxamide | 1.27 | 493.2 | 494.3 |
| 315 | 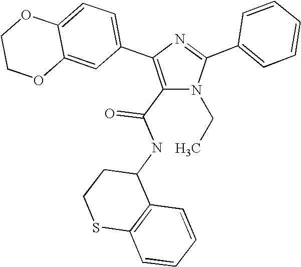 | 4-(2,3-dihydro-1,4- benzodioxin-6-yl)-N-(3,4- dihydro-2H-thiochromen-4- yl)-1-ethyl-2-phenyl-1H- imidazole-5-carboxamide | | | |
| 316 | 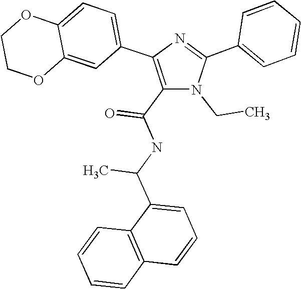 | 4-(2,3-dihydro-1,4- benzodioxin-6-yl)-1-ethyl-N- [1-(1-naphthyl)ethyl]-2- phenyl-1H-imidazole-5- carboxamide | 1.26 | 503.2 | 504.3 |
| 317 | 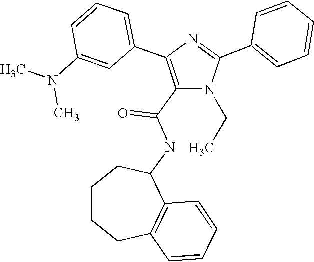 | 4-[3-(dimethylamino)phenyl]- 1-ethyl-2-phenyl-N-(6,7,8,9- tetrahydro-5H- benzo[7]annulen-5-yl)-1H- imidazole-5-carboxamide | 1.27 | 478.3 | 479.3 |
| 318 | 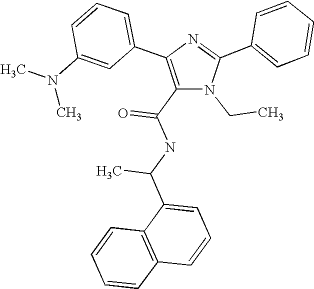 | 4-[3-(dimethylamino)phenyl]- 1-ethyl-N-[1-(1- naphthyl)ethyl]-2-phenyl-1H- imidazole-5-carboxamide | 1.27 | 488.3 | 489.3 |
| 319 | 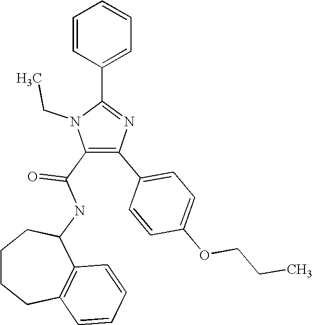 | 1-ethyl-2-phenyl-4-(4- propoxyphenyl)-N-(6,7,8,9- tetrahydro-5H- benzo[7]annulen-5-yl)-1H- imidazole-5-carboxamide | 1.32 | 493.3 | 494.3 |
| 320 | 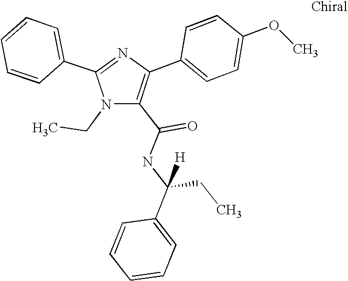 | 1-ethyl-4-(4-methoxyphenyl)- 2-phenyl-N-[(1R)-1- phenylpropyl]-1H-imidazole- 5-carboxamide | 1.24 | 439.2 | 440.2 |
| 321 | 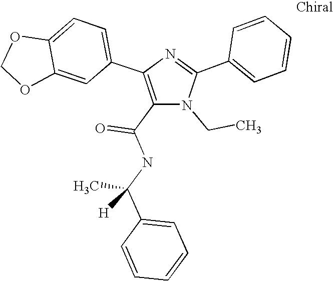 | 4-(1,3-benzodioxol-5-yl)-1- ethyl-2-phenyl-N-[(1S)-1- phenylethyl]-1H-imidazole-5- carboxamide | 1.22 | 439.2 | 440.2 |
| 322 | 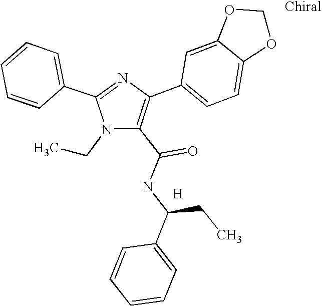 | 4-(1,3-benzodioxol-5-yl)-1- ethyl-2-phenyl-N-[(1S)-1- phenylpropyl]-1H-imidazole- 5-carboxamide | 1.24 | 453.2 | 454.2 |
| 323 | 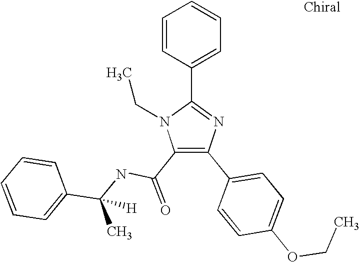 | 4-(4-ethoxyphenyl)-1-ethyl-2- phenyl-N-[(1S)-1- phenylethyl]-1H-imidazole-5- carboxamide | 1.24 | 439.2 | 440.2 |
| 324 | 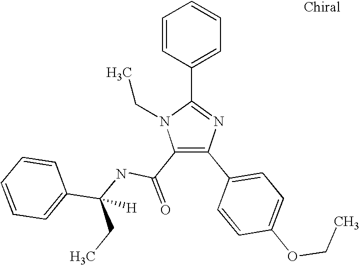 | 4-(4-ethoxyphenyl)-1-ethyl-2- phenyl-N-[(1S)-1- phenylpropyl]-1H-imidazole- 5-carboxamide | 1.27 | 453.2 | 454.3 |
| 325 | 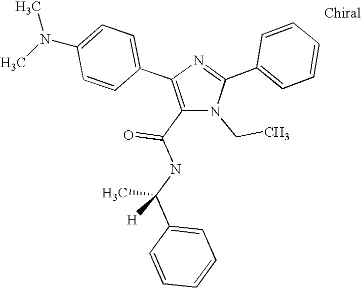 | 4-[4-(dimethylamino)phenyl]- 1-ethyl-2-phenyl-N-[(1S)-1- phenylethyl]-1H-imidazole-5- carboxamide | 1.22 | 438.2 | 439.3 |
| 326 | 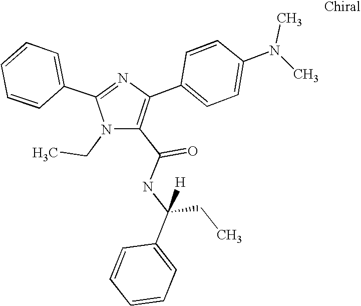 | 4-[4-(dimethylamino)phenyl]- 1-ethyl-2-phenyl-N-[(1R)-1- phenylpropyl]-1H-imidazole- 5-carboxamide | 1.24 | 452.3 | 453.3 |
| 327 | 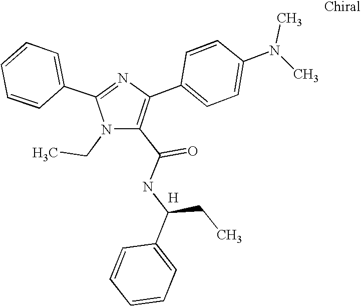 | 4-[4-(dimethylamino)phenyl]- 1-ethyl-2-phenyl-N-[(1S)-1- phenylpropyl]-1H-imidazole- 5-carboxamide | 1.25 | 452.3 | 453.3 |
| 328 | 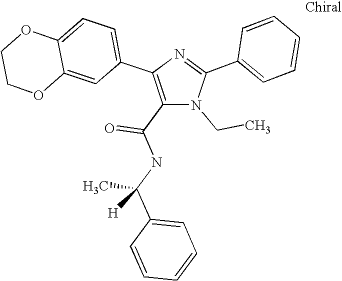 | 4-(2,3-dihydro-1,4- benzodioxin-6-yl)-1-ethyl-2- phenyl-N-[(1S)-1- phenylethyl]-1H-imidazole-5- carboxamide | 1.22 | 453.2 | 454.2 |
| 329 | 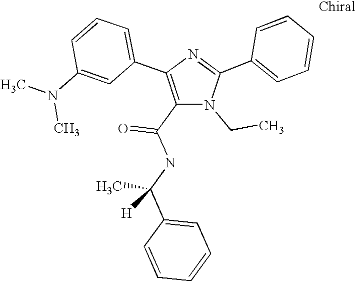 | 4-[3-(dimethylamino)phenyl]- 1-ethyl-2-phenyl-N-[(1S)-1- phenylethyl]-1H-imidazole-5- carboxamide | 1.22 | 438.2 | 439.3 |
| 330 | 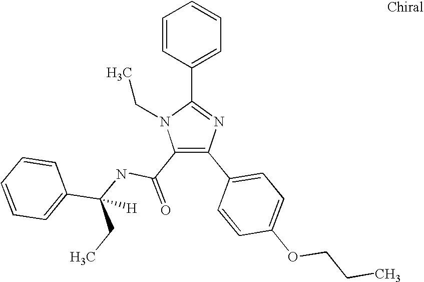 | 1-ethyl-2-phenyl-N-[(1S)-1- phenylpropyl]-4-(4- propoxyphenyl)-1H- imidazole-5-carboxamide | 1.3 | 467.3 | 468.3 |
| 331 | 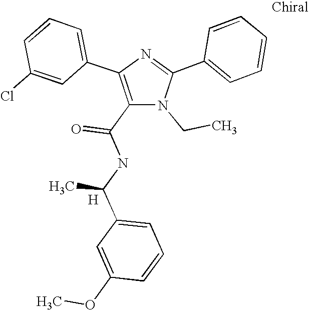 | 4-(3-chlorophenyl)-1-ethyl-N- [(1R)-1-(3- methoxyphenyl)ethyl]-2- phenyl-1H-imidazole-5- carboxamide | 1.22 | 459.2 | 460.3 |
| 332 | 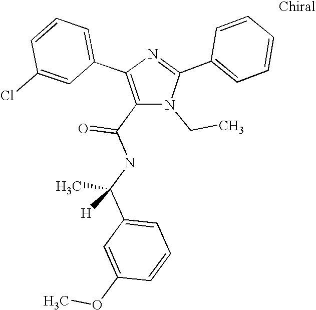 | 4-(3-chlorophenyl)-1-ethyl-N- [(1S)-1-(3- methoxyphenyl)ethyl]-2- phenyl-1H-imidazole-5- carboxamide | 1.21 | 459.2 | 460.3 |
| 333 | 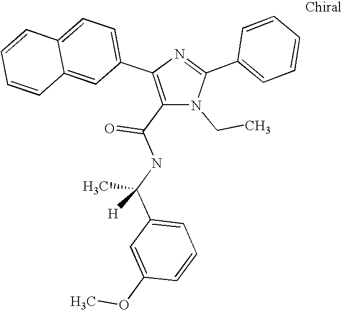 | 1-ethyl-N-[(1S)-1-(3- methoxyphenyl)ethyl]-4-(2- naphthyl)-2-phenyl-1H- imidazole-5-carboxamide | 1.2 | 475.2 | 476.3 |
| 334 | 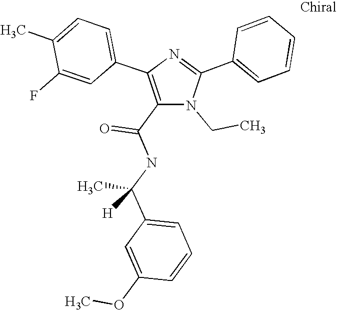 | 1-ethyl-4-(3-fluoro-4- methylphenyl)-N-[(1S)-1-(3- methoxyphenyl)ethyl]-2- phenyl-1H-imidazole-5- carboxamide | 1.2 | 457.2 | 458.3 |
| 335 | 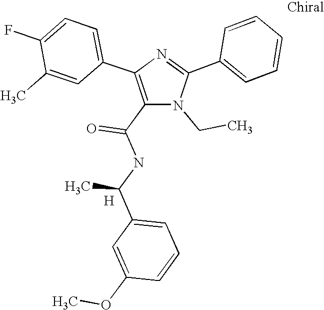 | 1-ethyl-4-(4-fluoro-3- methylphenyl)-N-[(1R)-1-(3- methoxyphenyl)ethyl]-2- phenyl-1H-imidazole-5- carboxamide | 1.2 | 457.2 | 458.3 |
| 336 | 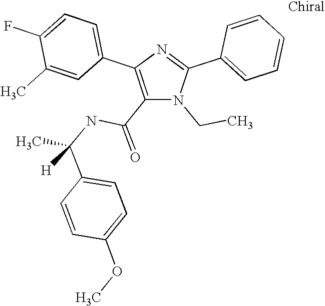 | 1-ethyl-4-(4-fluoro-3- methylphenyl)-N-[(1S)-1-(4- methoxyphenyl)ethyl]-2- phenyl-1H-imidazole-5- carboxamide | 1.19 | 457.2 | 458.3 |
| 337 | 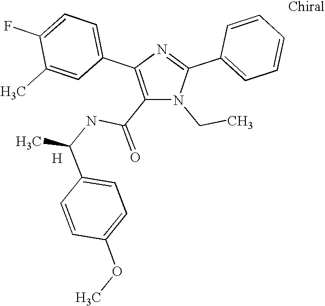 | 1-ethyl-4-(4-fluoro-3- methylphenyl)-N-[(1R)-1-(4- methoxyphenyl)ethyl]-2- phenyl-1H-imidazole-5- carboxamide | 1.19 | 457.2 | 458.3 |
| 338 | 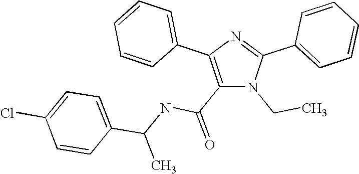 | N-[1-(4-chlorophenyl)ethyl]- 1-ethyl-2,4-diphenyl-1H- imidazole-5-carboxamide | 1.2 | 429.2 | 430.3 |
| 339 | 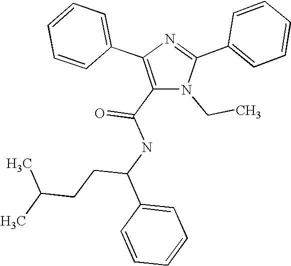 | 1-ethyl-N-(4-methyl-1- phenylpentyl)-2,4-diphenyl- 1H-imidazole-5-carboxamide | 1.26 | 451.3 | 452.4 |
| 340 | 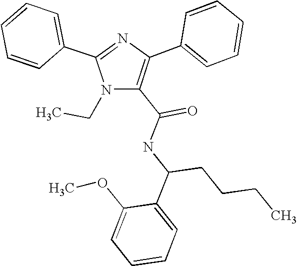 | 1-ethyl-N-[1-(2- methoxyphenyl)pentyl]-2,4- diphenyl-1H-imidazole-5- carboxamide | 1.25 | 467.3 | 468.3 |
| 341 | 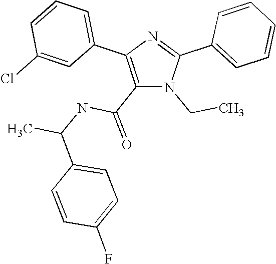 | 4-(3-chlorophenyl)-1-ethyl-N- [1-(4-fluorophenyl)ethyl]-2- phenyl-1H-imidazole-5- carboxamide | 1.23 | 447.2 | 448.3 |
| 342 | 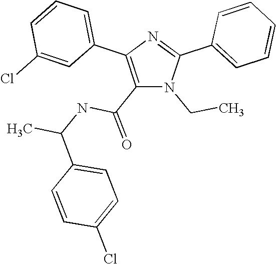 | 4-(3-chlorophenyl)-N-[1-(4- chlorophenyl)ethyl]-1-ethyl-2- phenyl-1H-imidazole-5- carboxamide | 1.26 | 463.1 | 464.2 |
| 343 | 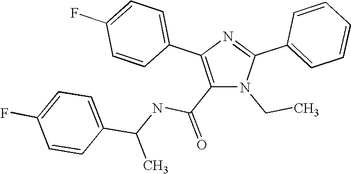 | 1-ethyl-4-(4-fluorophenyl)-N- [1-(4-fluorophenyl)ethyl]-2- phenyl-1H-imidazole-5- carboxamide | 1.18 | 431.2 | 432.3 |
| 344 | 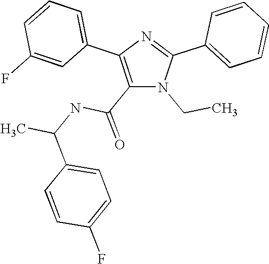 | 1-ethyl-4-(3-fluorophenyl)-N- [1-(4-fluorophenyl)ethyl]-2- phenyl-1H-imidazole-5- carboxamide | 1.19 | 431.2 | 432.3 |
| 345 | 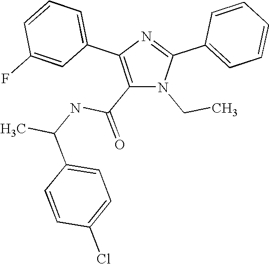 | N-[1-(4-chlorophenyl)ethyl]- 1-ethyl-4-(3-fluorophenyl)-2- phenyl-1H-imidazole-5- carboxamide | 1.22 | 447.2 | 448.3 |
| 346 | 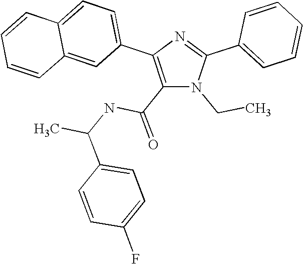 | 1-ethyl-N-[1-(4- fluorophenyl)ethyl]-4-(2- naphthyl)-2-phenyl-1H- imidazole-5-carboxamide | 1.21 | 463.2 | 464.3 |
| 347 | 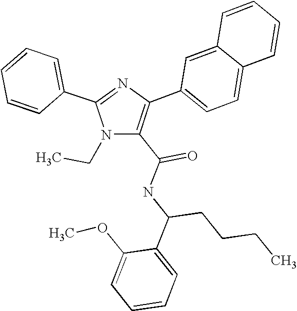 | 1-ethyl-N-[1-(2- methoxyphenyl)pentyl]-4-(2- naphthyl)-2-phenyl-1H- imidazole-5-carboxamide | 1.28 | 517.3 | 518.4 |
| 348 | 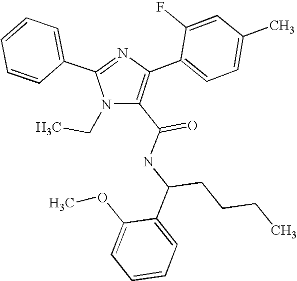 | 1-ethyl-4-(2-fluoro-4- methylphenyl)-N-[1-(2- methoxyphenyl)pentyl]-2- phenyl-1H-imidazole-5- carboxamide | 1.26 | 499.3 | 500.4 |
| 349 | 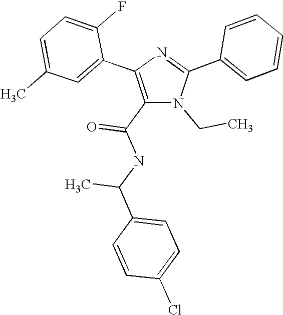 | N-[1-(4-chlorophenyl)ethyl]- 1-ethyl-4-(2-fluoro-5- methylphenyl)-2-phenyl-1H- imidazole-5-carboxamide | 1.22 | 461.2 | 462.3 |
| 350 | 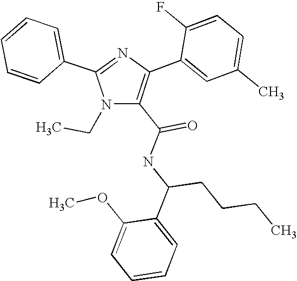 | 1-ethyl-4-(2-fluoro-5- methylphenyl)-N-[1-(2- methoxyphenyl)pentyl]-2- phenyl-1H-imidazole-5- carboxamide | 1.26 | 499.3 | 500.4 |
| 351 | 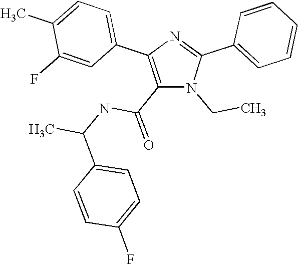 | 1-ethyl-4-(3-fluoro-4- methylphenyl)-N-[1-(4- fluorophenyl)ethyl]-2-phenyl- 1H-imidazole-5-carboxamide | 1.21 | 445.2 | 446.3 |
| 352 | 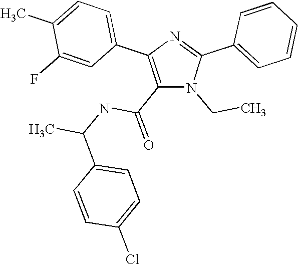 | N-[1-(4-chlorophenyl)ethyl]- 1-ethyl-4-(3-fluoro-4- methylphenyl)-2-phenyl-1H- imidazole-5-carboxamide | 1.24 | 461.2 | 462.3 |
| 353 | 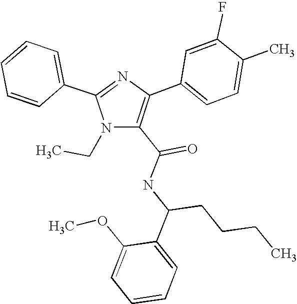 | 1-ethyl-4-(3-fluoro-4- methylphenyl)-N-[1-(2- methoxyphenyl)pentyl]-2- phenyl-1H-imidazole-5- carboxamide | 1.27 | 499.3 | 500.4 |
| 354 | 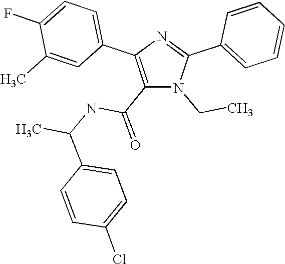 | N-[1-(4-chlorophenyl)ethyl]- 1-ethyl-4-(4-fluoro-3- methylphenyl)-2-phenyl-1H- imidazole-5-carboxamide | 1.23 | 461.2 | 462.3 |
| 355 | 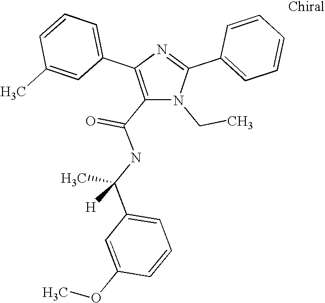 | 1-ethyl-N-[(1S)-1-(3- methoxyphenyl)ethyl]-4-(3- methylphenyl)-2-phenyl-1H- imidazole-5-carboxamide | 1.18 | 439.2 | 440.3 |
| 356 | 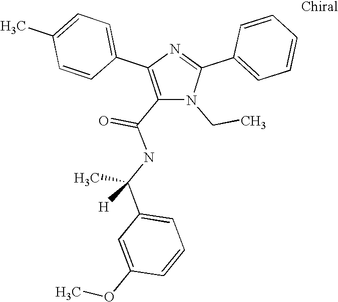 | 1-ethyl-N-[(1S)-1-(3- methoxyphenyl)ethyl]-4-(4- methylphenyl)-2-phenyl-1H- imidazole-5-carboxamide | | | |
| 357 | 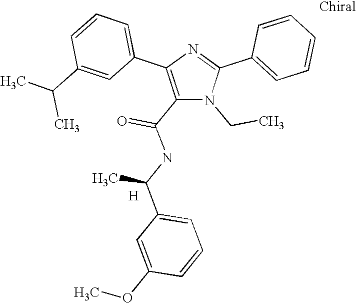 | 1-ethyl-4-(3-isopropylphenyl)- N-[(1R)-1-(3- methoxyphenyl)ethyl]-2- phenyl-1H-imidazole-5- carboxamide | 1.23 | 467.3 | 468.3 |
| 358 | 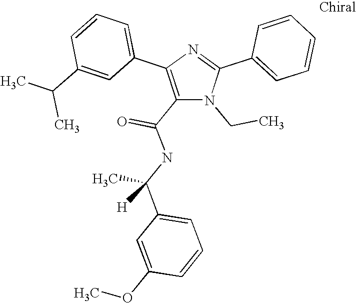 | 1-ethyl-4-(3-isopropylphenyl)- N-[(1S)-1-(3- methoxyphenyl)ethyl]-2- phenyl-1H-imidazole-5- carboxamide | 1.22 | 467.3 | 468.3 |
| 359 | 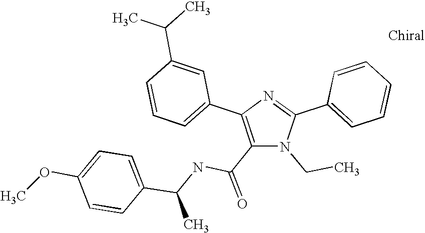 | 1-ethyl-4-(3-isopropylphenyl)- N-[(1S)-1-(4- methoxyphenyl)ethyl]-2- phenyl-1H-imidazole-5- carboxamide | 1.22 | 467.3 | 468.3 |
| 360 | 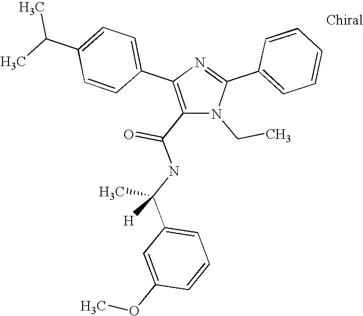 | 1-ethyl-4-(4-isopropylphenyl)- N-[(1S)-1-(3- methoxyphenyl)ethyl]-2- phenyl-1H-imidazole-5- carboxamide | 1.22 | 467.3 | 468.3 |
| 361 | 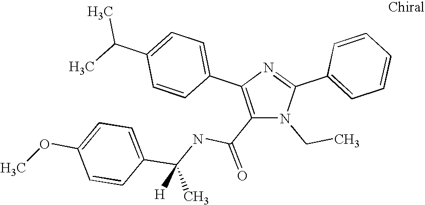 | 1-ethyl-4-(4-isopropylphenyl)- N-[(1R)-1-(4- methoxyphenyl)ethyl]-2- phenyl-1H-imidazole-5- carboxamide | 1.22 | 467.3 | 468.3 |
| 362 | 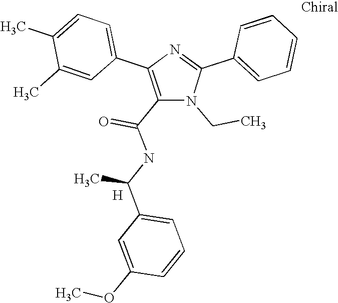 | 4-(3,4-dimethylphenyl)-1- ethyl-N-[(1R)-1-(3- methoxyphenyl)ethyl]-2- phenyl-1H-imidazole-5- carboxamide | 1.19 | 453.2 | 454.3 |
| 363 | 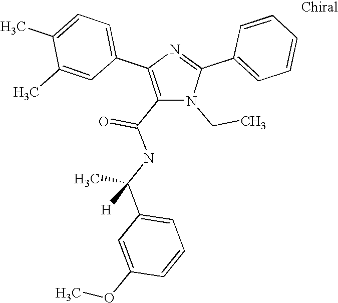 | 4-(3,4-dimethylphenyl)-1- ethyl-N-[(1S)-1-(3- methoxyphenyl)ethyl]-2- phenyl-1H-imidazole-5- carboxamide | 1.2 | 453.2 | 454.3 |
| 364 | 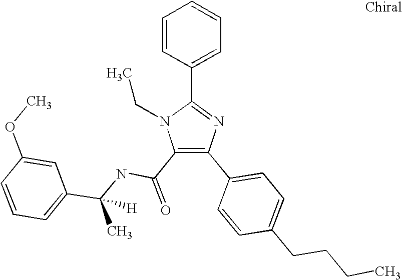 | 4-(4-butylphenyl)-1-ethyl-N- [(1S)-1-(3- methoxyphenyl)ethyl]-2- phenyl-1H-imidazole-5- carboxamide | 1.26 | 481.3 | 482.3 |
| 365 | 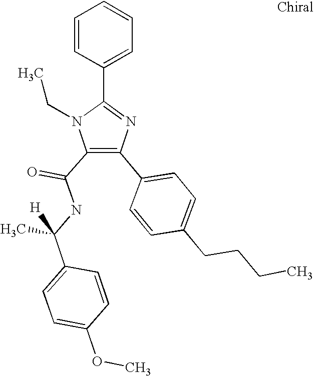 | 4-(4-butylphenyl)-1-ethyl-N- [(1S)-1-(4- methoxyphenyl)ethyl]-2- phenyl-1H-imidazole-5- carboxamide | 1.25 | 481.3 | 482.3 |
| 366 | 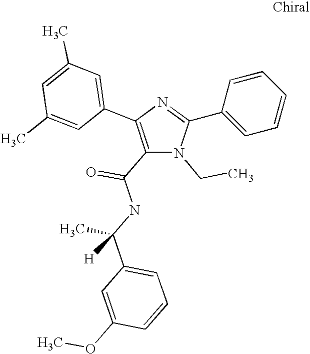 | 4-(3,5-dimethylphenyl)-1- ethyl-N-[(1S)-1-(3- methoxyphenyl)ethyl]-2- phenyl-1H-imidazole-5- carboxamide | 1.2 | 453.2 | 454.3 |
| 367 | 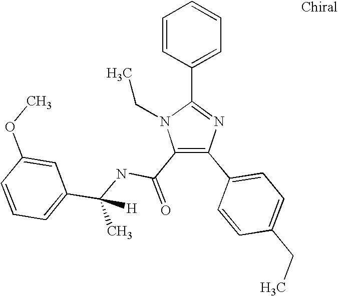 | 1-ethyl-4-(4-ethylphenyl)-N- [(1R)-1-(3- methoxyphenyl)ethyl]-2- phenyl-1H-imidazole-5- carboxamide | 1.2 | 453.2 | 454.3 |
| 368 | 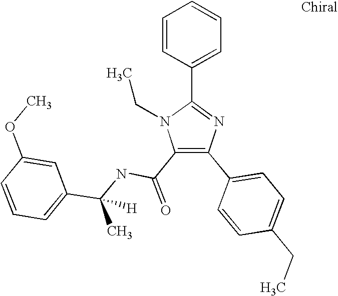 | 1-ethyl-4-(4-ethylphenyl)-N- [(1S)-1-(3- methoxyphenyl)ethyl]-2- phenyl-1H-imidazole-5- carboxamide | 1.21 | 453.2 | 454.3 |
| 369 | 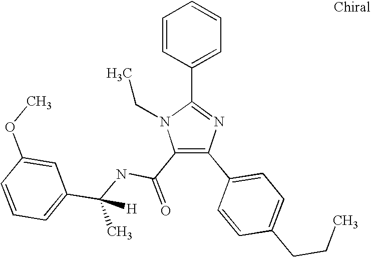 | 1-ethyl-N-[(1R)-1-(3- methoxyphenyl)ethyl]-2- phenyl-4-(4-propylphenyl)- 1H-imidazole-5-carboxamide | 1.23 | 467.3 | 468.3 |
| 370 | 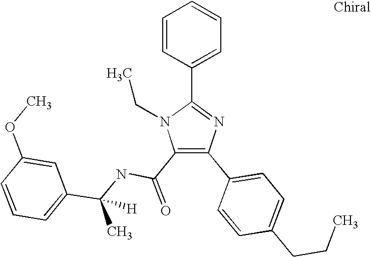 | 1-ethyl-N-[(1S)-1-(3- methoxyphenyl)ethyl]-2- phenyl-4-(4-propylphenyl)- 1H-imidazole-5-carboxamide | 1.23 | 467.3 | 468.3 |
| 371 | 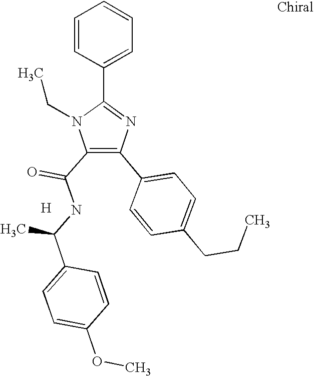 | 1-ethyl-N-[(1R)-1-(4- methoxyphenyl)ethyl]-2- phenyl-4-(4-propylphenyl)- 1H-imidazole-5-carboxamide | 1.23 | 467.3 | 468.3 |
| 372 | 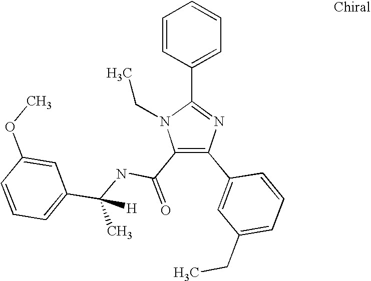 | 1-ethyl-4-(3-ethylphenyl)-N- [(1R)-1-(3- methoxyphenyl)ethyl]-2- phenyl-1H-imidazole-5- carboxamide | 1.2 | 453.2 | 454.3 |
| 373 | 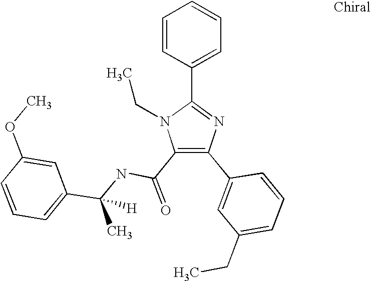 | 1-ethyl-4-(3-ethylphenyl)-N- [(1S)-1-(3- methoxyphenyl)ethyl]-2- phenyl-1H-imidazole-5- carboxamide | 1.2 | 453.2 | 454.3 |
| 374 | 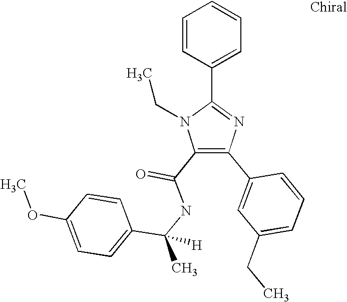 | 1-ethyl-4-(3-ethylphenyl)-N- [(1S)-1-(4- methoxyphenyl)ethyl]-2- phenyl-1H-imidazole-5- carboxamide | 1.2 | 453.2 | 454.3 |
| 375 | 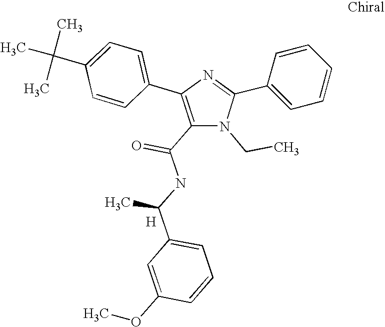 | 4-(4-tert-butylphenyl)-1-ethyl- N-[(1R)-1-(3- methoxyphenyl)ethyl]-2- phenyl-1H-imidazole-5- carboxamide | 1.24 | 481.3 | 482.3 |
| 376 | 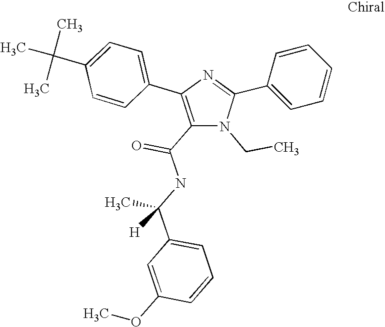 | 4-(4-tert-butylphenyl)-1-ethyl- N-[(1S)-1-(3- methoxyphenyl)ethyl]-2- phenyl-1H-imidazole-5- carboxamide | 1.24 | 481.3 | 482.3 |
| 377 | 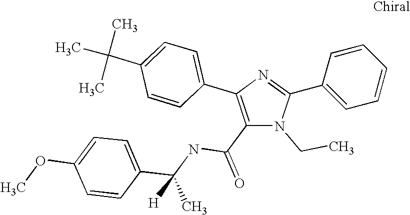 | 4-(4-tert-butylphenyl)-1-ethyl- N-[(1R)-1-(4- methoxyphenyl)ethyl]-2- phenyl-1H-imidazole-5- carboxamide | 1.23 | 481.3 | 482.3 |
| 378 | 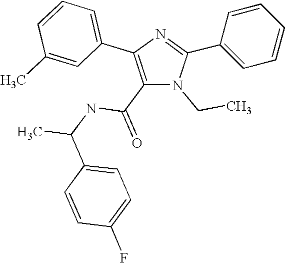 | 1-ethyl-N-[1-(4- fluorophenyl)ethyl]-4-(3- methylphenyl)-2-phenyl-1H- imidazole-5-carboxamide | 1.18 | 427.2 | 428.3 |
| 379 | 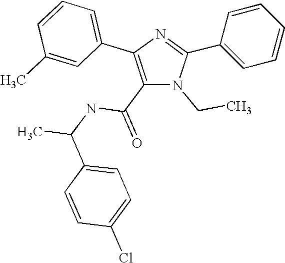 | N-[1-(4-chlorophenyl)ethyl]- 1-ethyl-4-(3-methylphenyl)-2- phenyl-1H-imidazole-5- carboxamide | 1.22 | 443.2 | 444.2 |
| 380 | 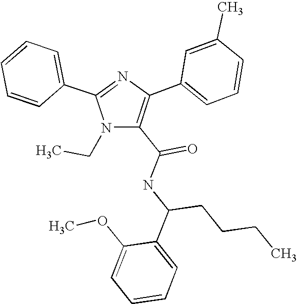 | 1-ethyl-N-[1-(2- methoxyphenyl)pentyl]-4-(3- methylphenyl)-2-phenyl-1H- imidazole-5-carboxamide | 1.26 | 481.3 | 482.3 |
| 381 | 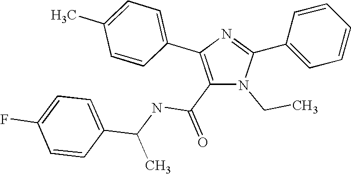 | 1-ethyl-N-[1-(4- fluorophenyl)ethyl]-4-(4- methylphenyl)-2-phenyl-1H- imidazole-5-carboxamide | 1.19 | 427.2 | 428.3 |
| 382 | 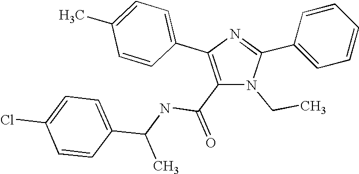 | N-[1-(4-chlorophenyl)ethyl]- 1-ethyl-4-(4-methylphenyl)-2- phenyl-1H-imidazole-5- carboxamide | 1.22 | 443.2 | 444.2 |
| 383 | 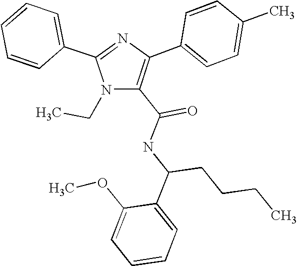 | 1-ethyl-N-[1-(2- methoxyphenyl)pentyl]-4-(4- methylphenyl)-2-phenyl-1H- imidazole-5-carboxamide | 1.26 | 481.3 | 482.3 |
| 384 | 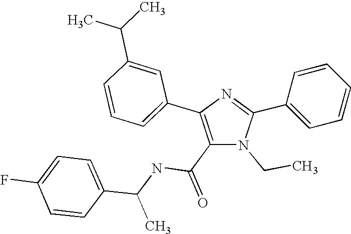 | 1-ethyl-N-[1-(4- fluorophenyl)ethyl]-4-(3- isopropylphenyl)-2-phenyl- 1H-imidazole-5-carboxamide | 1.23 | 455.2 | 456.3 |
| 385 | 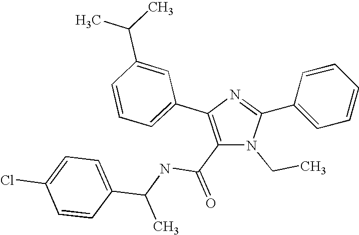 | N-[1-(4-chlorophenyl)ethyl]- 1-ethyl-4-(3-isopropylphenyl)- 2-phenyl-1H-imidazole-5- carboxamide | 1.25 | 471.2 | 472.3 |
| 386 | 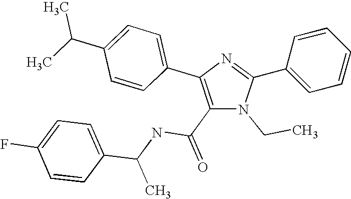 | 1-ethyl-N-[1-(4- fluorophenyl)ethyl]-4-(4- isopropylphenyl)-2-phenyl- 1H-imidazole-5-carboxamide | 1.23 | 455.2 | 456.3 |
| 387 | 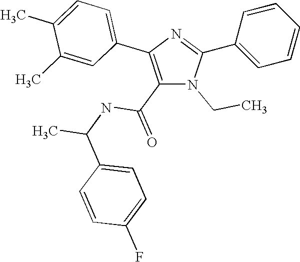 | 4-(3,4-dimethylphenyl)-1- ethyl-N-[1-(4- fluorophenyl)ethyl]-2-phenyl- 1H-imidazole-5-carboxamide | 1.19 | 441.2 | 442.3 |
| 388 | 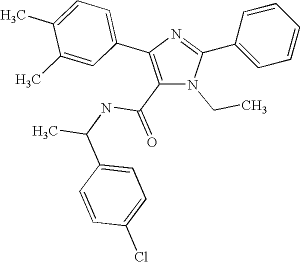 | N-[1-(4-chlorophenyl)ethyl]- 4-(3,4-dimethylphenyl)-1- ethyl-2-phenyl-1H-imidazole- 5-carboxamide | 1.23 | 457.2 | 458.3 |
| 389 | 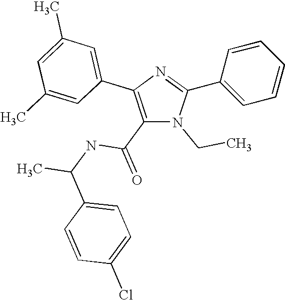 | N-[1-(4-chlorophenyl)ethyl]- 4-(3,5-dimethylphenyl)-1- ethyl-2-phenyl-1H-imidazole- 5-carboxamide | 1.23 | 457.2 | 458.3 |
| 390 | 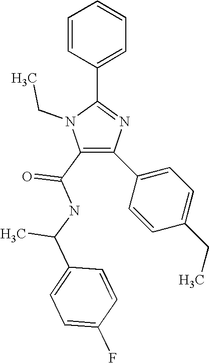 | 1-ethyl-4-(4-ethylphenyl)-N- [1-(4-fluorophenyl)ethyl]-2- phenyl-1H-imidazole-5- carboxamide | 1.2 | 441.2 | 442.3 |
| 391 | 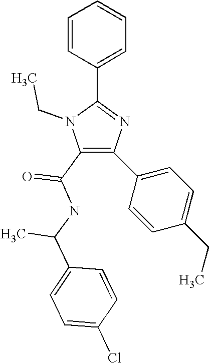 | N-[1-(4-chlorophenyl)ethyl]- 1-ethyl-4-(4-ethylphenyl)-2- phenyl-1H-imidazole-5- carboxamide | 1.24 | 457.2 | 458.3 |
| 392 | 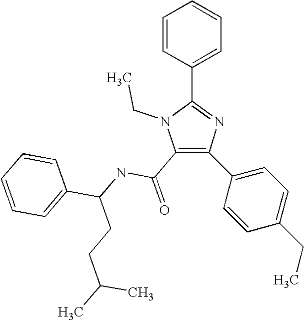 | 1-ethyl-4-(4-ethylphenyl)-N- (4-methyl-1-phenylpentyl)-2- phenyl-1H-imidazole-5- carboxamide | 1.29 | 479.3 | 480.4 |
| 393 | 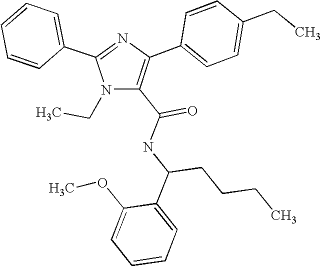 | 1-ethyl-4-(4-ethylphenyl)-N- [1-(2-methoxyphenyl)pentyl]- 2-phenyl-1H-imidazole-5- carboxamide | 1.27 | 495.3 | 496.4 |
| 394 | 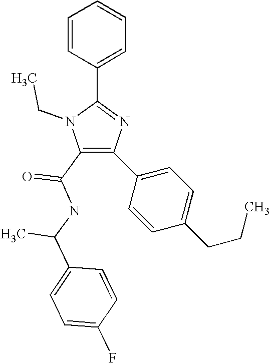 | 1-ethyl-N-[1-(4- fluorophenyl)ethyl]-2-phenyl- 4-(4-propylphenyl)-1H- imidazole-5-carboxamide | 1.24 | 455.2 | 456.3 |
| 395 | 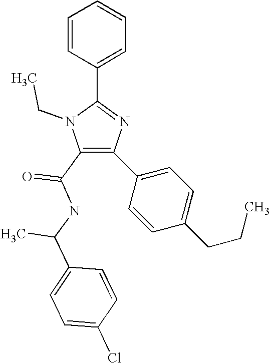 | N-[1-(4-chlorophenyl)ethyl]- 1-ethyl-2-phenyl-4-(4- propylphenyl)-1H-imidazole- 5-carboxamide | 1.26 | 471.2 | 472.3 |
| 396 | 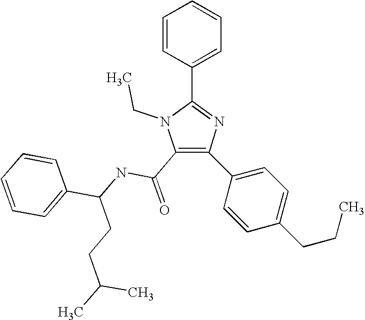 | 1-ethyl-N-(4-methyl-1- phenylpentyl)-2-phenyl-4-(4- propylphenyl)-1H-imidazole- 5-carboxamide | 1.32 | 493.3 | 494.4 |
| 397 | 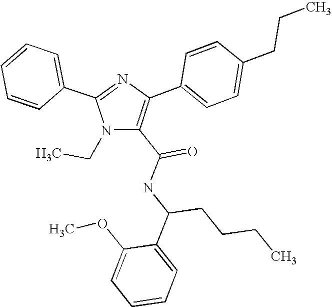 | 1-ethyl-N-[1-(2- methoxyphenyl)pentyl]-2- phenyl-4-(4-propylphenyl)- 1H-imidazole-5-carboxamide | 1.3 | 509.3 | 510.4 |
| 398 | 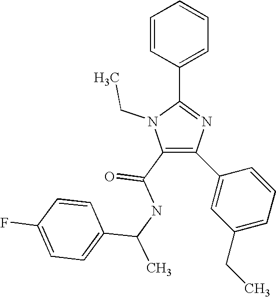 | 1-ethyl-4-(3-ethylphenyl)-N- [1-(4-fluorophenyl)ethyl]-2- phenyl-1H-imidazole-5- carboxamide | 1.21 | 441.2 | 442.3 |
| 399 | 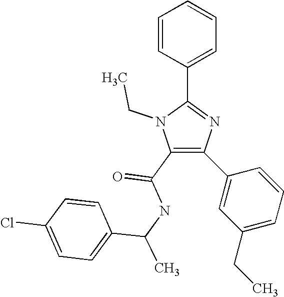 | N-[1-(4-chlorophenyl)ethyl]- 1-ethyl-4-(3-ethylphenyl)-2- phenyl-1H-imidazole-5- carboxamide | 1.23 | 457.2 | 458.3 |
| 400 | 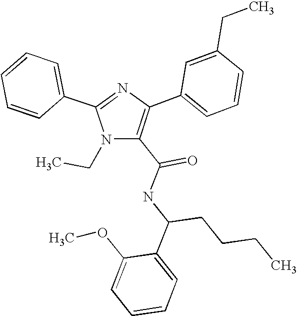 | 1-ethyl-4-(3-ethylphenyl)-N- [1-(2-methoxyphenyl)pentyl]- 2-phenyl-1H-imidazole-5- carboxamide | 1.28 | 495.3 | 496.4 |
| 401 | 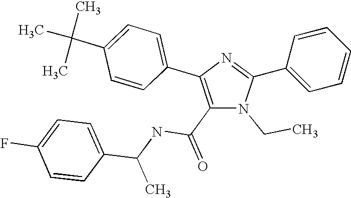 | 4-(4-tert-butylphenyl)-1-ethyl- N-[1-(4-fluorophenyl)ethyl]- 2-phenyl-1H-imidazole-5- carboxamide | 1.24 | 469.3 | 470.3 |
| 402 | 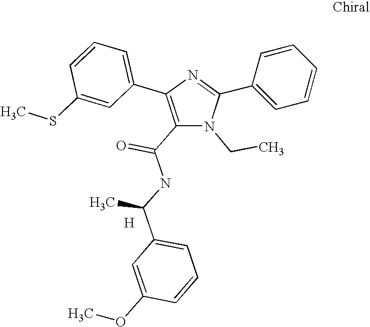 | 1-ethyl-N-[(1R)-1-(3- methoxyphenyl)ethyl]-4-[3- (methylthio)phenyl]-2-phenyl- 1H-imidazole-5-carboxamide | 1.2 | 471.2 | 472.3 |
| 403 | 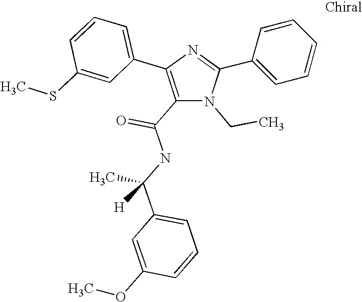 | 1-ethyl-N-[(1S)-1-(3- methoxyphenyl)ethyl]-4-[3- (methylthio)phenyl]-2-phenyl- 1H-imidazole-5-carboxamide | 1.22 | 471.2 | 472.3 |
| 404 | 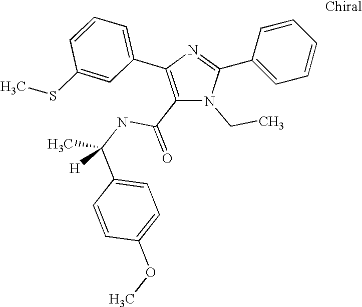 | 1-ethyl-N-[(1S)-1-(4- methoxyphenyl)ethyl]-4-[3- (methylthio)phenyl]-2-phenyl- 1H-imidazole-5-carboxamide | 1.19 | 471.2 | 472.3 |
| 405 | 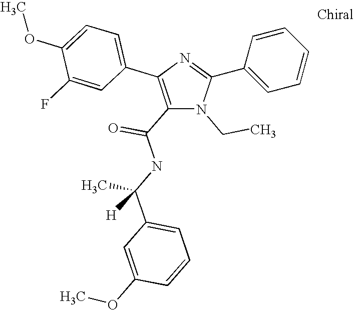 | 1-ethyl-4-(3-fluoro-4- methoxyphenyl)-N-[(1S)-1-(3- methoxyphenyl)ethyl]-2- phenyl-1H-imidazole-5- carboxamide | 1.17 | 473.2 | 474.3 |
| 406 | 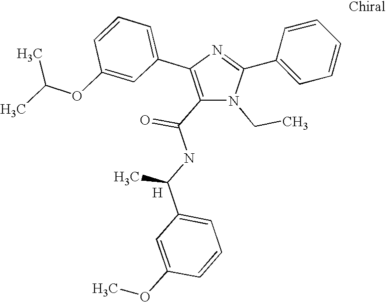 | 1-ethyl-4-(3- isopropoxyphenyl)-N-[(1R)-1- (3-methoxyphenyl)ethyl]-2- phenyl-1H-imidazole-5- carboxamide | 1.2 | 483.3 | 484.3 |
| 407 | 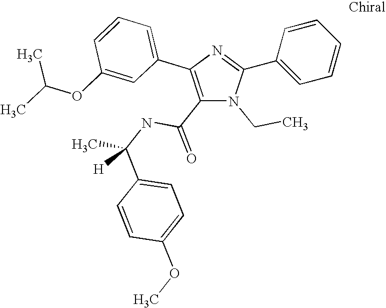 | 1-ethyl-4-(3- isopropoxyphenyl)-N-[(1S)-1- (4-methoxyphenyl)ethyl]-2- phenyl-1H-imidazole-5- carboxamide | 1.2 | 483.3 | 484.3 |
| 408 | 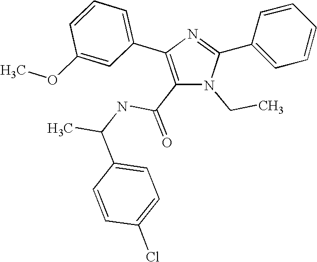 | N-[1-(4-chlorophenyl)ethyl]- 1-ethyl-4-(3-methoxyphenyl)- 2-phenyl-1H-imidazole-5- carboxamide | 1.2 | 459.2 | 460.2 |
| 409 | 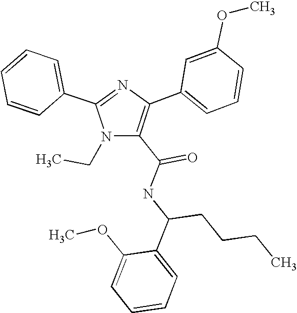 | 1-ethyl-4-(3-methoxyphenyl)- N-[1-(2-methoxyphenyl)pentyl]- 2-phenyl-1H-imidazole-5- carboxamide | 1.25 | 497.3 | 498.3 |
| 410 | 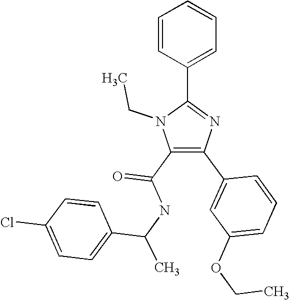 | N-[1-(4-chlorophenyl)ethyl]- 4-(3-ethoxyphenyl)-1-ethyl-2- phenyl-1H-imidazole-5- carboxamide | 1.23 | 473.2 | 474.2 |
| 411 | 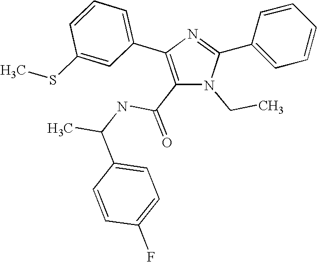 | 1-ethyl-N-[1-(4- fluorophenyl)ethyl]-4-[3- (methylthio)phenyl]-2-phenyl- 1H-imidazole-5-carboxamide | 1.2 | 459.2 | 460.2 |
| 412 | 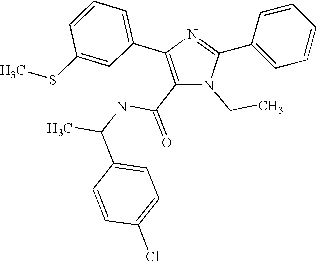 | N-[1-(4-chlorophenyl)ethyl]- 1-ethyl-4-[3- (methylthio)phenyl]-2-phenyl- 1H-imidazole-5-carboxamide | 1.23 | 475.1 | 476.2 |
| 413 | 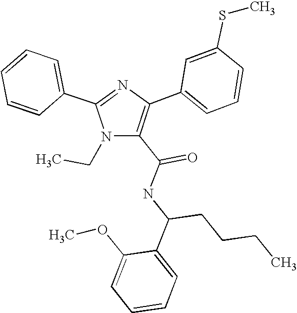 | 1-ethyl-N-[1-(2- methoxyphenyl)pentyl]-4-[3- (methylthio)phenyl]-2-phenyl- 1H-imidazole-5-carboxamide | 1.27 | 513.2 | 514.3 |
| 414 | 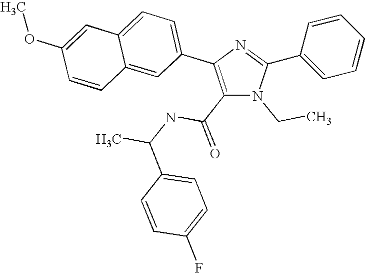 | 1-ethyl-N-[1-(4- fluorophenyl)ethyl]-4-(6- methoxy-2-naphthyl)-2- phenyl-1H-imidazole-5- carboxamide | 1.21 | 493.2 | 494.3 |
| 415 | 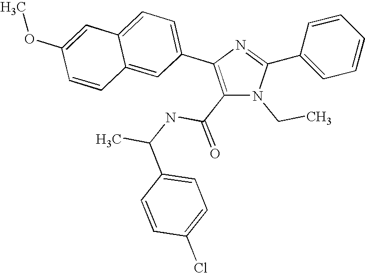 | N-[1-(4-chlorophenyl)ethyl]- 1-ethyl-4-(6-methoxy-2- naphthyl)-2-phenyl-1H- imidazole-5-carboxamide | 1.23 | 509.2 | 510.3 |
| 416 | 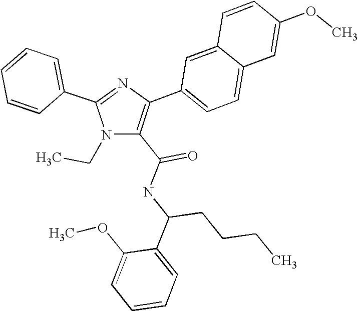 | 1-ethyl-4-(6-methoxy-2- naphthyl)-N-[1-(2- naphthyl)-N-[1-(2- methoxyphenyl)pentyl]-2- phenyl-1H-imidazole-5- carboxamide | 1.27 | 547.3 | 548.3 |
| 417 | 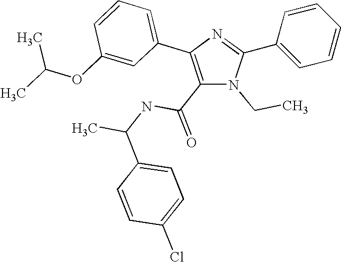 | N-[1-(4-chlorophenyl)ethyl]- 1-ethyl-4-(3- isopropoxyphenyl)-2-phenyl- 1H-imidazole-5-carboxamide | 1.24 | 487.2 | 488.3 |
| 418 | 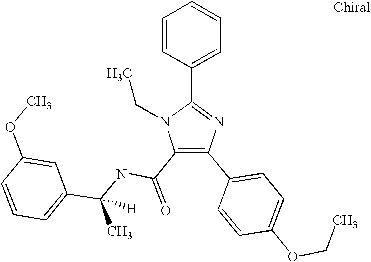 | 4-(4-ethoxyphenyl)-1-ethyl-N- [(1S)-1-(3- methoxyphenyl)ethyl]-2- phenyl-1H-imidazole-5- carboxamide | 1.18 | 469.2 | 470.3 |
| 419 | 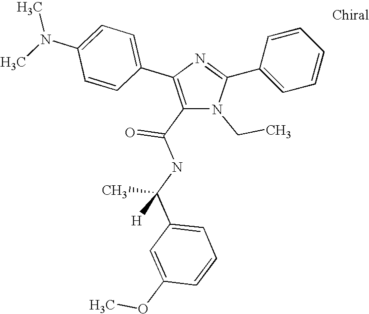 | 4-[4-(dimethylamino)phenyl]- 1-ethyl-N-[(1S)-1-(3- methoxyphenyl)ethyl]-2- phenyl-1H-imidazole-5- carboxamide | 1.17 | 468.3 | 469.3 |
| 420 | 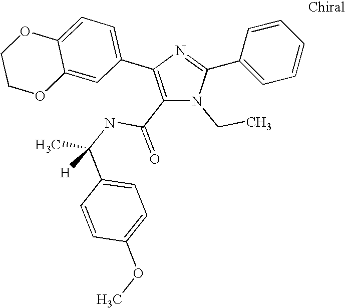 | 4-(2,3-dihydro-1,4- benzodioxin-6-yl)-1-ethyl-N- [(1S)-1-(4- methoxyphenyl)ethyl]-2- phenyl-1H-imidazole-5- carboxamide | 1.15 | 483.2 | 484.3 |
| 421 | 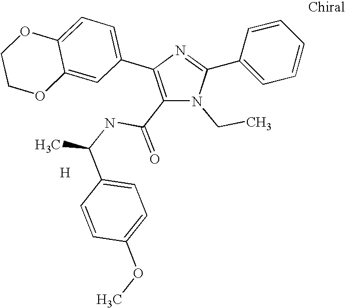 | 4-(2,3-dihydro-1,4- benzodioxin-6-yl)-1-ethyl-N- [(1R)-1-(4- methoxyphenyl)ethyl]-2- phenyl-1H-imidazole-5- carboxamide | 1.15 | 483.2 | 484.3 |
| 422 | 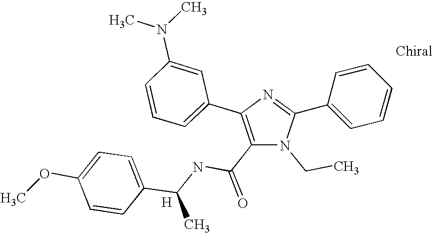 | 4-[3-(dimethylamino)phenyl]- 1-ethyl-N-[(1S)-1-(4- methoxyphenyl)ethyl]-2- phenyl-1H-imidazole-5- carboxamide | 1.16 | 468.3 | 469.3 |
| 423 | 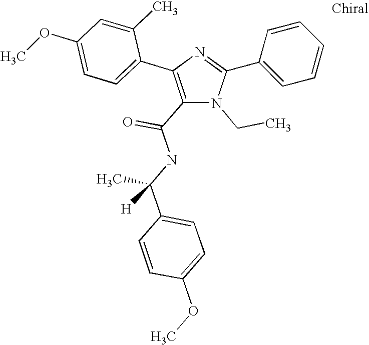 | 1-ethyl-4-(4-methoxy-2- methylphenyl)-N-[(1S)-1-(4- methoxyphenyl)ethyl]-2- phenyl-1H-imidazole-5- carboxamide | 1.17 | 469.2 | 470.3 |
| 424 | 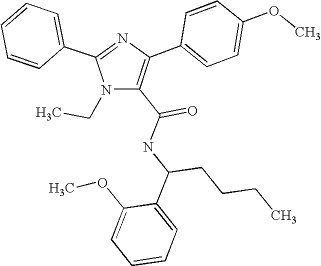 | 1-ethyl-4-(4-methoxyphenyl)- N-[1-(2- methoxyphenyl)pentyl]-2- phenyl-1H-imidazole-5- carboxamide | 1.23 | 497.3 | 498.3 |
| 425 | 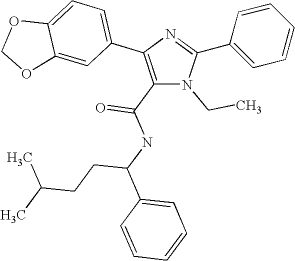 | 4-(1,3-benzodioxol-5-yl)-1- ethyl-N-(4-methyl-1- phenylpentyl)-2-phenyl-1H- imidazole-5-carboxamide | 1.24 | 495.3 | 496.3 |
| 426 | 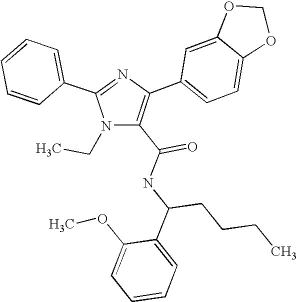 | 4-(1,3-benzodioxol-5-yl)-1- ethyl-N-[1-(2- methoxyphenyl)pentyl]-2- phenyl-1H-imidazole-5- carboxamide | 1.23 | 511.2 | 512.3 |
| 427 | 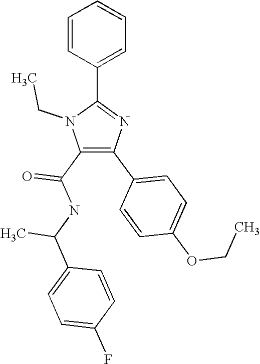 | 4-(4-ethoxyphenyl)-1-ethyl-N- [1-(4-fluorophenyl)ethyl]-2- phenyl-1H-imidazole-5- carboxamide | 1.19 | 457.2 | 458.3 |
| 428 | 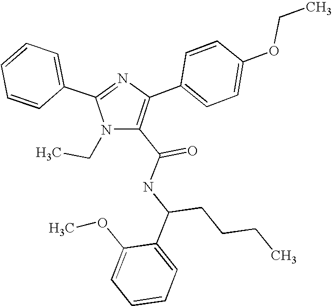 | 4-(4-ethoxyphenyl)-1-ethyl-N- [1-(2-methoxyphenyl)pentyl]- 2-phenyl-1H-imidazole-5- carboxamide | 1.26 | 511.3 | 512.3 |
| 429 | 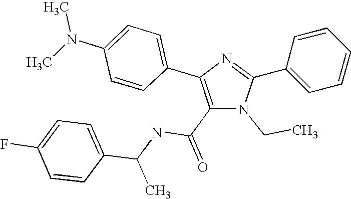 | 4-[4-(dimethylamino)phenyl]- 1-ethyl-N-[1-(4- fluorophenyl)ethyl]-2-phenyl- 1H-imidazole-5-carboxamide | 1.17 | 456.2 | 457.3 |
| 430 | 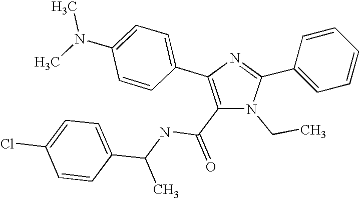 | N-[1-(4-chlorophenyl)ethyl]- 4-[4-(dimethylamino)phenyl]- 1-ethyl-2-phenyl-1H- imidazole-5-carboxamide | 1.2 | 472.2 | 473.3 |
| 431 | 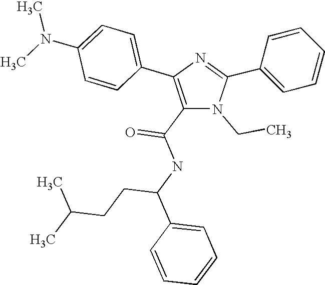 | 4-[4-(dimethylamino)phenyl]- 1-ethyl-N-(4-methyl-1- phenylpentyl)-2-phenyl-1H- imidazole-5-carboxamide | 1.26 | 494.3 | 495.4 |
| 432 | 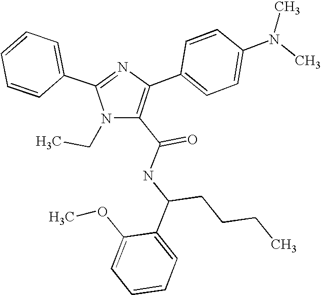 | 4-[4-(dimethylamino)phenyl]- 1-ethyl-N-[1-(2- methoxyphenyl)pentyl]-2- phenyl-1H-imidazole-5- carboxamide | 1.24 | 510.3 | 511.4 |
| 433 | 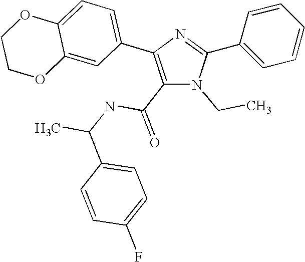 | 4-(2,3-dihydro-1,4- benzodioxin-6-yl)-1-ethyl-N- [1-(4-fluorophenyl)ethyl]-2- phenyl-1H-imidazole-5- carboxamide | 1.16 | 471.2 | 472.3 |
| 434 | 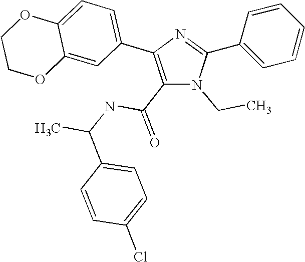 | N-[1-(4-chlorophenyl)ethyl]- 4-(2,3-dihydro-1,4- benzodioxin-6-yl)-1-ethyl-2- phenyl-1H-imidazole-5- carboxamide | 1.19 | 487.2 | 488.2 |
| 435 | 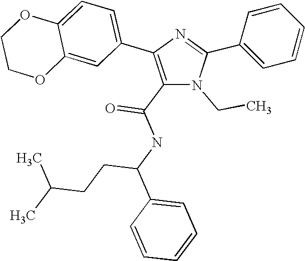 | 4-(2,3-dihydro-1,4- benzodioxin-6-yl)-1-ethyl-N- (4-methyl-1-phenylpentyl)-2- phenyl-1H-imidazole-5- carboxamide | 1.24 | 509.3 | 510.3 |
| 436 | 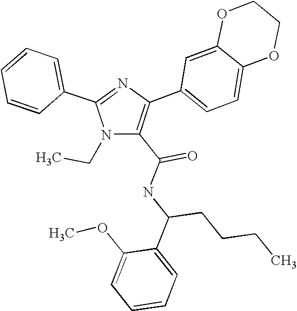 | 4-(2,3-dihydro-1,4- benzodioxin-6-yl)-1-ethyl-N- [1-(2-methoxyphenyl)pentyl]- 2-phenyl-1H-imidazole-5- carboxamide | 1.23 | 525.3 | 526.3 |
| 437 | 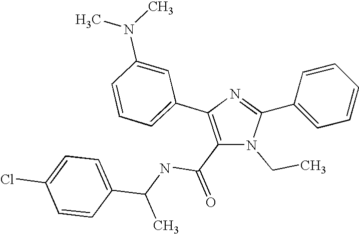 | N-[1-(4-chlorophenyl)ethyl]- 4-[3-(dimethylamino)phenyl]- 1-ethyl-2-phenyl-1H- imidazole-5-carboxamide | 1.2 | 472.2 | 473.3 |
| 438 | 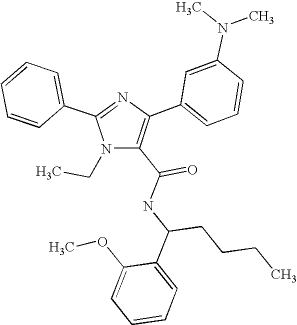 | 4-[3-(dimethylamino)phenyl]- 1-ethyl-N-[1-(2- methoxyphenyl)pentyl]-2- phenyl-1H-imidazole-5- carboxamide | 1.25 | 510.3 | 511.4 |
| 439 | 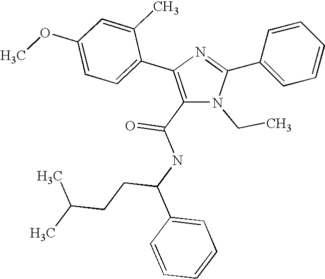 | 1-ethyl-4-(4-methoxy-2- methylphenyl)-N-(4-methyl-1- phenylpentyl)-2-phenyl-1H- imidazole-5-carboxamide | 1.27 | 495.3 | 496.4 |
| 440 | 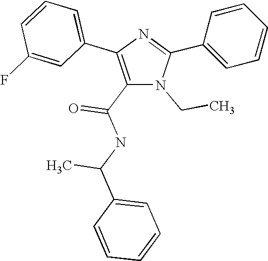 | 1-ethyl-4-(3-fluorophenyl)-2- phenyl-N-(1-phenylethyl)-1H- imidazole-5-carboxamide | 1.26 | 413.2 | 414.1 |
| 441 | 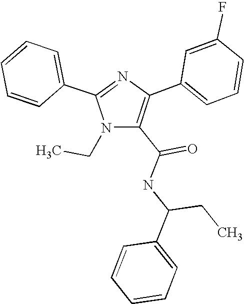 | 1-ethyl-4-(3-fluorophenyl)-2- phenyl-N-(1-phenylpropyl)- 1H-imidazole-5-carboxamide | 1.28 | 427.2 | 428.1 |
| 442 | 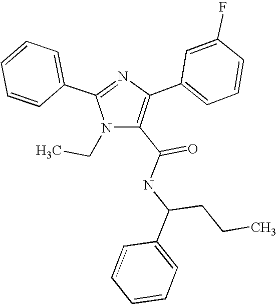 | 1-ethyl-4-(3-fluorophenyl)-2- phenyl-N-(1-phenylbutyl)-1H- imidazole-5-carboxamide | 1.31 | 441.2 | 442.2 |
| 443 | 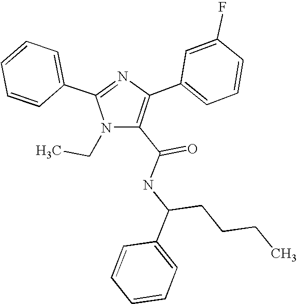 | 1-ethyl-4-(3-fluorophenyl)-2- phenyl-N-(1-phenylpentyl)- 1H-imidazole-5-carboxamide | 1.34 | 455.2 | 456.2 |
| 444 | 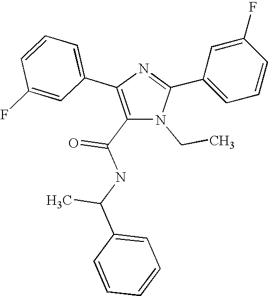 | 1-ethyl-2,4-bis(3- fluorophenyl)-N-(1- phenylethyl)-1H-imidazole-5- carboxamide | 1.29 | 431.2 | 432.1 |
| 445 | 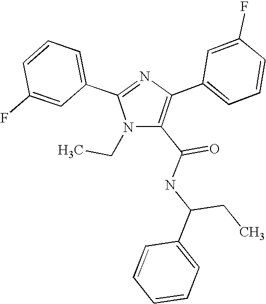 | 1-ethyl-2,4-bis(3- fluorophenyl)-N-(1- phenylpropyl)-1H-imidazole- 5-carboxamide | 1.32 | 445.2 | 446.1 |
| 446 | 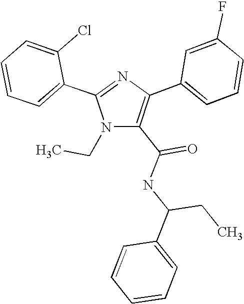 | 2-(2-chlorophenyl)-1-ethyl-4- (3-fluorophenyl)-N-(1- phenylpropyl)-1H-imidazole- 5-carboxamide | 1.33 | 461.2 | 462.1 |

### Example 4

### Assay for NL-3 Receptor Binding Activity

The following assay is a standard assay for NK-3 receptor binding activity. Assays are performed as described in Krause et al (Proc. Natl. Acad. Sci. USA 94:310-15, 1997). The NK-3 receptor complementary DNA is cloned from human hypothalamic RNA using standard procedures. The receptor cDNA is inserted into the expression vector pM² to transfect the mammalian Chinese hamster ovary cell line, and a stably expressing clonal cell line is isolated, characterized and used for the current experiments. Cells are grown in minimal essential medium alpha containing 10% fetal bovine serum and 250 μg/ml G418. Cells are liberated from cell culture plates with No-zyme (PBS base, JRH Biosciences), and harvested by low speed centrifugation. The cell pellet is homogenized in TBS (0.05 m TrisHCl, 120 mM NaCl, pH 7.4) with a Polytron homogenizer at setting 5 for 20 seconds, and total cellular membranes are isolated by centrifugation at 47,500×g for 10 minutes. The membrane pellet is resuspended by homogenization with the Polytron as above, and the membranes are isolated by centrifugation at 47,500×g for 10 minutes. This final membrane pellet is resuspended in TBS at a protein concentration of 350 μg/ml.

Receptor binding assays contain a total volume of 200 μl containing 50 μg membrane protein, 0.05-0.15 nM ¹²⁵I-methylPhe⁷-neurokinin B, drug or blocker in TBS containing 1.0 mg/ml bovine serum albumen, 0.2 mg/ml bacitracin, 20 μg/ml leupeptin and 20 μg/ml chymostatin. Incubations are carried out for 2 hours at 4° C., and the membrane proteins are harvested by passing the incubation mixture by rapid filtration over presoaked GF/B filters to separate bound from free ligand. The filters are presoaked in TBS containing 2% BSA and 0.1% Tween 20. After filtration of the incubation mixture, filters are rinsed 4 times with ice-cold TBS containing 0.01% sodium dodecyl sulfate and radioactivity is quantitated in a β-plate scintillation counter. One μM methylPhe⁷-neurokinin B is added to some tubes to determine nonspecific binding. Data are collected in duplicate determinations, averaged, and the percent inhibition of total specific binding is calculated. The total specific binding is the total binding minus the nonspecific binding. In many cases, the concentration of unlabeled drug is varied and total displacement curves of binding is carried out. Data are converted to a form for the calculation of IC₅₀ and Hill coefficient (nH).

### Example 5

### Assay for NK-3 Functional Activity

This Example illustrates a calcium mobilization assays for evaluating NK-3 receptor modulator activity.

The human NK-3 receptor-bearing Chinese hamster ovary cells are grown in minimal essential media supplemented with 250 μg/ml G418, 10% fetal bovine serum and 25 mM HEPES, pH=7.4. Forty eight hours prior to the day of assay, the cells are plated in fresh media that does not contain the G418. On the day of assay, cells grown to 70-90% confluency in 96-well plates are washed with Krebs-Ringer buffer (25 mM HEPES, 5 mM KCl, 0.96 mM NaH₂PO₄, 1 mM MgSO₄, 2 mM CaCl₂, 5 mM glucose, 1 mM probenecid, pH 7.4) and are then incubated for 1-2 hours in the above buffer supplemented with Fluo3-AM (2.5 to 10 μg/ml; Teflabs) at 37 degrees C. in an environment containing 5% CO₂. The wells are then washed twice with Krebs Ringers HEPES buffer. Agonist-induced (methylPhe7-neurokinin B) calcium mobilization is monitored using a FLIPR (Molecular Devices) instrument. The agonist is added to the cells and fluorescence responses are continuously recorded for up to 5 minutes. For the examination of antagonist drug candidates, compounds are preincubated with the cells for up to 30 min. prior to administration of the methylPhe7-neurokinin B agonist usually at a concentration that brings about a 50% maximal activity. Responses are recorded for times up to 5 min. Kaleidagraph software (Synergy Software, Reading, Pa.) is utilized to fit the data to the equation y=a*(1/(1+(b/x)c)) to determine the EC₅₀ value or IC₅₀ value for the response. In this equation, y is the maximum fluorescence signal; x is the concentration of the agonist or antagonist; a is the Eₘₐₓ; b corresponds to the EC₅₀ or IC₅₀ value; and c is the Hill coefficient.

### Example 6

### Microsomal In Vitro Half-Life

This Example illustrates the evaluation of compound half-life values (t_{1/2} values) using a representative liver microsomal half-life assay.

Pooled human liver microsomes are obtained from XenoTech LLC (Kansas City, Kans.). Such liver microsomes may also be obtained from In Vitro Technologies (Baltimore, Md.) or Tissue Transformation Technologies (Edison, N.J.). Six test reactions are prepared, each containing 25 μl microsomes, 5 μl of a 100 μM solution of test compound, and 399 μl 0.1 M phosphate buffer (19 mL 0.1 M NaH₂PO₄, 81 mL 0.1 M Na₂HPO₄, adjusted to pH 7.4 with H₃PO₄). A seventh reaction is prepared as a positive control containing 25 μl microsomes, 399 μl 0.1 M phosphate buffer, and 5 μl of a 100 μM solution of a compound with known metabolic properties (e.g., DIAZEPAM or CLOZAPINE). Reactions are preincubated at 39° C. for 10 minutes.

CoFactor Mixture is prepared by diluting 16.2 mg NADP and 45.4 mg Glucose-6-phosphate in 4 mL 100 mM MgCl₂. Glucose-6-phosphate dehydrogenase solution is prepared by diluting 214.3 μl glucose-6-phosphate dehydrogenase suspension (Roche Molecular Biochemicals; Indianapolis, Ind.) into 1285.7 μl distilled water. 71 μl Starting Reaction Mixture (3 mL CoFactor Mixture; 1.2 mL Glucose-6-phosphate dehydrogenase solution) is added to 5 of the 6 test reactions and to the positive control. 71 μl 100 mM MgCl₂ is added to the sixth test reaction, which is used as a negative control. At each time point (0, 1, 3, 5, and 10 minutes), 75 μl of each reaction mix is pipetted into a well of a 96-well deep-well plate containing 75 μl ice-cold acetonitrile. Samples are vortexed and centrifuged 10 minutes at 3500 rpm (Sorval T 6000D centrifuge, H1000B rotor). 75 μl of supernatant from each reaction is transferred to a well of a 96-well plate containing 150 μl of a 0.5 μM solution of a compound with a known LCMS profile (internal standard) per well. LCMS analysis of each sample is carried out and the amount of unmetabolized test compound is measured as AUC, compound concentration vs. time is plotted, and the t_{1/2} value of the test compound is extrapolated.

Preferred compounds provided herein exhibit in vitro t_{1/2} values of greater than 10 minutes and less than 4 hours, preferably between 30 minutes and 1 hour, in human liver microsomes.

### Example 7

### MDCK Toxicity Assay

This Example illustrates the evaluation of compound toxicity using a Madin Darby canine kidney (MDCK) cell cytotoxicity assay.

1 μL of test compound is added to each well of a clear bottom 96-well plate (PACKARD, Meriden, Conn.) to give final concentration of compound in the assay of 10 micromolar, 100 micromolar or 200 micromolar. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested, and diluted to a concentration of 0.1×10⁶ cells/ml with warm (37° C.) medium (VITACELL Minimum Essential Medium Eagle, ATCC catalog # 30-2003). 100 μL of diluted cells is added to each well, except for five standard curve control wells that contain 100 μL of warm medium without cells. The plate is then incubated at 37° C. under 95% O₂, 5% CO₂ for 2 hours with constant shaking. After incubation, 50 μL of mammalian cell lysis solution (from the PACKARD (Meriden, Conn.) ATP-LITE-M Luminescent ATP detection kit) is added per well, the wells are covered with PACKARD TOPSEAL stickers, and plates are shaken at approximately 700 rpm on a suitable shaker for 2 minutes.

Compounds causing toxicity will decrease ATP production, relative to untreated cells. The ATP-LITE-M Luminescent ATP detection kit is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 mL of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 100 nM, 50 nM, 25 nM and 12.5 nM. PACKARD substrate solution (50 μL) is added to all wells, which are then covered, and the plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

1. A compound of the formula: 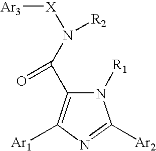
or a pharmaceutically acceptable salt thereof, wherein:
X is N(R₃ₐ) or C(R₃ₐ)(R_{3b});
Ar₁ is a 6- to 10-membered aryl, a 5- to 10-membered heteroaryl, or a group of the formula 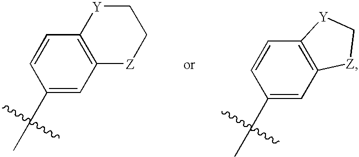
wherein Y and Z are independently CH₂, NH, O or S;
each of which is substituted with from 0 to 4 substituents independently chosen from halogen, cyano, nitro, oxo and groups of the formula L-M;
Ar₂ is a 6- to 10-membered aryl or 5- to 10-membered heteroaryl, each of which is substituted with from 0 to 4 substituents independently chosen from halogen, cyano, nitro, oxo and groups of the formula L-M;
Ar₃ is a 6- to 10-membered aryl or a 6- to 10-membered heteroaryl, each of which is substituted with from 0 to 4 substituents independently chosen from:
(i) halogen, cyano, nitro and oxo;
(ii) groups of the formula L-M; and
(iii) groups that are taken together with R₃ₐ or R_{3b} to form a fused, partially saturated 5- to 8-membered carbocycle or heterocycle;
R₁ is C₁-C₆alkyl substituted with from 0 to 6 substituents independently chosen from halogen, cyano, nitro, oxo and groups of the formula L-M;
R₂ is:
(i) hydrogen, C₁-C₆alkyl or C₁-C₆alkenyl, each of which alkyl and alkenyl is substituted with from 0 to 6 substituents independently chosen from R_{b}; or
(ii) taken together with R₃ₐ or R_{3b} to form a 4- to 8-membered heterocycloalkyl substituted with from 0 to 4 substituents independently chosen from halogen, cyano, nitro, oxo and groups of the formula L-M;
R₃ₐ and R_{3b} are:
(i) independently
(a) hydrogen, C₁-C₈alkyl, C₁-C₈alkenyl, C₃-C₈cycloalkyl or phenyl, wherein each alkyl, alkenyl, cycloalkyl and phenyl is substituted with from 0 to 6 substituents independently chosen from halogen, cyano, nitro, oxo and groups of the formula L-M;
(b) taken together with R₂ to form an optionally substituted 4- to 8-membered heterocycloalkyl; or
(c) taken together with a substituent of Ar₃ to form a fused, partially saturated, 5- to 8-membered carbocycle or heterocycle, each of which is substituted with from 0 to 4 substituents independently chosen from halogen, cyano, nitro, oxo and groups of the formula L-M;
such that at least one of R₃ₐ and R_{3b} is not hydrogen; or
(ii) taken together to form a spiro 3- to 8-membered carbocycle or heterocycle;
L is independently selected at each occurrence from a bond, O, C(═O), OC(═O), C(═O)O, OC(═O)O, S(O)ₘ, N(Rₓ), C(═O)N(Rₓ), N(Rₓ)C(═O), N(Rₓ)S(O)ₘ, S(O)ₘN(Rₓ) and N[S(O)ₘRₓ]S(O)ₘ; wherein m is independently selected at each occurrence from 0, 1 and 2; and Rₓ is independently selected at each occurrence from hydrogen and C₁-C₈alkyl;
M is independently selected at each occurrence from: (i) hydrogen; and (ii) C₁-C₆alkyl, C₂-C₆alkenyl, C₂-C₆alkynyl, phenylC₀-C₆alkyl and (3- to 10-membered heterocycle)C₀-C₆alkyl, each of which is substituted with from 0 to 6 substituents independently chosen from R_{b}; and
R_{b} is independently chosen at each occurrence from:
(i) hydroxy, halogen, amino, aminocarbonyl, cyano, nitro, oxo and —COOH; and
(ii) C₁-C₆alkyl, C₁-C₆alkenyl, C₁-C₆alkynyl, haloC₁-C₆alkyl, hydroxyC₁-C₆alkyl, cyanoC₁-C₈alkyl, C₁-C₆alkoxy, haloC₁-C₈alkoxy, C₁-C₆alkanoyl, C₂-C₆alkoxycarbonyl, C₂-C₆alkanoyloxy, C₁-C₆alkylthio, C₂-C₆alkylether, 4- to 8- membered heterocycloalkyl, and mono- and di-(C₁-C₆alkyl)aminoC₀-C₆alkyl.

2. A compound according to claim 1, wherein X is N(R₃ₐ), and wherein R₃ₐ is hydrogen, methyl, ethyl or propyl.

3. A compound or salt according to claim 1, wherein the compound has the formula:

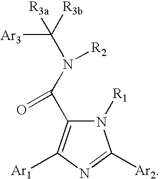

4. A compound or salt according to claim 3, wherein R₃ₐ is hydrogen and R_{3b} is C₁-C₆alkyl, C₁-C₆alkenyl, C₄-C₇cycloalkyl or phenylC₀-C₂alkyl, each of which is substituted with from 0 to 3 substituents independently chosen from halogen, hydroxy and cyano.

5. A compound or salt according to claim 4, wherein R_{3b} is C₁-C₄alkyl.

6. A compound or salt according to claim 3, wherein R₃ₐ is hydrogen and R_{3b} is taken together with a substituent of Ar₃ to form an optionally substituted, fused 6- or 7-membered carbocycle or heterocycle.

7. A compound or salt according to claim 6, wherein the group represented by 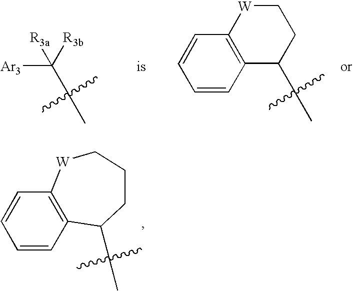
each of which is substituted with from 0 to 2 substituents independently chosen from methyl, ethyl and oxo, wherein W is CH₂, NH, O or S.

8. A compound or salt according to claim 1, wherein R₁ is hydrogen or C₁-C₄alkyl.

9. A compound or salt according to claim 8, wherein R₁ is methyl or ethyl.

10. A compound or salt according to claim 1, wherein R₂ is hydrogen or C₁-C₄alkyl.

11. A compound or salt according to claim 10, wherein R₂ is hydrogen.

12. A compound or salt according to claim 1, wherein Ar₁ is phenyl, thiazolyl, thiophenyl, furanyl, pyridyl, pyrimidyl, naphthyl, 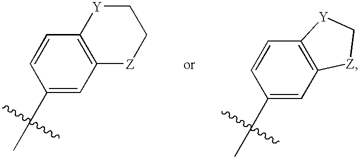
wherein Y and Z are independently CH₂, NH, O or S;
each of which is substituted with from 0 to 4 substituents independently chosen from:
(i) hydroxy, halogen, cyano, amino, C₁-C₆alkyl, C₁-C₆alkenyl, C₁-C₆alkynyl, haloC₁-C₆alkyl, hydroxyC₁-C₆alkyl, cyanoC₁-C₆alkyl, C₁-C₆alkoxy, C₁-C₆alkylthio, C₂-C₆alkyl ether, haloC₁-C₆alkoxy, and mono- and di-(C₁-C₆alkyl) aminoC₀-C₆alkyl; and
(ii) phenyl, benzyl and phenoxy, each of which is optionally substituted with halogen, C₁-C₄alkyl and C₁-C₄alkoxy.

13. A compound or salt according to claim 12, wherein Ar₁ is phenyl, thiazolyl or pyridyl, each of which is substituted with from 0 to 3 substituents independently chosen from halogen, C₁-C₄alkyl, haloC₁-C₄alkyl, C₁-C₄alkoxy, C₁-C₄alkylthio and haloC₁-C₄alkoxy.

14. A compound or salt according to claim 1, wherein Ar₂ is phenyl, thiazolyl, pyridyl or pyrimidyl, each of which is substituted with from 0 to 4 substituents independently chosen from hydroxy, halogen, cyano, amino, C₁-C₆alkyl, haloC₁-C₆alkyl, C₁-C₆alkoxy and haloC₁-C₆alkoxy.

15. A compound or salt according to claim 14, wherein Ar₂ is phenyl, thiazolyl or pyridyl, each of which is unsubstituted or substituted with a halogen.

16. A compound or salt according to claim 1, wherein Ar₃ is phenyl, naphthyl or pyridyl, each of which is substituted with from 0 to 4 substituents independently chosen from hydroxy, halogen, cyano, amino, C₁-C₆alkyl, haloC₁-C₆alkyl, hydroxyC₁-C₆alkyl, cyanoC₁-C₆alkyl, C₁-C₆alkoxy and haloC₁-C₆alkoxy.

17. A compound or salt according to claim 16, wherein Ar₃ is phenyl that is unsubstituted or substituted with halogen, C₁-C₄alkyl or C₁-C₄alkoxy.

18. A compound or salt according to claim 1, wherein the compound has the formula: 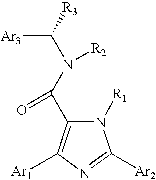
wherein R₃ is C₁-C₆alkyl that is substituted with from 0 to 3 substituents independently chosen from halogen, hydroxy and cyano.

19. A compound or salt according to claim 18, wherein Ar₁ is phenyl or pyridyl, each of which is substituted with from 0 to 3 substituents independently chosen from halogen, C₁-C₄alkyl, haloC₁-C₄alkyl, C₁-C₄alkoxy, C₁-C₄alkylthio and haloC₁-C₄alkoxy.

20. A compound or salt according to claim 18, wherein Ar₂ is phenyl or pyridyl, each of which is unsubstituted or substituted with a halogen.

21. A compound or salt according to claim 18, wherein Ar₃ is phenyl that is unsubstituted or substituted with halogen, C₁-C₄alkyl or C₁-C₄alkoxy.

22. A compound or salt according to claim 18, wherein R₁ is C₁-C₄alkyl and R₂ is hydrogen or C₁-C₄alkyl.

23-25. (canceled)

26. A pharmaceutical composition comprising a compound or salt according to claim 1 in combination with at least one physiologically acceptable carrier or excipient.

27. A packaged pharmaceutical composition comprising the pharmaceutical composition according to claim 16 in a container and instructions for using the composition to treat a patient suffering from a disorder responsive to NK-3 receptor antagonism.

28. A packaged pharmaceutical composition according to claim 27, wherein the patient is suffering from anxiety, depression, psychosis, obesity, chronic pulmonary obstructive disorder, irritable bowel syndrome, colitis, pain or a cognitive disorder.

29. A method for the treatment of a disease or disorder that is responsive to NK-3 receptor modulation, comprising administering to a patient in need of such treatment or prevention a therapeutically effective amount of a compound or salt according to claim 1.

30. A method according to claim 29 wherein the disease or disorder is anxiety, depression, psychosis, obesity, chronic pulmonary obstructive disorder, irritable bowel syndrome, colitis, pain or a cognitive disorder.

31-40. (canceled)
